SEQUENCE LISTING (107 sequences)

SEQ ID NO: 1 - RNA, 22 nt, Artificial Sequence
  Synthetic RNA
ugagguaguagguuguauaguu

SEQ ID NO: 2 - RNA, 22 nt, Artificial Sequence
  Synthetic RNA
ugagguaguagguugugugguu

SEQ ID NO: 3 - RNA, 22 nt, Artificial Sequence
  Synthetic RNA
ugagguaguagguuguaugguu

SEQ ID NO: 4 - RNA, 22 nt, Artificial Sequence
  Synthetic RNA
agagguaguagguugcauaguu

SEQ ID NO: 5 - RNA, 22 nt, Artificial Sequence
  Synthetic RNA
ugagguaggagguuguauaguu

SEQ ID NO: 6 - RNA, 22 nt, Artificial Sequence
  Synthetic RNA
ugagguaguagauuguauaguu

SEQ ID NO: 7 - RNA, 22 nt, Artificial Sequence
  Synthetic RNA
ugagguaguaguuuguacaguu

SEQ ID NO: 8 - RNA, 22 nt, Artificial Sequence
  Synthetic RNA
ugagguaguaguuugugcuguu

SEQ ID NO: 9 - DNA, 88 bp, M. tuberculosis
ctgagccaattcatggaccagaacacccgctgtcggggttgacccacaagcgccgactgtcggcgctggggcccggcggtctgtcacg

SEQ ID NO: 10 - DNA, 13 bp, Artificial Sequence
  Synthetic oligonucleotide
gtggtgtgtgtat

SEQ ID NO: 11 - DNA, 13 bp, Artificial Sequence
  Synthetic oligonucleotide
gtggtctgtgtat

SEQ ID NO: 12 - DNA, 13 bp, Artificial Sequence
  Synthetic oligonucleotide
atacacacaccac

SEQ ID NO: 13 - DNA, 14 bp, Artificial Sequence
  Synthetic oligonucleotide
ggtggtgtgtgtat

SEQ ID NO: 14 - DNA, 14 bp, Artificial Sequence
  Synthetic oligonucleotide
atacacacaccacc

SEQ ID NO: 15 - DNA, 14 bp, Artificial Sequence
  Synthetic oligonucleotide
ggtggtgtatgtgt

SEQ ID NO: 16 - DNA, 14 bp, Artificial Sequence
  Synthetic oligonucleotide
ggtggtgtatgtat

SEQ ID NO: 17 - DNA, 23 bp, Artificial Sequence
  Synthetic oligonucleotide
ccaaggtggtgtgtgtatagtga

SEQ ID NO: 18 - DNA, 23 bp, Artificial Sequence
  Synthetic oligonucleotide
agtgaatacacacaccaccttgg

SEQ ID NO: 19 - DNA, 23 bp, Artificial Sequence
  Synthetic oligonucleotide
tcactatacacacaccaccaacc

SEQ ID NO: 20 - DNA, 23 bp, Artificial Sequence
  Synthetic oligonucleotide
ggttggtggtgtgtgtattcact

SEQ ID NO: 21 - DNA, 23 bp, Artificial Sequence
  Synthetic oligonucleotide
ggttggtggtgtatgtgttcact

SEQ ID NO: 22 - DNA, 23 bp, Artificial Sequence
  Synthetic oligonucleotide
ggttggtggtgtatgtattcact

SEQ ID NO: 23 - DNA, 23 bp, Artificial Sequence
  Synthetic oligonucleotide
ccaaggtggtctgtgtatagtga

SEQ ID NO: 24 - DNA, 23 bp, Artificial Sequence
  Synthetic oligonucleotide
agtgaatacacagaccaccttgg

SEQ ID NO: 25 - DNA, 24 bp, Artificial Sequence
  Synthetic oligonucleotide
ctcactatacacacaccaccaacc

SEQ ID NO: 26 - DNA, 24 bp, Artificial Sequence
  Synthetic oligonucleotide
ggttggtggtgtgtgtattcactc

SEQ ID NO: 27 - DNA, 23 bp, Artificial Sequence
  Synthetic oligonucleotide
agtgaatacacacaccaccaacc

SEQ ID NO: 28 - DNA, 24 bp, Artificial Sequence
  Synthetic oligonucleotide
ctcactatacacacaccaccaacc

SEQ ID NO: 29 - DNA, 24 bp, Artificial Sequence
  Synthetic oligonucleotide
ggttggtggtgtgtgtattcactc

SEQ ID NO: 30 - DNA, 23 bp, Artificial Sequence
  Synthetic oligonucleotide
ccaaggtggtgtgtgtatagtga

SEQ ID NO: 31 - DNA, 23 bp, Artificial Sequence
  Synthetic oligonucleotide
agtgaatacacacaccaccttgg

SEQ ID NO: 32 - DNA, 23 bp, Artificial Sequence
  Synthetic oligonucleotide
ccaaggtggtctgtgtatagtga

SEQ ID NO: 33 - DNA, 23 bp, Artificial Sequence
  Synthetic oligonucleotide
agtgaatacacagaccaccttgg

SEQ ID NO: 34 - DNA, 23 bp, Artificial Sequence
  Synthetic oligonucleotide
ccaaggtggtatgtgtatagtga

SEQ ID NO: 35 - DNA, 23 bp, Artificial Sequence
  Synthetic oligonucleotide
agtgaatacacataccaccttgg

SEQ ID NO: 36 - DNA, 23 bp, Artificial Sequence
  Synthetic oligonucleotide
ccaaggtggtttgtgtatagtga

SEQ ID NO: 37 - DNA, 23 bp, Artificial Sequence
  Synthetic oligonucleotide
agtgaatacacaaaccaccttgg

SEQ ID NO: 38 - DNA, 24 bp, Artificial Sequence
  Synthetic oligonucleotide
ccaaggtggtagtgtgtatagtga

SEQ ID NO: 39 - DNA, 24 bp, Artificial Sequence
  Synthetic oligonucleotide
agtgaatacacactaccaccttgg

SEQ ID NO: 40 - DNA, 24 bp, Artificial Sequence
  Synthetic oligonucleotide
ccaaggtggtggtgtgtatagtga

SEQ ID NO: 41 - DNA, 24 bp, Artificial Sequence
  Synthetic oligonucleotide
agtgaatacacaccaccaccttgg

SEQ ID NO: 42 - DNA, 24 bp, Artificial Sequence
  Synthetic oligonucleotide
ccaaggtggtcgtgtgtatagtga

SEQ ID NO: 43 - DNA, 24 bp, Artificial Sequence
  Synthetic oligonucleotide
agtgaatacacacgaccaccttgg

SEQ ID NO: 44 - DNA, 24 bp, Artificial Sequence
  Synthetic oligonucleotide
ccaaggtggttgtgtgtatagtga

SEQ ID NO: 45 - DNA, 24 bp, Artificial Sequence
  Synthetic oligonucleotide
agtgaatacacacaaccaccttgg

SEQ ID NO: 46 - DNA, 22 bp, Artificial Sequence
  Synthetic oligonucleotide
ccaaggtggttgtgtatagtga

SEQ ID NO: 47 - DNA, 22 bp, Artificial Sequence
  Synthetic oligonucleotide
agtgaatacacaaccaccttgg

SEQ ID NO: 48 - DNA, 23 bp, Artificial Sequence
  Synthetic oligonucleotide
ccaagggggtgtgtgtatagtga

SEQ ID NO: 49 - DNA, 23 bp, Artificial Sequence
  Synthetic oligonucleotide
agtgaatacacacacccccttgg

SEQ ID NO: 50 - DNA, 24 bp, Artificial Sequence
  Synthetic oligonucleotide
ccaaggctggtgtgtgtatagtga

SEQ ID NO: 51 - DNA, 24 bp, Artificial Sequence
  Synthetic oligonucleotide
agtgaatacacacaccagccttgg

SEQ ID NO: 52 - DNA, 22 bp, Artificial Sequence
  Synthetic oligonucleotide
ccaaggggtgtgtgtatagtga

SEQ ID NO: 53 - DNA, 22 bp, Artificial Sequence
  Synthetic oligonucleotide
agtgaatacacacaccccttgg

SEQ ID NO: 54 - DNA, 23 bp, Artificial Sequence
  Synthetic oligonucleotide
ccaaggtggtgtgtgtgtagtga

SEQ ID NO: 55 - DNA, 23 bp, Artificial Sequence
  Synthetic oligonucleotide
agtgaacacacacaccaccttgg

SEQ ID NO: 56 - DNA, 24 bp, Artificial Sequence
  Synthetic oligonucleotide
ccaaggtggtgtgtgtaatagtga

SEQ ID NO: 57 - DNA, 24 bp, Artificial Sequence
  Synthetic oligonucleotide
agtgaattacacacaccaccttgg

SEQ ID NO: 58 - DNA, 22 bp, Artificial Sequence
  Synthetic oligonucleotide
ccaaggtggtgtgtgttagtga

SEQ ID NO: 59 - DNA, 22 bp, Artificial Sequence
  Synthetic oligonucleotide
agtgaaacacacaccaccttgg

SEQ ID NO: 60 - DNA, 35 bp, Artificial Sequence
  Synthetic oligonucleotide
ccaccagaccgcctgggccgagtgcggagatacga

SEQ ID NO: 61 - DNA, 80 bp, Artificial Sequence
  Synthetic oligonucleotide
cgtttgtgcgtaatctcagacagcgggttgttctggtccataaactgagacagctggctggaaccgaagaactctcactc

SEQ ID NO: 62 - DNA, 50 bp, Artificial Sequence
  Synthetic oligonucleotide
gattacgcacaaacgtcgtatctccgcactcggcccaggcggtctggtgg

SEQ ID NO: 63 - DNA, 65 bp, Artificial Sequence
  Synthetic oligonucleotide
ctcactgagttcttcggttccagcctgtctcagtttatggaccagaaccagaacccgctgtctga

SEQ ID NO: 64 - DNA, 35 bp, Artificial Sequence
  Synthetic oligonucleotide
ggctccgtattcgttagtctgtgcgtacacggaca

SEQ ID NO: 65 - DNA, 65 bp, Artificial Sequence
  Synthetic oligonucleotide
gagagttgatcagaccgatgttcggaccttcaggggtttcgattggacatacgcgaccgatggtg

SEQ ID NO: 66 - DNA, 47 bp, Artificial Sequence
  Synthetic oligonucleotide
tgatcaactctctgtccgtgtacgcacagactaacgaatacggagcc

SEQ ID NO: 67 - DNA, 53 bp, Artificial Sequence
  Synthetic oligonucleotide
gtggtacggtcgcgtatgtccaatcgaaacccctgaaggtccgaacatcggtc

SEQ ID NO: 68 - DNA, 63 bp, Artificial Sequence
  Synthetic oligonucleotide
ccactcgttagtctgtgcgtacacggacagagagttgatcagaccgatgttcggaccttcagg

SEQ ID NO: 69 - DNA, 180 bp, Artificial Sequence
  Synthetic oligonucleotide
ggtttcgattggacatacgcgaccgtagtgagtcgggtgtacgtctcgaacttcgaagcctgcacgttcacgggtcagaccgcctgggccgagtgcggagatacgacgtttgtgcgtaatctcagacagcgggttgttctggtccataaactgagacagctggctggaaccgaaatggtg

SEQ ID NO: 70 - DNA, 43 bp, Artificial Sequence
  Synthetic oligonucleotide
tctgatcaactctctgtccgtgtacgcacagactaacgagtgg

SEQ ID NO: 71 - DNA, 200 bp, Artificial Sequence
  Synthetic oligonucleotide
gtggtattcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcgg

SEQ ID NO: 72 - DNA, 21 bp, Artificial Sequence
  Synthetic oligonucleotide
atgatatcgaccacctcggca

SEQ ID NO: 73 - DNA, 28 bp, Artificial Sequence
  Synthetic oligonucleotide
acgtagttgccttcttcgatagcagaca

SEQ ID NO: 74 - DNA, 26 bp, Artificial Sequence
  Synthetic oligonucleotide
agtgagagttcttcggttccagccag

SEQ ID NO: 75 - DNA, 26 bp, Artificial Sequence
  Synthetic oligonucleotide
tcactgagttcttcggttccagccag

SEQ ID NO: 76 - DNA, 19 bp, Artificial Sequence
  Synthetic oligonucleotide
ggtgcagaccgcctgggcc

SEQ ID NO: 77 - DNA, 24 bp, Artificial Sequence
  Synthetic oligonucleotide
accatcggtcgcgtatgtccaatc

SEQ ID NO: 78 - DNA, 24 bp, Artificial Sequence
  Synthetic oligonucleotide
tggtacggtcgcgtatgtccaatc

SEQ ID NO: 79 - DNA, 30 bp, Artificial Sequence
  Synthetic oligonucleotide
ccgaccgtattcgttagtctgtgcgtacac

SEQ ID NO: 80 - DNA, 21 bp, Artificial Sequence
  Synthetic oligonucleotide
caccatttcggttccagccag

SEQ ID NO: 81 - DNA, 21 bp, Artificial Sequence
  Synthetic oligonucleotide
gtggtattcggttccagccag

SEQ ID NO: 82 - DNA, 23 bp, Artificial Sequence
  Synthetic oligonucleotide
gtgtcgttagtctgtgcgtacac

SEQ ID NO: 83 - DNA, 24 bp, Artificial Sequence
  Synthetic oligonucleotide
ccaaggtggtgtgtgtatagtgag

SEQ ID NO: 84 - DNA, 24 bp, Artificial Sequence
  Synthetic oligonucleotide
gagtgaatacacacaccaccttgg

SEQ ID NO: 85 - DNA, 19 bp, Artificial Sequence
  Synthetic oligonucleotide
ggtggtgtgtgtatagtga

SEQ ID NO: 86 - DNA, 19 bp, Artificial Sequence
  Synthetic oligonucleotide
agtgaatacacacaccacc

SEQ ID NO: 87 - DNA, 20 bp, Artificial Sequence
  Synthetic oligonucleotide
ctcactatacacacaccacc

SEQ ID NO: 88 - DNA, 20 bp, Artificial Sequence
  Synthetic oligonucleotide
ggtggtgtgtgtattcactc

SEQ ID NO: 89 - DNA, 19 bp, Artificial Sequence
  Synthetic oligonucleotide
ggtggtctgtgtatagtga

SEQ ID NO: 90 - DNA, 19 bp, Artificial Sequence
  Synthetic oligonucleotide
agtgaatacacagaccacc

SEQ ID NO: 91 - DNA, 20 bp, Artificial Sequence
  Synthetic oligonucleotide
ctcactatacacacaccacc

SEQ ID NO: 92 - DNA, 25 bp, Artificial Sequence
  Synthetic oligonucleotide
gagcgatacacacaccacctcgctc

SEQ ID NO: 93 - DNA, 28 bp, Artificial Sequence
  Synthetic oligonucleotide
gagcgctatacacacaccaccttcgctc

SEQ ID NO: 94 - DNA, 25 bp, Artificial Sequence
  Synthetic oligonucleotide
gagcgacgtttgtgcgtaatcgctc

SEQ ID NO: 95 - DNA, 51 bp, Artificial Sequence
  Synthetic oligonucleotide
gaacaacccgctgtctgagattacgcacaaacgtcgtatctccgcactcgg

SEQ ID NO: 96 - DNA, 114 bp, Artificial Sequence
  Synthetic DNA
agtgagagttcttcggttccagccagctgtctcagtttatggaccagaacaacccgctgtctgagattacgcacaaacgtcgtatctccgcactcggcccaggcggtctgcacc

SEQ ID NO: 97 - DNA, 114 bp, Artificial Sequence
  Synthetic DNA
ggtgcagaccgcctgggccgagtgcggagatacgacgtttgtgcgtaatctcagacagcgggttgttctggtccataaactgagacagctggctggaaccgaagaactcagtga

SEQ ID NO: 98 - DNA, 115 bp, Artificial Sequence
  Synthetic DNA
ccaccacaccgcctgggccgagtgcggagatacgacgtttgtgcgtaatctcagacagcgggttgttctggtccataaactgagacagctggctggaaccgaagaactctcactc

SEQ ID NO: 99 - DNA, 115 bp, Artificial Sequence
  Synthetic DNA
ctcactgagttcttcggttccagccagctctctcagtttatggaccagaacaacccgcacagagagattacgcacaaacgtcgtatctccgcactcggcccaggcggtgtggtgg

SEQ ID NO: 100 - DNA, 99 bp, Artificial Sequence
  Synthetic DNA
accatcggtcgcgtatgtccaatcgaaacccctgaaggtccgaacatcggtctgatcaactctctgtccgtgtacgcacagactaacgaatacggtcgg

SEQ ID NO: 101 - DNA, 99 bp, Artificial Sequence
  Synthetic DNA
ccgaccgtattcgttagtctgtgcgtacacggacagagagttgatcagaccgatgttcggaccttcaggggtttcgattggacatacgcgaccgtacca

SEQ ID NO: 102 - DNA, 100 bp, Artificial Sequence
  Synthetic DNA
ggctccgtattcgttagtctgtgcgtacacggacagagagttgatcagaccgatgttcggaccttcaggggtttcgattggacatacgcgaccgatggtg

SEQ ID NO: 103 - DNA, 100 bp, Artificial Sequence
  Synthetic DNA
gtggtacggtcgcgtatgtccaatcgaaacccctgaaggtccgaacatcggtctgatcaactctctgtccgtgtacgcacagactaacgaatacggagcc

SEQ ID NO: 104 - DNA, 242 bp, Artificial Sequence
  Synthetic DNA
accatttcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgacacc

SEQ ID NO: 105 - DNA, 242 bp, Artificial Sequence
  Synthetic DNA
ggtgtcgttagtctgtgcgtacacggacagagagttgatcagaccgatgttcggaccttcaggggtttcgattggacatacgcgaccgtagtgagtcgggtgtacgtctcgaacttcgaagcctgcacgttcacgggtcagaccgcctgggccgagtgcggagatacgacgtttgtgcgtaatctcagacagcgggttgttctggtccataaactgagacagctggctggaaccgaatacca

SEQ ID NO: 106 - DNA, 243 bp, Artificial Sequence
  Synthetic DNA
ccactcgttagtctgtgcgtacacggacagagagttgatcagaccgatgttcggaccttcaggggtttcgattggacatacgcgaccgtagtgagtcgggtgtacgtctcgaacttcgaagcctgcacgttcacgggtcagaccgcctgggccgagtgcggagatacgacgtttgtgcgtaatctcagacagcgggttgttctggtccataaactgagacagctggctggaaccgaaatggtg

SEQ ID NO: 107 - DNA, 243 bp, Artificial Sequence
  Synthetic DNA
gtggtattcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgagtgg

(19) United States
(12) Patent Application Publication
(10) Pub. No.: US 2014/0302486 A1
(43) Pub. Date: Oct. 9, 2014

(54) SYSTEMS AND METHODS FOR DETECTING BIOMARKERS OF INTEREST

(71) Applicants: President and Fellows of Harvard College, (unknown); University of Washington Through Its Center For Commercialization, Seattle, WA (US)

(72) Inventors: Georg Seelig, Seattle, WA (US); Xi Chen, Seattle, WA (US); David Yu Zhang, Houston, TX (US)

(21) Appl. No.: 14/139,464

(22) Filed: Dec. 23, 2013

Related U.S. Application Data
(63) Continuation-in-part of application No. 13/603,298, filed on Sep. 4, 2012.
(60) Provisional application No. 61/740,869, filed on Dec. 21, 2012.
(60) Provisional application No. 61/530,739, filed on Sep. 2, 2011.

(51) Int. Cl. C12Q 1/68

(52) U.S. Cl. CPC C12Q 1/6818; C12Q 1/6876; C12Q 1/6827
USPC 435/5; 536/24.3; 436/501; 435/6.11

(57) ABSTRACT

A detection probe for detecting single base mutations or alterations in a double stranded target nucleic acid molecule is provided. In some aspects, the detection probe may include a double-stranded probe nucleic acid molecule having a first end and a second end; at least one probe initiation toehold at the first end; at least one substance capable of emitting a detectable signal at the second end; and optionally, at least one dissociation toehold at the second end. The detection probe is designed to hybridize with a double stranded target nucleic acid in a reaction which proceeds at approximate thermodynamic equilibrium (ΔG≈0) when no single base mutations or alterations are present in the double stranded target nucleic acid molecule. The probe may be used in detection systems and methods to identifying the presence or absence of one or more single base mutations or alterations in a double-stranded nucleic acid target molecule.

### PRIORITY CLAIM

This application claims priority to U.S. Provisional Patent Application Ser. No. 61/740,869, filed Dec. 21, 2012, and is a continuation in part of U.S. patent application Ser. No. 13/603,298, filed Sep. 4, 2012, which claims priority to U.S. Provisional Patent Application Ser. No. 61/530,739, filed Sep. 2, 2011, the subject matter of all of which is hereby incorporated by reference as if fully set forth herein.

### STATEMENT OF GOVERNMENT INTEREST

The present invention was made with government support under Grant No. CBET-1041548, awarded by the National Science Foundation (NSF). The Government has certain rights in the invention.

### BACKGROUND

Nucleic acids are the genetic signature molecules for all life; sequences and expression levels of biological nucleic acids provide information about the state of an individual cell or a whole organism. Often, small differences (even single base changes) between otherwise identical nucleic acid sequences can have important biological and biomedical implications. Single base mutations, such as insertions, deletions and single nucleotide polymorphisms (SNPs), form the genetic basis for a variety of human diseases (Gunderson et al. 2005; Kim & Misra 2007) or can confer drug resistance to pathogenic bacteria or viruses (Arnold et al. 2005; Bang et al. 2011). Consequently, the fast, simple and accurate detection, analysis and quantitation of nucleic acid sequences with single base resolution are important research goals with vast potential for biomedical applications.

Virtually all nucleic acid detection technologies utilize the specificity of Watson-Crick base pairing; single-stranded probe (Schena et al. 1995) or primer (Saiki et al. 1988; Shendure et al. 2005) molecules capture intended DNA or RNA target molecules of complementary sequence. However, cross-hybridization between closely related probe-target pairs can occur. Such imperfect probe-target binding may be trapped kinetically (i.e., dissociate slowly) and practically prevent the intended hybridization reactions. To achieve single-base specificity, many diagnostic assays exploit the sensitivity of enzymes (Landegren et al. 1988; Tong et al. 2001; Botstein et al. 1980; Hall et al. 2000) to the presence of mismatch bubbles within their DNA substrates. DNA-templated, non-enzymatic ligation reactions can also be highly sensitive to point mutations in the template (Xu et al. 201; Grossman & Seitz 2009).

Alternatively, the complementary probe molecules themselves can be engineered to possess greater specificity to the intended target molecule. For example, incorporation of chemically modified bases (Singh et al. 1998; Egholm et al. 1992) can increase the affinity of a probe to a correct target. Consequently, disruption of correct base pairing results in a higher energetic penalty and better discrimination (Simeonov & Nikiforov 2002; Komiyama et al. 203). Hairpins and other structural elements can be introduced into the probe molecule to make binding to both the correct and SNP target less energetically favorable, such that small energetic differences can result in strong differences in the hybridization yield (Tyagi & Kramer 1996; Zhang et al. 2009; Guo et al. 1997; Zhang & Winfree 2009; Xiao et al. 2009; Tyagi 2009; Manganelli et al. 2001). However, reaction conditions often need to be finely tuned for optimal performance, and it is difficult to assay long nucleic acid molecules for single base changes.

Pathogen-derived proteins and nucleic acids have important roles as diagnostic markers for infectious diseases. For example, the detection of just a single molecular marker, such as a DNA sequence associated with the M. tuberculosis complex, can sometimes be indicative of a disease state. However, a reliable diagnosis and treatment decision often requires interpreting a combination of markers via complex algorithms. For example, in the case of DNA testing for tuberculosis (TB), the treatment decision requires interpretation of markers associated with the M. tuberculosis complex, TB antibiotic resistance and even HIV-coinfection (McNerney et al., 2011).

DNA-based circuits and reaction networks may be designed that can be used for the analysis of complex molecular mixtures. Synthetic molecular circuits that are capable of information processing and computation have been built using a range of approaches. Examples include synthetic gene regulatory and signaling networks (Isaacs et al., 2006; Yeh et al., 2007; Win et al., 2008), computational networks using in vitro transcription (Kim et al., 2006; Simpson et al., 2009), digital logic circuits based on small molecules (de Silva et al., 2007) or peptides (Ashkenasy et al., 2004), and the nonlinear chemical reaction networks underlying the Belousov Zhabotinskii reaction and related phenomena (Epstein et al., 1998). In these circuits, information is stored in the concentrations, spatial localizations, and/or chemical properties of molecules; chemical reactions between molecules implement molecular information processing. Most of these systems lack the flexibility and modularity that would make them useful for biosensing applications. For proteins and small molecules in particular, the de novo design of individual functional molecular sensors and logic gates is difficult and integration of multiple elements into circuits is even more challenging.

Because of the predictability of Watson-Crick base pairing, nucleic acid-based systems avoid some of these constraints and can be used to implement modular and scalable molecular computation. Initial demonstrations of nucleic acid logic circuits took advantage of enzyme or deoxyribozyme catalysis (Lu et al., 2006; Willner et al., 2008). Also, a DNA and enzyme-based molecular automaton was developed that could perform a computation where the outcome (the release of an antisense drug mimic) was dependent on the absence or presence of specific inputs (ssDNA with sequence analogous to diagnostically relevant mRNA) (Benenson et. al., 2001; Benenson et al., 2004). Stojanovic and collaborators developed deoxyribozyme based logic gates (Stojanovic et al, 2002) and used these gates to form a variety of logic circuits (Stojanovic et al., 2003; Lederman et al, 2006; Yashin et al., 2007). Penchovsky and Breaker (Penchovsky et al., 2005) developed allosteric ribozymes that could implement cascaded logic using DNA inputs and RNA outputs. More recent work (Takahashi et al, 2006; Frezza et al., 2007; Cardelli et al., 2008; Qian et al., 2011; Seelig et al., 2006; Soloveichik et al., 2010), has relied on hybridization and strand displacement as a mechanism for implementing molecular logic.

Although the field of DNA-based circuits and reaction networks has several promising approaches, such approaches are limited by their ability to discriminate between closely related molecules, especially when the sequence of the related molecules differs by a small number nucleotides. Thus, it would be desirable to design a system capable of robustly distinguishing molecules having related or similar sequences.

### SUMMARY

A detection probe for detecting single base mutations or alterations in a double stranded target nucleic acid molecule is provided in accordance with the embodiments described herein. In some aspects, the detection probe may include a double-stranded probe nucleic acid molecule having a first end and a second end; at least one probe initiation toehold at the first end; at least one substance capable of emitting a detectable signal at the second end; and optionally, at least one dissociation toehold at the second end. The detection probe is designed to hybridize with a double stranded target nucleic acid in a reaction which proceeds at approximate thermodynamic equilibrium (ΔG≈0) when no single base mutations or alterations are present in the double stranded target nucleic acid molecule.

In some embodiments, a single base mutation or alteration detection system is provided herein. The detection system may include a detection probe which comprises (i) a double stranded probe nucleic acid molecule having a first end and a second end; (ii) at least one probe initiation toehold at the first end; and (iii) at least one substance capable of emitting a detectable signal attached to its second end. The detection probe is designed to hybridize with a double stranded target nucleic acid in a reaction which proceeds at approximate thermodynamic equilibrium (ΔG≈0) when no single base mutations or alterations are present in the double stranded target nucleic acid molecule. Further, the detection probe hybridizes with the double stranded target nucleic acid in a reaction which proceeds at ΔG<0 when one or more single base mutations or alterations are present in the double stranded target nucleic acid molecule.

The detection system may also include a double stranded target nucleic acid molecule comprising (i) a first end and a second end; and (ii) at least one target initiation toehold at its first end that is complimentary to the probe initiation toehold. In addition, the detection system may include at least one dissociation toehold at the second end of the detection probe, the second end of the target, or both.

In some embodiments, a method of identifying the presence or absence of one or more single base mutations or alterations in a double-stranded nucleic acid target molecule is provided. Such a method may include steps of reacting a detection probe with a double-stranded nucleic acid target molecule to produce a reaction product which produces a detectable signal; measuring a level of the detectable signal of the reaction product; and determining a target hybridization yield via the level of detectable signal. According to some aspects, a target hybridization yield of greater than approximately 10% indicates the absence of one or more single base mutations or alterations in the double-stranded nucleic acid target molecule. According to some aspects, a target hybridization yield of less than approximately 10% indicates the presence of one or more single base mutations or alterations in the double-stranded nucleic acid target molecule.

### BRIEF DESCRIPTION OF THE DRAWINGS

FIG. 1 illustrates examples of double-stranded toehold exchange probes. (A) shows that hybridization is the binding process of complementary single-stranded nucleic acids. Formation of base pairs provides energy gain during hybridization. When there is a mismatch, the system will gain less energy, but the reaction still goes forward. (B) shows an example of strand displacement, which is the process of using one strand to displace another one. The single-stranded input binds to a double-stranded complex using partially single-stranded regions which are called toeholds (green, 1a, 1b). Because the black regions are identical for both top strands, a three way branch migration will start. The top strand, which originally binds the bottom strand, will fall off. (C) by adding a toehold on the other end (blue, 2a, 2b), the strand that fell off in (B) may re-bind the bottom strand, making the reaction reversible. This binding exchange is called toehold exchange. During this process, there is almost no enthalpy change and very few entropy change independent of environment. This method has been used to characterize nucleic acid dynamics, build catalysts, and make specific detection probes. (D) shows a four way strand displacement, which happens between four single-stranded nucleic acid and two sets of binding. The junction between four strands is called a holliday junction, and it represents an intermediate stage in genetic recombination.

FIG. 2 illustrates how strand displacement can be used to detect single-stranded nucleic acids, according to some embodiments. (A) A strand displacement reaction where an input strand (1:2:3) displaces an output strand (2:3:4) from a gate/sensor complex. DNA is represented as directional lines, with the arrow denoting the 3′ end. Domains are labeled by numbers, with _ denoting Watson-Crick complementarity. Multiple elementary steps are indicated: (1) binding of toeholds 1 and 1*; (2) a random walk branch migration process where domain 2 of strand 2:3:4 is partially displaced by domain 2 of strand 1:2:3; (3) the separation of domains 3 and 3* and release of an output strand. (B) The output strand of one strand displacement reaction can serve as an input to a downstream reaction. In this case, a reporter complex is used to detect the output strand released in the upstream reaction. The two strands in the reporter complex are chemically labeled with a fluorophore (magenta star) and a quencher (dark blue dot), respectively. Separation of the fluorophore and quencher lead to an increase in fluorescence. (C) Example kinetics traces for the two-step reaction sketched in (A) and (B). Gate complex is at 10 nM, readout at 13 nM, input concentration is varied from 0 nM to 10 nM. Reactions are run at 25° C. in TAE buffer with 12.5 mM Mg++. Domain 1 is 5 nucleotides (nt), domains 2 and 4 are 15 nt and domain 3 is 6 nt. The data can be fit well with a simple model assuming two sequential bimolecular reactions. (D) Strand displacement kinetics strongly depends on the free energy released when domains 1 and 1* hybridize. Reaction rates increase over several orders of magnitude as the toehold length is increased from 0-6 nt.

FIG. 3 illustrates a catalytic system that includes a fuel molecule. The reaction starts with the toehold binding of the Gate and the Input (catalyst), followed by branch migration. The Output strand will be released from the Gate through branch migration, and the hidden toehold is exposed. A single-stranded Fuel molecule may bind on the exposed toehold and release the Input molecule. The Input can then be reused to trigger release of more Output strands to enhance the signal. The Output strands may react with a reporter to produce a signal.

FIG. 4 illustrates that a seesaw gate may be used in a system for catalytic signal amplification, in accordance with some embodiments, which provides the basis for the biosensing systems provided herein. (A) The amplifier includes one double-stranded complex (“gate”) and an auxiliary strand “fuel.” The catalyst strand initiates the reaction by binding the gate through toehold mediated strand displacement. In the Examples provided herein, the role of the catalyst is played by a single-stranded biomarker of interest. (B) The reaction mechanism for the seesaw gate is initiated by the catalyst. The catalyst (1:2) binds the double stranded gate, resulting in release of the output (2:3:4). Next, the fuel strand (2:3) binds and releases the catalyst. See Qian et al., 2011 for more details regarding the seesaw gate system. (C) In this catalytic signal amplification experiment, the gate and fuel are at 10 nM and the catalyst concentration is varied. A readout gate is used to detect release of the output strand. The concentration of the readout is 13 nM.

FIG. 5 shows a logic circuit using a combination of microRNA and related precursor inputs and corresponding gates. (A) Signal propagation through an in vitro chemical circuit combining AND, OR, sequence translation, input amplification, and signal restoration. The five-layer circuit includes a total of 11 gates and accepts six inputs. Corresponding DNA structures are shown with the circuit diagram (inset). (B) Fluorescence traces of circuit operation without and with the signal restoration module (threshold plus amplifier). The traces for input conditions corresponding to a logical TRUE output (ON) are clearly distinguishable from the logical FALSE output (OFF). Cases tested include when all inputs are present, all cases in which exactly one input is missing, and combinations of inputs that turn off an OR clause.

FIG. 6 is a schematic diagram showing a variety of diagnostic tests available, including lateral flow tests, instrumented PCR, line probe assays, and DNA/RNA microarrays. The diagnostics tests are arranged in accordance with their strengths and suitable use. Tests that are show farthest to the right are most suitable for a large numbers of markers that can improve diagnosis and provide comparative results, but require complex interpretation algorithms. Those shown toward the left are most suitable for use with single biomarkers and provide for easy to use, rapid results.

FIG. 7 shows a series of multi-step processes in paper networks, according to some embodiments. (A) Sugar solutions dried on each leg create fluidic time delays that can be controlled by the sugar concentration. Each fluid source has a limited volume so it shuts off at a programmed time. The result is delivery of multiple fluids to a detection zone in a timed sequence. (B) A folding card design is used to contact fluid source pads to the network for a single step initiation of the fluid sequence. (C) Fluid from each leg is delivered in a timed sequence on a time scale appropriate for rapid point of care assays (30 minutes). Reagents can be stored in dry form on the pads, such that the user only adds a sample and buffer or water. Alternative designs have been used to perform automated assay sequences for wash steps and chemical signal amplification.

FIG. 8 shows a mechanism of detecting mismatch discrimination according to some embodiments. (A) A combination of a seesaw amplifier and a “sink” complex can be used to distinguish catalyst strand that differs by only a single base. (B) A seesaw amplifier without a sink can be used for kinetic discrimination of the inputs. However, end-point discrimination is not possible. Amplifier components are 10 nM, readouts are 13 nM and all catalysts are 5 nM. Experiments are performed in TAE with 12.5 mM Mg++. (C) The input can be inhibited by addition of a sink. When the input binds the sink and completes branch migration, the input will not be released again. (D) Gates and sinks have different binding affinities for binding correct and mismatched targets. The correct input (catalyst) binds to the amplifier gate faster than it binds to the inhibitory sink. Although the input may be degraded slowly by the sink, a fast catalytic reaction of the input and gate ensures completion of amplification. The mutated input (catalyst with mismatched mutation) will be inhibited rapidly and thus almost no signal will be triggered. (E) Combining the seesaw amplifier with a sink enables endpoint discrimination. Reaction conditions are similar to those in (b). The sink concentration is 10 nM. (F) Kinetics with and without the mismatch can be predicted using binding energy. Reactions involving mis-binding are significantly slower. For idea rapid endpoint discrimination, the correct catalyst toehold binding should have energy around 10 kcal/mol.

FIG. 9 shows a system for detection of DNA analogs of the let-7 family according to some embodiments. (A) The let-7 family is a set of miRNAs that have very similar sequences (SEQ ID NOs:1-9). (B) Experimental results from the let-7 detection system indicate that the catalytic system for let-A can be triggered by several other let-7 mismatched inputs. (C) The same catalytic system as shown in (B) is prepared using various sinks to sequester the mismatched let-7 family members. When sinks are added to the reaction, clear endpoint discrimination is seen for correct and mismatched inputs. With the sink, mismatched catalysts are degraded, producing little signal which is similar to that seen with a system leak. (D) Two catalytic systems, let-A and let-C, were run in parallel using different output channels, the results of which are shown in (E).

FIG. 10 illustrates double-stranded toehold exchange probes. (A) shows a double toehold exchange probe P (or PtPb), which includes a fork shaped toehold. Such toeholds help initiate four-way branch migration. In this case, 5 new base pairs (a1, a2 and b1, b2) are formed through the course of the reaction, and 4 base pairs (c1, c2 and d1. D2 single-stranded regions) and a fluorophore-quencher interaction are broken. Toeholds before and after the reaction have similar lengths so that the standard free energy of the forward reaction is about 0 kcal/mol. Thus, when the correct target reacts with the probe, roughly equal amounts of reactant and products are seen at equilibrium. (B) shows a double-stranded toehold exchange reaction with a spurious target S (or StSb). This forward four-way branch migration at the mutated position is very slow and not favorable due to the energy penalty caused by mismatch binding. The intermediate step shown possesses a much higher backward reaction rate constant. Consequently, there should be much fewer products at equilibrium.

FIG. 11 shows the results of a fluorescence assay. (A) shows sequences of correct and single-base-changed targets, and a corresponding probe. The probe is functionalized with the ROX fluorophore and the Iowa Black Red Quencher. (B) shows the kinetic traces of the reaction between the probe and the correct target (top) or spurious insertion target (bottom) with various concentrations of target. In all traces, [PtPb]=10 nM. (C) shows a log-log plot of hybridization yield X as a function of the concentration of the target. Dots represent experimental values of X, and lines represent the analytic relationship between X and [Target], using a least-squares fit for the y-intercept (X at equal stoichiometry). Discrimination factor (Q) is defined as Q=(X_{Correct})/(X_{mutated}), and concentration tolerance (R) is defined as R=([TtTb]_{X=0.5})/([StSb]_{X=0.5}), the ratio of concentrations needed to achieve 50% yield at equilibrium.

FIG. 12 shows robustness over mutation position identities, as well as temperature and salinity. (A) shows tested mutation position and identities. (B) shows a kinetics trace for all correct targets (Correct) and mutated targets (All SNPs). The mutated targets give little to no signal. (C) shows the discrimination factor (Q) as measured for the targets shown. The discrimination factor is defined as Q=(X_{Correct})/(X_{mutated}), and has a median of 43. (D) shows that mutated target m8G-C was tested under different temperature and salinity.

FIG. 13 shows the detection of a mutation in the RpoB gene from M. tuberculosis (TB). (A) shows the subsequence of TB (SEQ ID NO:9). Two systems of different length and sources were tested. The short 50 nt target was made synthetically, and the 100 nt target was made from a plasmid containing the subsequence of TB using PCR. (B) shows testing of one mutation at position 526 with a s50 nt TB subsequence under various concentrations as shown. (C) shows single strands with a desired toehold end could be generated from unbalanced PCR, wherein one primer has 100 times lower concentration than ordinary PCR. Annealing the two single strands, the double strand with toeholds may be generated. (D) shows the results of testing the purified annealing product.

FIG. 14 is a schematic representation of the double-stranded toehold exchange mechanism according to one embodiment. (a) shows that the reaction starts with the hybridization of the initiation toeholds (orange: a1, a2; and purple: b1, b2) to form a four-stranded complex C₀. Next, the four-stranded complex undergoes a series of single-base reconfiguration events, known as branch migration (Thompson et al. 1976). The various states of branch migration (Cᵢ) are roughly isoenergetic, and thus each branch-migration step is reversible and unbiased. When the branch migration reaches state Cₙ, at which the four-stranded complex is held together only by the dissociation toeholds (blue: c1, c2 and green: d1, d2), these dissociation toeholds can dissociate spontaneously to release the two product molecules. (b) shows a highly specific, conditionally fluorescent molecular probe based on four-stranded toehold exchange. The probe is functionalized at the balancing toeholds with a fluorophore and a quencher on separate strands; at the end of the reaction, the fluorophore is delocalized from the quencher and fluorescence increases. The lengths and sequences of the toeholds are designed so that the ΔG°_{intended} of the reaction between the probe and the intended target is roughly 0. The reaction between the probe and the SNP target results in two mismatch bubbles, and the reaction ΔG°_{SNP} will be about 8 kcal mol⁻¹. (c) shows the plot of the analytic hybridization yield at equilibrium of the A+B⇄D+E reaction against the reaction ΔG° (assuming identical initial concentrations of A and B). Designing ΔG°_{intended}≈0 ensures a balance of high specificity and high yield.

FIG. 15 shows fluorescence normalization and discrimination factor Q determination according to one embodiment. Shown to the right are sample fluorescence traces for the probe alone (orange), the probe with correct target (black), and the probe with the “m8CG” SNP target (blue). At time t=0, the reaction is started by addition of target, if any. At time t=6.4 hr, the reaction is terminated by addition of a large excess of a direct trigger that reacts with the probe (left panel); this fluorescence value after equilibration of this latter reaction corresponds to 100% reaction yield. The hybridization yields of the correct and SNP targets are calculated as:

$$x = \frac{F - {F}_{b}}{{F}_{max} - {F}_{b}} ,$$

where F denotes the fluorescence value of the reaction after 6.4 hours of reaction, F_{b} denotes the time-varying background fluorescence observed from addition of pure buffer, and Fₘₐₓ denotes the equilibrium after post-trigger.

FIG. 16 shows the discrimination of SNPs by a dsDNA probe according to one embodiment. (a) shows the sequence of the intended target and the positions/identities of base-pair changes that lead to the 14 SNP targets. Circled 1, 2 and 3 denote the positions of the mismatch. Mismatches, insertions and deletions are shown as indicated (in blue, red and green, respectively). ROX, carboxy-X-rhodamine fluorophore; RQ, Iowa Black Red Quencher. (b) shows the hybridization yield as inferred from fluorescence kinetics (see FIG. 15 and Methods). The probe is present in solution initially, and the intended or SNP target is introduced at t≈0. Experiments were run at 25° C. in 1 M Na⁺. The trace for the intended target is shown in black, and traces for SNP targets are shown in the colors described above (see FIG. 37 for a zoom-in of SNP reactions). (c) shows that the reaction equilibration appears to be complete after four hours; to ensure equilibration, however, the reactions were allowed to proceed until t=25 h. The hybridization yields at t=25 h are taken to be the equilibrium values, and discrimination factors Q=x_{intended}/X_{SNP} are calculated for each SNP target. Observed Q values range between 17 and 99 (median=43). Error bars show standard deviations calculated from three repetitions of each experiment.

FIG. 17 shows the concentration of SNP target needed to generate the same x as a stoichiometric (relative to probe) amount of intended target according to one embodiment. (a) shows sequences of intended and SNP targets used for the experiments shown in this Figure. (b) shows the hybridization yields of various concentrations of intended and ‘i8TA’ SNP targets. In all traces, the initial probe concentration [B]₀=10 nM. (c) shows the hybridization yields plotted against the stoichiometric ratio of the target. As with previous experiments, hybridization yield was inferred from the fluorescence value at t=25 h. Experimentally determined values are shown as dots and the star, and solid lines show the analytic model prediction based on best-fit ΔG° values (see Example 3). All experiments other than the star data point were performed with 10 nM probe; the star data point reaction was performed with 2 nM probe to conserve reagents. R values are calculated based on best-fit models at 50% hybridization yield, and range between 260 and 12,000. Analysis shows and experiments verify that R≈Q2, with Q=X_{intended}/x_{SNP} being the discrimination factor.

FIG. 18 shows the characterization of the background, temperature, salinity and time robustness of a probe according to one embodiment. (a-c) shows that the probe operates robustly to discriminate SNPs in the presence of high concentrations of 50 nucleotide polynucleotide strands (a) in different salinity buffers (b) and at different temperatures (c) (see also FIGS. 39 and 39). (d) shows that the discrimination factor Q approaches its final value after about ten minutes of reaction, and maintains a high discrimination indefinitely. The initial rise and bumpiness in Q can be attributed to fluorescence-signal instability directly after the addition of target to solution (See FIG. 37).

FIG. 19 shows the detection of SNPs in E. Coli-derived samples according to one embodiment. (a) shows that Rifampicin resistance is typically conferred by mutations in one of two regions in the rpoB gene, nucleotides 1,531-1,599 and 1,684-1,728, which correspond to amino acid residues 511-533 and 562-576, respectively. Here, three distinct probes were generated, one to test one region, one to test the other, and one to test both simultaneously (see FIG. 40 for sequences of probes and targets). (b) shows that DNA from ten rifampicin-resistant colonies was extracted and individually amplified by colony PCR. Subsequently, unbalanced PCR using an excess of one primer with an overhang was used to generate the initiation toeholds. These DNA samples were allowed to react with the fluorescent probes described herein, which were constructed by annealing four separate oligonucleotides, and possessed non-overlapping nicks that do not interfere with probe function. (c) The left side of each column shows the approximate position of the mutations, as determined by sequencing. The right side of each column shows the fluorescence response of the rpoB subsequences to the two fluorescent probes. A mutation in region 1 or 2 would result in no increase in the fluorescence for Probe 1 or Probe 2, respectively, as shown in the corresponding traces, 1 and 2). The experimental results agree with the sequencing results in all experiments. The fluorescence data shown in the experimental panels on the left represent the behavior over three hours of the reaction; the right panels show ten hours of reaction (see FIGS. 30-34 for zoomed-in view of data).

FIG. 20 is a representative Electrospraylonization (ESI) mass spectrometry spectrum according to one embodiment. The oligonucleotide analyzed here is the fluorophore-labeled strand of the probe.

FIG. 21 is a table showing an ESI summary for oligonucleotides used in FIG. 16, FIG. 17 and FIG. 18.

FIG. 22 is a table showing an ESI summary for oligonucleotides used in FIG. 19.

FIG. 23 is a table showing PCR primers for E. Coli-derived target. Shown in bold are initiation and dissociation toehold domains.

FIG. 24 shows that a probe effectively discriminates correct target from SNP targets regardless of probe concentration, as predicted by theory according to one embodiment. Consistent with analysis and FIG. 17, increased concentrations of SNP targets have a sublinear effect on the hybridization yield.

FIG. 25 illustrates the discrimination of 100 μM intended target from m8CGSNP variant according to one embodiment.

FIG. 26 shows alternate designs for four-way toehold exchange, in which only a subset of the 4 toe holds are used. according to one embodiment The design shown in panel (C) was experimentally tested, which uses exactly 1 initiation toehold and 1 dissociation toehold, and may be easier to prepare than probes with 2 initiation and 2 dissociation toeholds. This probe effectively discriminates intended target from SNP target, although with significantly slower kinetics than the design presented in the main paper.

FIG. 27 shows that probes which exhibit reduced specificity with toehold sequences cause ΔG° to be significantly negative according to one embodiment. Here, the probes with 6 nt initiation toeholds and 4 nt dissociation toeholds show more reaction with 200 nM “m8CG” SNP target than the default probes with 5 nt initiation toeholds.

FIG. 28 shows that double-stranded probes without dissociation toeholds react with both correct and SNP targets with thermodynamic favorability (ΔG°<0) according to one embodiment. Experimental results across a variety of salinities demonstrate that that SNP target react significantly with the probe, leading to a false positive signal.

FIG. 29 is a comparison of the (a) toehold exchange probe with the (b) probe lacking balancing toeholds according to one embodiment. The observed discrimination factor Q is plotted versus time. The values of Q quickly approached their equilibrium values, and in all cases were higher than 10 after 20 minutes. In contrast, the probe lacking balancing toeholds can be used to kinetically discriminate SNPs in the early timepoints of the reaction, but observed discrimination factors Q decrease as the reactions approach equilibrium. Note that even the highest Q observed for the probes lacking balancing toeholds is lower than corresponding Q values for the toehold exchange probes.

FIG. 30 shows the performance of two different molecular beacons on discriminating SNPs according to one embodiment. Compare with FIG. 16b for performance of double-stranded probe. At elevated temperatures (50° C.), molecular beacons show improved specificity, but at the cost of hybridization yield. Molecular beacons targeting a 21 nt sequence were also tested, but these did not show significant difference between correct and SNP targets (data not shown).

FIG. 31 shows the performance of a molecular beacon targeting 15 nt subsequence of the E. Coli rpoB gene, nucleotides 1570-1584 according to one embodiment. Compare with FIG. 19 for performance of double-stranded probe on subsequences ranging from 45 nt to 198 nt. Molecular beacon SNP discrimination performance is significantly worse than in the case of the synthetic targets (FIG. 36), because of secondary structure inherent in the target sequence. In contrast, double-stranded probes are not affected by secondary structure.

FIG. 32 shows the detection of Rif-resistance in E. Coli using double-stranded probes according to one embodiment; wildtype and resistant colonies #1 and #2. (Summarized in FIG. 19).

FIG. 33 shows the detection of Rif-resistance in E. Coli using double-stranded probes according to one embodiment; wildtype and resistant colonies #3 and #4

FIG. 34 shows the detection of Rif-resistance in E. Coli using double-stranded probes according to one embodiment; wildtype and resistant colonies #5 and #6.

FIG. 35 shows the detection of Rif-resistance in E. Coli using double-stranded probes according to one embodiment; wildtype and resistant colonies #7 and #8.

FIG. 36 is a schematic of the targets and probes used in FIG. 19 according to one embodiment. Probes are composed of four strands, rather than 2. The non-overlapping nicks in the probe are not expected to affect performance of the probes, except when the SNP is directly adjacent to the nick. Colored bases show subsequences of the rpoB that are probed; black letters show primers binding sites for PCR amplification. Shaded background (1) highlights complementary non-overlapping nick region.

FIG. 37: shows an expanded view of reaction between probe and SNP targets from FIG. 16, plotted as non-normalized fluorescence according to one embodiment. The reaction of the probe with the correct target is shown in black and rises very quickly with respect to SNP traces. Note that the arbitrary fluorescence units of different Figures differ from each other. The baseline trace shows the average of 4 separate reactions of probe with no target; buffer was added at t=0 to preserve similarity of reaction conditions and correct for dilution effects. Fluorescence value for the baseline and SNP traces showed an initial decline of fluorescence at t<1 hr; this decline is repeatable and likely due to the settling of air bubbles introduced from mixing by repeated pipetting.

FIG. 38 shows that poly-N single-stranded DNA has no significant effect on the thermodynamic or kinetic behavior of the probe reaction, even at high concentration excesses according to one embodiment. This result contrasts that of probe based on three-stranded toehold exchange for discriminating single-stranded DNA and RNA.

FIG. 39 shows the temperature and salinity effects on probe performance according to one embodiment. (a) and (e) show the hybridization yield over the first three hours of reaction for varying salinities and temperatures, respectively. (b) and (f) summarizes the fluorescence after 20 hours of reaction. (c) and (g) summarizes the best-fit rate constants of the forward reaction (A+B⇄D+E) to the kinetic traces shown in panels (a) and (e). (d) and (h) summarizes the discrimination factors Q observed at t=20 hr.

FIG. 40 shows the detection of Rif-resistance in E. Coli using double-stranded probes according to one embodiment; wildtype and resistant colonies #9 and #10.

FIG. 41 shows various methods for diagnosing TB antibiotic resistance according to some embodiments. (A) An example of TB targets and interpretation algorithm (partial set) for identification of resistance (RMP: rifampin, INH: isoniazid). Adapted from the Genotype MTBDR+ line probe assay (Niemz et al., 2011). (B) A logic circuit diagram for a diagnostic circuit for detection of different markers associated with TB antibiotic resistance. The circuit combines an embedded analysis with a simplified readout.

FIG. 42 shows a schematic of a two-stage strip for sequencing DNA circuit reactions and visually detecting output DNA according to some embodiments. Labeled outputs that are prebound with immobilized DNA circuit elements are only released when the DNA circuit is completed by input DNA. These labeled outputs are released by a fluidic timer to be captured by hybridization for visual detection. Fluidic timers are made from dried sugar; time delays are adjustable from seconds to an hour.

FIG. 43 shows the length of paper-based fluidic delays as a function of percentage of dried sugar according to some embodiments. (A) Delays are created by dipping strips into sugar solutions with different concentrations, followed by drying; delays from minutes to over an hour are possible. A relative delay of 10 equates to approximately 300 seconds for the strips used here. (B) Sugar solutions pipetted onto a paper strip create delays used to stage fluids in different reaction zones.

### DETAILED DESCRIPTION

Systems or “logic circuits” for detecting a biomarker of interest and methods for their use are provided herein. According to the embodiments described herein, such systems may include components which may be used in a nucleic acid strand displacement reaction.

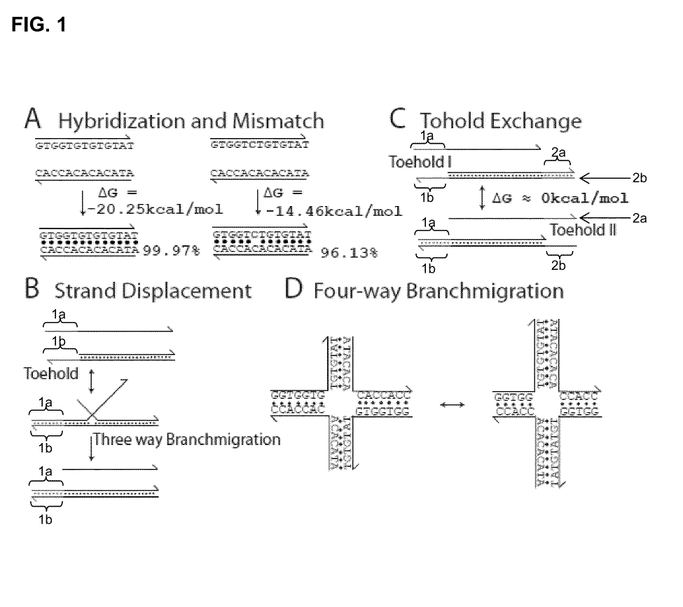

Strand displacement is a process through which two strands with partial or full complementarity hybridize to each other, displacing one or more prehybridized strands in the process. Precise and predictable binding between nucleic acid base pairs allows strands of different lengths to hybridize as shown in FIG. 1A. Energy gained from forming base pairs provides a driving force for hybridization. Substrates that are primarily double-stranded but have one or more single-stranded portions are used in many systems involving nucleic acid hybridization assays. The single-stranded regions, also known as toeholds, hold potential energy for hybridization and provide energy to drive reactions desired direction (FIG. 1B). There can be multiple toe-holds on one strand with different driving directions. The open (or unbound) and closed (or bound) states between toeholds can be exchanged through strand displacement. This strand displacement or exchange process is also known as toehold exchange (FIG. 1C). DNA strand displacement has been used to control logic and kinetics of DNA systems (Zhang & Seelig 2011a). Toehold exchange may be used to identify a single stranded or double stranded biomarker of interest, which are referred to herein as “single stranded toehold exchange” (FIG. 1B-1C) and “double stranded toehold exchange” (FIG. 1D), respectively.

Single Stranded Toehold Exchange/Strand Displacement Systems and Methods for their Use

According to some embodiments, the strand displacement systems described herein are single-stranded toehold exchange systems which include a first catalyst molecule (also referred to as “catalyst strand,” “catalyst molecule” or “input molecule”). As described herein, a catalyst molecule is a single stranded nucleic acid molecule. In some embodiments, the catalyst molecule includes the nucleic acid sequence of a target biomarker of interest that, when detected, indicates the presence of a biologically significant or pathogenic process. In other embodiments, the catalyst molecule acts as an aptamer that binds to or is otherwise conjugated to an amino-acid based biomarker of interest (e.g., peptide, protein, antibody or fragment thereof) that, when detected, indicates the presence of a biologically significant or pathogenic process.

Biologically significant or pathogenic processes that may be indicated by the nucleic acid or amino acid-based biomarker include conditions or diseases that are associated with, for example, a gene mutation, one or more single nucleotide polymorphisms (SNPs), a specific strain of microorganism (e.g., viral, bacteria or fungal strains), a specific member of a family of closely related nucleic acid molecules (e.g., miRNA families), or expression of a particular protein or variant protein. In another aspect, an individual's response or predicted response to therapeutic intervention may be dependent on the presence or absence of the target biomarker. For example, in one embodiment, systems may be used to detect biomarkers associated with the M. tuberculosis complex and with resistance of tuberculosis (TB) to common antibiotics. In some aspects, the system may be used to diagnose TB antibiotic resistance to multiple drugs including rifampicin, isonizid, and kanamycin.

According to the embodiments described herein, the systems described herein may be used in methods for detecting a biomarker of interest. In addition, the systems and methods described below may be used to distinguish between molecules that differ by one or more nucleotides. Such methods may include a step of contacting the first catalyst molecule—which is present in or suspected of being present in a biological sample—with a nucleic acid gate molecule. Upon coming in contact with each other, the first nucleic acid catalyst binds the nucleic acid gate molecule.

In some embodiments, the nucleic acid gate molecule (also referred to herein as a “gate,” a “gate molecule” a “seesaw gate” or a “gate/sensor complex.”), is a double stranded nucleic acid molecule. The gate molecule may include a toehold strand and an output strand (also referred to herein as an “output molecule.”). In certain embodiments, a portion of the toehold strand includes a short nucleotide sequence called a “toehold sequence” or a toehold domain, which is complementary to a portion of the first catalyst molecule, called a toehold binding domain (together, “toehold domains”). In some aspects, the toehold binding domain is approximately 4-15 nucleotides in length, but may be any suitable length to accomplish strand displacement according to the embodiments described herein. The toehold binding domain is what allows binding between the first catalyst molecule and the gate molecule. Upon binding the toehold domain of the gate molecule, the first catalyst molecule initiates strand displacement of the output strand, releasing an output molecule from the gate molecule and forming a gate-catalyst complex.

The output molecule is a single stranded molecule that is released from the nucleic acid gate molecule through strand displacement that occurs when the first nucleic acid catalyst molecule binds to the nucleic acid gate molecule. As used herein, the output molecule may be referred to as the “output strand.” In certain embodiments, the release of the output molecule may result in the output molecule initiating strand displacement in a downstream reaction.

According to some embodiments, the output molecule produces a detectable signal upon release. The detectable signal may be any suitable signal sufficient to visualize or otherwise appreciate relative or definite amounts of the output molecule. In certain embodiments, the output molecule may be conjugated to a detection moiety, which, upon release of the output molecule, may emit a detectable signal. Such a detection moiety may include, but is not limited to, a colorimetric signal, a chemiluminescent signal (e.g., a fluorescent signal, a phosphofluorescent signal, or a luminescent signal), an electrochemiluminescent signal, or an electrochemical signal. In some embodiments, the detectable signal is a fluorescent signal. For example, in one embodiment, the nucleic acid gate molecule may include an output molecule that includes a fluorophore (e.g., FITC, Dig, GFP, YFP, RFP, xanthene derivatives such as fluorescein, rhodamine, eosin, Oregon green, Texas red; cyanine derivatives such as cyanine, indocarbocyanine, and thiacarbocyanine; naphthaleve derivatives; coumarin derivaives; oxadiazole derivatives; pyrene derivatives, oxaxine derivatives, arylmethine derivatives; and Tetrapyrrole derivatives) and a toehold strand that includes a quencher (e.g., using FRET or FET, a dexter electron transfer, chloride, iodide, acrylamide, rhodamine; or a dark quencher such as DABSYL, Qxt quenchers, Iowa black FQ or RQ, IRDye QC-1. In an intact gate molecule, the fluorophore of the output molecule is quenched by the toehold strand. Upon release of the output molecule, the fluorophore and quencher are separated, resulting in a detectable fluorescent signal by the output molecule. In another embodiment, the release of the output molecule results in the output molecule binding to a detection moiety or reporter complex, which results in a detectable signal. A reporter complex may be a double stranded nucleic acid and may contain a fluorophore and quencher located on separate nucleic acid strands. In one embodiment, the reporter complex contains one strand that is complimentary to a domain of the output molecule. Binding of the output molecule to the reporter complex results in strand displacement of one of the strands of the reporter complex, causing separation of stands containing the fluorophore and quencher, which produces a detectable signal. In other embodiments, the detection moiety may be a gold nanoparticle embedded with a nucleic acid or antibody that is complementary to the output molecule.

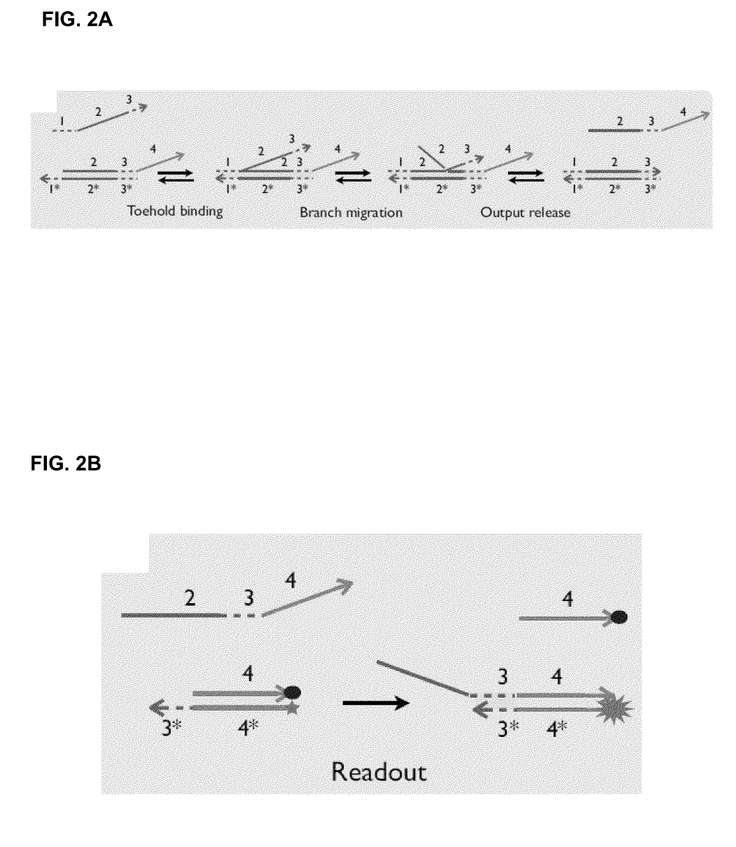

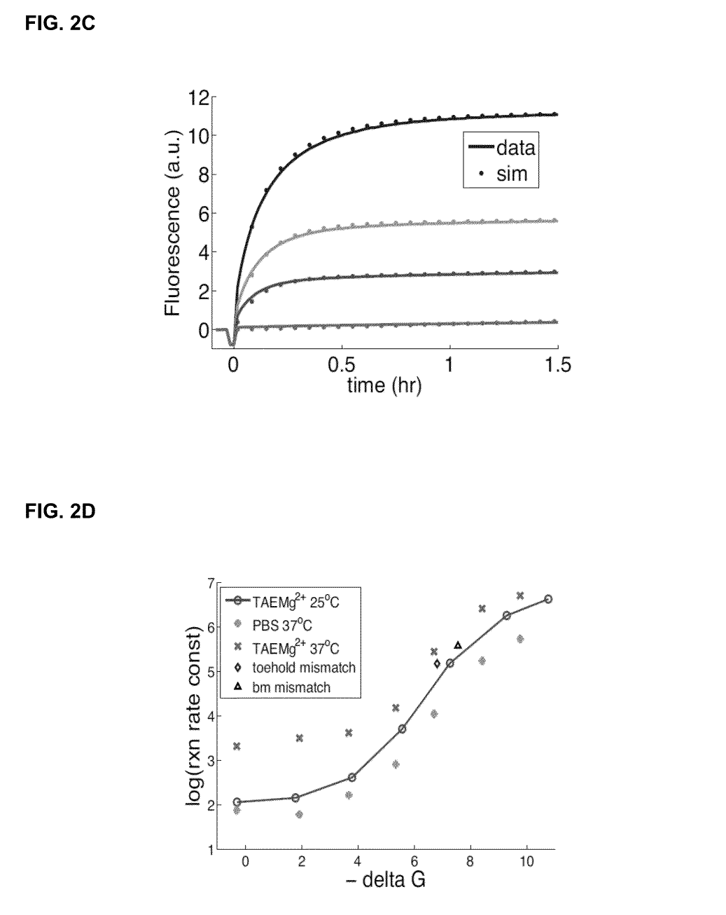

An example of a strand displacement reaction is shown in FIG. 2A. In this example, the functional portions or domains of each nucleic acid strand, are represented by numbers. A starred domain denotes a domain complementary in sequence to the domain without a star (e.g. domain 2* is complementary to domain 2) and complementary domains hybridize to each other via Watson-Crick base pairing (see FIG. 2A). The reaction is initiated when the two complementary toehold domains 1 and 1* bind to each other. In the subsequent random walk process, domain 2 of strand 2:3:4 competes with and is partially displaced by domain 2 of strand 1:2:3. The final step is the complete release of the initial binding partner, i.e. the separation of toehold domains 3 and 3*. The progress of strand displacement reactions is typically assayed using fluorescence (see FIG. 2B). Strand displacement releases at least one single-stranded nucleic acid product or output. In a DNA strand displacement cascade, this output serves as the input to a downstream reaction (see FIG. 2B). This mechanism enables autonomous signal propagation and thus makes it possible to connect individual components into multi-layered reaction networks (Seelig et al., 2006; Soloveichik et al., 2010).

It has been observed that the rate of strand displacement reactions can be quantitatively controlled over several orders of magnitude by varying the strength (length and sequence composition) of toeholds (see FIG. 2D) (Yurke et al., 2003; Li et al., 2002; Zhang et al., 2009). This feature enables engineering control over the kinetics of synthetic DNA devices.

Strand displacement cascades can also be used for catalytic signal amplification. Signal amplification is an important ingredient for many biosensing applications and is sometimes necessary for sensing low-concentration analytes. In one embodiment, strand displacement-based mechanisms are utilized for isothermal signal amplification.

Fuel Molecules Enhance or Amplify Signal Output of a Strand Displacement System

In some embodiments, the system may include a nucleic acid fuel molecule that acts to bind and release the first nucleic acid catalyst from the gate-catalyst complex, allowing the first nucleic acid catalyst to be reused and initiate additional cycles of amplification. In this manner, a biological sample which has a very small quantity of the biomarker can be processed.

In a basic strand displacement reaction, the single-stranded catalyst or input is consumed in the course of the reaction, ending up in an inert double-stranded by-product (see FIG. 2A). In some embodiments, mechanisms may be used through which the same catalytic input molecule can participate in multiple strand displacement reaction cycles, thereby facilitating the release of many outputs and enabling signal amplification. The input can be thought of as acting catalytically, even if no covalent bonds are made or broken.

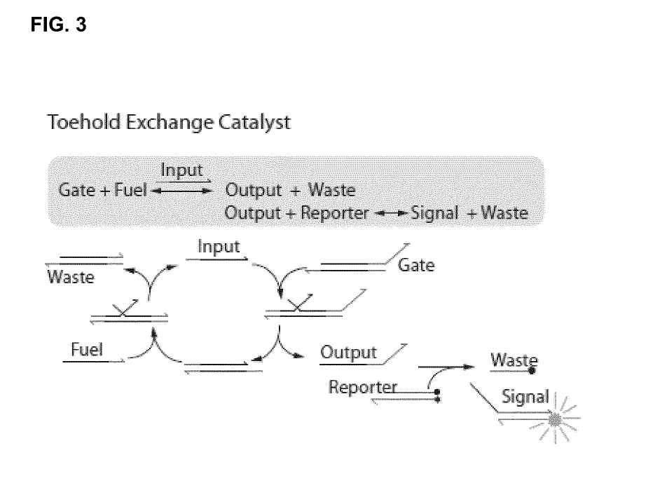

The reactants (other than the catalyst) of these non-covalent DNA catalysis systems generally include DNA strands or complexes that are kinetically trapped in metastable configurations, which act as “fuels” because they collectively store the energy that thermodynamically drives the catalyzed reaction forward (FIG. 3). Interaction between the catalyst and these fuels (via strand displacement) opens a fast pathway for the rearrangement of the fuels into products. The products can yield a fluorescence signal for detection, can lead to DNA nanostructure formation, or can be inputs for downstream strand displacement reactions.

This approach has been explored by demonstrating a system in which the hybridization of two complementary strands was slowed by constraining one or both of the strands via hybridization to shorter auxiliary strands (Tuberfield et al., 2003). A specific input strand could controllably reverse this constraint, and catalytically accelerate the formation of the double-stranded product. Increasingly sensitive and fast amplification mechanisms related to this approach have been demonstrated (Tuberfield et al., 2003; Bois et al., 2005; Green et al., 2006; Seelig et al., 2006; Zhang et al., 2007; Yin et al., 2008; Zhang et al., 2010; Qian et al., 2011).

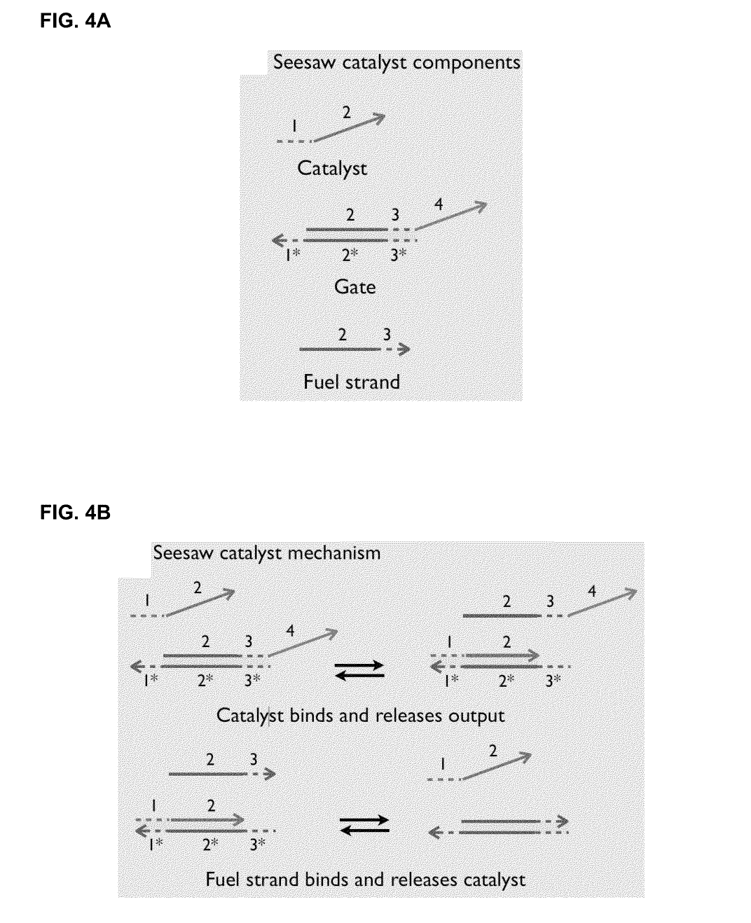

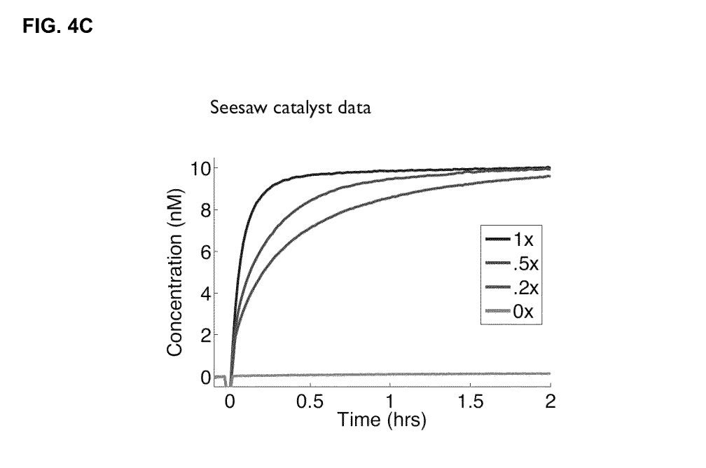

An example of a strand displacement system that includes a fuel molecule is shown in FIG. 4. This system includes a “catalyst” molecule and an “amplifier” to enhance or amplify the output signal (FIG. 4A). The amplifier includes two components, a double stranded “gate” complex and an auxiliary single strand, the “fuel” strand (FIG. 4A). The catalyst acts to initiate the interaction by binding the gate complex through a toehold interaction, which results in the catalytic release of an output molecule from each gate (FIG. 4B, top reaction). The fuel strand then binds and releases the catalyst, allowing the catalyst to interact with another gate molecule (FIG. 4B, bottom reaction). This cascade mechanism allows for the catalyst to sequentially interact with many gate complexes. Each reaction uses up one fuel strand and one gate complex (FIG. 4B).

Sink Molecules Enhance Specificity and Accuracy of Strand Displacement Systems

The systems detailed above describe strand displacement systems and methods for their use that detect differences or mismatches in nucleic acid strands by measuring the kinetics of strand displacement (FIGS. 2C and D, and FIG. 4C). However, one of the major limitations with measuring changes using kinetics is that it is sometimes difficult to reliably distinguish between catalytic molecules that differ by one or more nucleotides (a “mismatched catalyst strand”). This is because a hybridization-based probe will have a high affinity to both a perfectly complementary and mismatched strand. For example, although a mismatch of a single base normally causes about a 4 kcal/mol energy penalty, the energy gain from hybridization of forming other base pairs normally override the small penalty caused by the mismatch except at the melting temperature. In the example shown in FIG. 1A, the energy penalty of the mismatch is 5.79 kcal/mol. Under room temperature conditions, in a 1M sodium salt concentration with 1 uM of each single strand, 99.7% of the product would be a double stranded product without a mismatch and 96.13% of the product would have a mismatch. This small difference is hard to distinguish. To achieve high specificity, the reaction would need to be performed at melting temperature, which varies between sequences and experimental conditions.

Further, the difference between the signals produced by various catalysts may be observable during the early stages of amplification but not at the endpoint because all reactions eventually continue to completion. Additionally, it is difficult to distinguish an output signal for a target catalyst molecule from that of a mismatched catalyst strand without knowing the concentration of each, because the kinetics trace from a mismatch catalyst at a high concentration may appear similar to the kinetics trace from a target catalyst without a mismatch at a lower concentration. Furthermore, measuring reaction kinetics typically requires a sophisticated readout and an expensive instrument, while endpoint detection can be cheaper and can be accomplished much more simply. Thus, a system used to detect a biomarker of interest is needed that is capable of robustly distinguishing sequences that differ by one or more single nucleotide. Additionally, such a system should provide inexpensive and easy-to-use technology for testing in low resource settings.

Thus, in accordance with the embodiments described herein, the systems described above also include a nucleic acid sink molecule (also referred to herein as a “sink molecule”), which acts as a competitive gate molecule, or a targeted molecular “sink.” The sink molecule is a double stranded nucleic acid molecule that includes a competitive toehold strand that is fully complementary to a toehold binding domain of a putative second nucleic acid catalyst (also referred to as a “second catalyst,” or a “mismatched” strand or catalyst). In some embodiments, the putative second nucleic acid catalyst includes a nucleic acid molecule that is related to and/or similar to the first nucleic acid catalyst, but differs from the first nucleic acid catalyst molecule by at least one nucleotide. For example, the second nucleic acid catalyst molecule may be a mutant version of the first catalyst molecule that contains one or more SNPs, a different strain of a microorganism, an additional or alternative member of a nucleic acid family, or a nucleic acid aptamer that binds a mutated or alternate version of a protein or peptide biomarker. The nucleic acid sink molecule acts as a competitive binding substrate for the gate molecule because the second nucleic acid catalyst binds the sink with higher affinity than the gate. In this way, the sink acts to sequester the putative second nucleic acid catalyst as a result of the tight binding, thereby suppressing or preventing an unwanted output signal generated by the second catalyst molecule. According to embodiments described herein, a system that includes a sink molecule for detecting a biomarker of interest allows for robust discrimination of catalyst strands that differ by at least one nucleotide.

The methods may also include a step of sequestering a second nucleic acid catalyst molecule with a nucleic acid sink molecule, wherein the second nucleic acid catalyst differs from the first nucleic acid catalyst molecule by at least one nucleic acid. The method may also include a step of detecting the release of an output molecule.

Biomarkers of Interest

The systems and methods described above may be used to detect any wild type or variant biomarker of interest. In some embodiments, the target biomarker is a nucleic acid which includes, but is not limited to, DNA, such as genomic DNA, cellular DNA, acellular DNA, microorganismal DNA (e.g., bacterial DNA, viral DNA, fungal DNA, yeast DNA); and RNA biomarkers such as messenger RNA (mRNA), transfer RNA (tRNA), ribosomal RNA (rRNA) and ribozymes, viral RNA, small nuclear RNA (snRNA), microRNA (miRNA) and miRNA precursors, small interfering RNA (siRNA) and other regulatory RNA molecules (e.g., piRNA, IncRNA, etc.).

A nucleic acid biomarker may be single or double stranded. In one embodiment, molecular sensors and circuits may be built for the autonomous and embedded analysis of complex molecular mixtures. In some embodiments, these molecular sensors and circuits are used to detect and analyze single stranded catalysts or inputs such as wild type or variant miRNA or mRNA. Expression levels of microRNAs and messenger RNAs as well as deletions, insertions or mutations within messenger RNA sequences can be highly indicative of a disease state (see Sidransky et al., 2002; Weinberg et al., 2002; Cahn et al., 2006; Esquela-Kerscher et al., 2006; Alvarez-Garcia et al., 2005 for examples of cancer markers, all of which are hereby incorporated by reference as if fully set forth herein). Circulating viral RNAs (Tsang et al., 2007, which is hereby incorporated by reference as if fully set forth herein) or microRNAs (Mitchell et al., 2008; Lawrie et al., 2008; Chen et al., 2008, which are hereby incorporated by reference as if fully set forth herein) found in blood are a promising class of biomarkers because they do not require significant processing from the sample. Further, miRNA have been reported to be highly stable in blood serum and the concentration of specific miRNAs can easily reach levels of about 100,000/μl in blood serum (Mitchell et al., 2008, which is hereby incorporated by reference as if fully set forth herein).

Double-stranded biomarkers may also be detected using the systems and methods described herein. For example, bacterial, viral, or cellular genomic DNA from various biological samples (e.g., blood, plasma, serum, urine, sputum, or other sample as described below) can serve as a catalyst molecule, but may require additional sample preparation. In particular, it may be necessary to separate the two strands in a duplex (e.g. by melting) such that one of two resulting single strands can serve as a catalyst or input to a circuit. This is similar to the processing required in hybridization-based DNA microarrays for the detection of genomic DNA.

Alternatively, as described below with respect to double stranded toehold exchange, mutations or other mismatched double-stranded DNA may be detected by four-way branch migration. Four-way branch migration has been observed in biology during meiosis crossover events. This process was previously modeled (Thompson et al. 1976) and branch migration speed has been characterized (Panyutin & Hsieh 1994). Four-way branch migration may therefore be used as part of a four-way toehold exchange mechanism, which is used to probe double-stranded DNA for SNPs that are important for many biomedical applications (Kim & Misra, 2007). See Example 2 below.

In other embodiments, the catalyst molecule is a nucleic acid aptamer, which acts as an adapter molecule to bind a target non-nucleic acid-based biomarker. In such embodiments, the target biomarker is an amino acid-based biomarker including, but not limited to, small peptides, peptide fragments, proteins, antibodies and antibody fragments (see Navani et al., 2006; Liu et al., 2009).

Strand Displacement Cascades May be Used to Build Multi-Component Circuits.

Strand displacement systems, such as those described herein provide a powerful mechanism for engineering multi-input circuits. Feed-forward digital logic circuits have been built that implement a complete set of logical functions (AND, OR, and NOT) using short oligonucleotides as inputs and outputs. Logical values “0” and “1” are represented by low and high concentrations, respectively. Because inputs and outputs are single-stranded nucleic acids, the gates may be cascaded to create multilayered circuits. Examples of nucleic acid-based logic gates, circuits that include nucleic acid-based logic gates and methods of performing operations with the gates and circuits are detailed in Seelig et al. 2006 and U.S. Pat. No. 7,745,594 to Seelig et al., the subject matter of both of which is hereby incorporated by reference as if fully set forth herein.

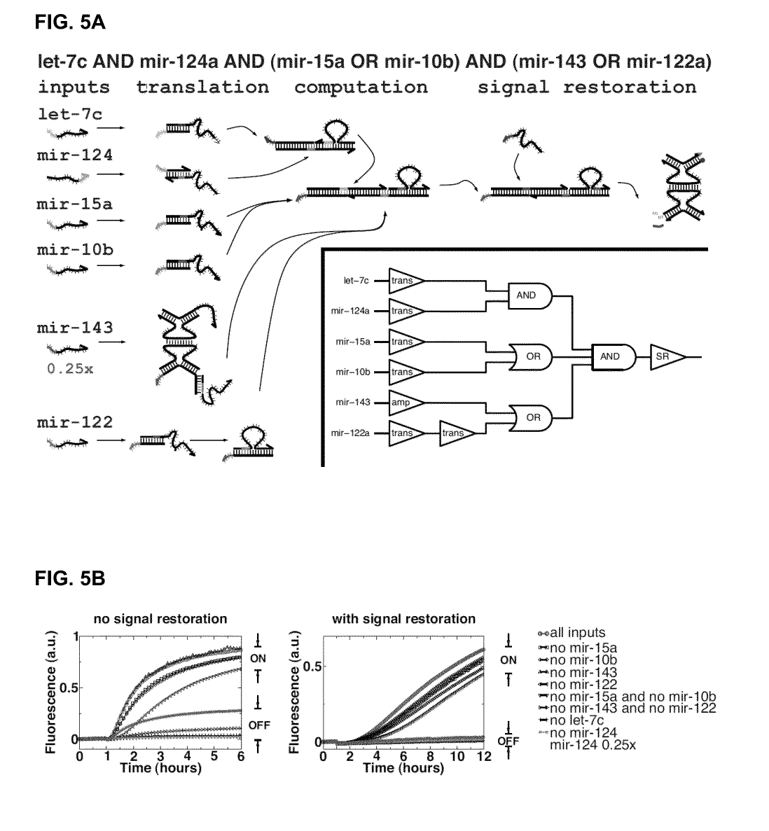

Logic gates work well with both DNA and RNA inputs because gate function relies solely on Watson-Crick complementarity. Additionally, these circuits operate well in the presence of potentially interfering biological RNA at a concentration in excess of gate concentration. Hybridization reactions provide the free energy to move computation forward and Watson-Crick base pairing between modular recognition domains determines the connectivity of gates. The scalability and modularity of the circuit has been shown by combining multiple components into circuits. The largest circuit takes 6 inputs and contains 12 gates in a cascade 5 layers deep (see FIG. 5).

These experiments show that a simple strand displacement reaction mechanism provides the basis for the construction of complex yet reliable molecular circuitry. The size of complexity of strand displacement-based circuits has been expanded (Qian et al., 2011). The DNA logic circuits discussed herein were designed for a situation where inputs can be represented as Boolean variables meaning that they are either present at a very high or very low concentration (Seelig et al., 2006; Qian et al., 2011). However, in many biological classification problems such a Boolean abstraction is an oversimplification. In recent work, it has been shown that it is possible to build DNA logic circuits that can sense and analyze information encoded in the concentrations of multiple analytes, even when not limited to the Boolean approximation (Soloveichik et al., 2010; Zhang et al., 2011).

Double Stranded Toehold Exchange/Strand Displacement Systems and Methods for their Use

According to certain embodiments, the strand displacement systems described herein include double-stranded toehold exchange systems for detection of one or more single base mutations in a double-stranded nucleic acid target (also referred to herein as a “double-stranded target nucleic acid molecule” or “target nucleic acid molecule” or “double stranded target molecule”). In some aspects, a double-stranded toehold exchange system includes two reactants: a detection probe (A) and a double-stranded target nucleic acid molecule (B).

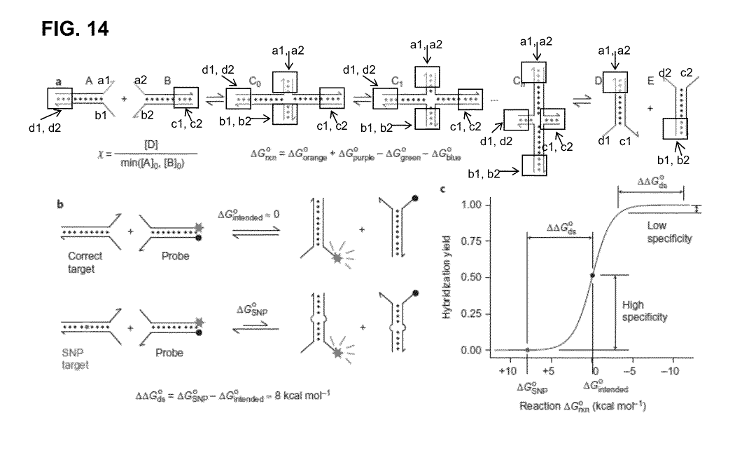

The double-stranded toehold exchange mechanism is shown in FIG. 14a and is modeled in Example 3 (below) according to certain embodiments. Briefly, the reactants (A and B) are two primarily double-stranded nucleic acid molecules; each possessing one or more single-stranded overhangs at the 5′ end of one reactant and at the 3′ end of the other reactant. These overhangs, shown in orange (a1, a2) and purple (b1, b2) in FIG. 14a, are referred to herein as “initiation toeholds”. Toeholds of matching color are complementary to each other, and hybridize to initiate the reaction. The initial four-stranded complex (C₀) then undergoes a ‘branch migration’ process through a series of isoenergetic four-stranded states (C₁ through Cₙ) (Thompson et al. 1976). When the branch migration reaches the other terminus (Cₙ), the dissociation toeholds (blue: c1, c2; and green: d1, d2) spontaneously fall apart to release two primarily double-stranded products (D and E).

The equilibrium concentrations of the various states depend on the length and base compositions of the toeholds, as well as on the length of the homologous branch migration region (shown in black in FIG. 14a; see Example 3 below). In solutions with low concentrations of divalent metal cation, the transition rates between different intermediate C states are high (Panyutin & Hsieh 1994; Panyutin & Hsieh 1993), and the reaction is rate limited by toehold-association and -dissociation processes. With appropriate selection of toehold strengths, both the forward and reverse kinetics are fast, and the reaction rapidly equilibrates from any initial state.

Detection Probe Design.

According to the embodiments described herein, systems and methods for detecting or identifying the presence or absence of one or more single base mutations in a double-stranded target nucleic acid molecule include a detection probe that is designed to hybridize with the double-stranded target nucleic acid molecule in a reaction with proceeds at approximate thermodynamic equilibrium when no single base mutations are present in the double-stranded target nucleic acid molecule (i.e., ΔG°_{intended}≈0). The detection probe may be generated from any suitable nucleic acid molecule including, but not limited to, DNA, RNA, and artificial or modified nucleic acids such as peptide nucleic acids (PNA) and locked nucleic acids (LNA).

In some aspects, the detection probe includes a double-stranded portion (i.e., a “double-stranded probe nucleic acid molecule,” which is also referred to herein as a double-stranded “homologous branch migration region” of the detection probe) that has a first end and a second end, as shown below (x indicates nucleotides that are part of the double-stranded portion of the detection probe according to one embodiment):

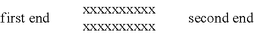

The first end of the double-stranded probe nucleic acid molecule has at least one single-stranded nucleic acid sequence attached or ligated to or extending from its top and/or bottom strand, which is referred to herein as a probe “initiation toehold” or probe “initiation toehold sequence.” In one embodiment, the double-stranded probe nucleic acid molecule has two probe initiation toehold sequences attached or ligated to or extending from its first end of both its top and bottom strand, forming a fork-like structure, which may be referred to herein as a probe “fork toehold” or a probe “initiation double toehold” sequence. A detection probe having an initiation double toehold sequence is shown below (x indicates nucleotides that are part of the double-stranded portion of the detection probe, and i indicates nucleotides that are part of the probe initiation double toeholds according to one embodiment):

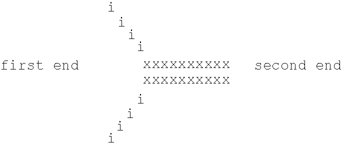

In another embodiment, the double stranded probe nucleic acid molecule has one probe initiation toehold sequence attached or ligated to or extending from the first end of its top or bottom strand, forming a probe “initiation single toehold” or overhang sequence. Detection probes having an initiation single toehold sequence are shown below (x indicates nucleotides that are part of the double-stranded portion of the detection probe, and i indicates nucleotides that are part of the probe initiation single toehold according to some embodiments):

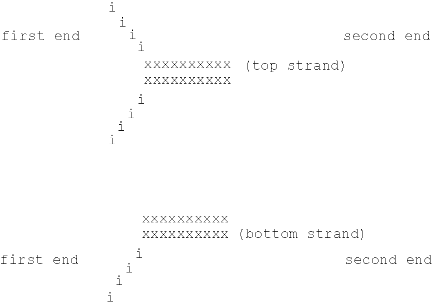

Probe initiation toehold sequence or sequences that may be used in accordance with the embodiments described herein may each be of any suitable length with which to establish favorable conditions including, but not limited to approximately 2 nucleotides (nt) to approximately 10 nt in length. In certain embodiments, the initiation toehold sequence or sequences may be approximately 2 nt, 3 nt, 4 nt, 5 nt, 6 nt, 7 nt, 8 nt, 9 nt, 10 nt, or greater than 10 nt in length. Further, the probe initiation toehold sequences may be attached to, ligated to, annealed to, fused in form with, or otherwise conjugated to the double stranded probe nucleic acid as a separate single-stranded portion or domain of the detection probe, or the probe initiation toehold sequences may be made as part of a single nucleic acid molecule that has multiple portions or domains, e.g., at least one single stranded initiation toehold sequence domain that is adjacent to a double stranded portion or domain.

In some embodiments, the detection probe also optionally includes a double stranded nucleic acid sequence at its second end, which is referred to herein as a probe “dissociation toehold” or probe “dissociation toehold sequence”. The dissociation toehold sequence may be attached or ligated to or extending from its second end. According to some embodiments, when a detection probe includes a probe initiation double toehold, it also includes a dissociation toehold sequence. Such a detection probe is illustrated below (x indicates nucleotides that are part of the double-stranded portion of the detection probe, i indicates nucleotides that are part of the probe initiation double toeholds, and d indicates nucleotides that are part of the probe dissociation toehold according to some embodiments):

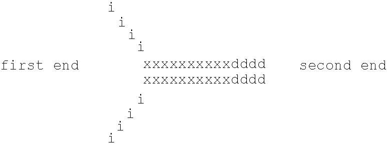

According to other embodiments, when a detection probe includes a probe initiation single toehold, it includes a dissociation toehold sequence only if the double-stranded target nucleic acid does not include a dissociation toehold sequence. In this case, examples of such detection probes are illustrated below (x indicates nucleotides that are part of the double-stranded portion of the detection probe, i indicates nucleotides that are part of the probe initiation double toeholds, and d indicates nucleotides that are part of the probe dissociation toehold according to some embodiments):

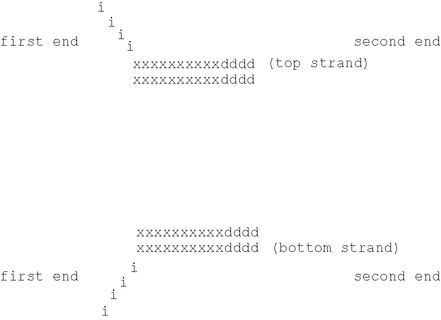

Probe dissociation toehold sequences that may be used in accordance with the embodiments described herein may be of any suitable length with which to establish favorable conditions including, but not limited to approximately 2 nucleotides (nt) to approximately 10 nt in length for each strand. In certain embodiments, each strand of the dissociation toehold sequence may be approximately 2 nt, 3 nt, 4 nt, 5 nt, 6 nt, 7 nt, 8 nt, 9 nt, 10 nt, or greater than 10 nt in length. Further, the probe dissociation toehold sequences may be attached to, ligated to, annealed to, fused in form with, or otherwise conjugated to the double stranded probe nucleic acid as a separate double-stranded portion or domain of the detection probe, or the probe dissociation toehold sequences may be made as part of a single nucleic acid molecule that has multiple portions or domains, e.g., at least one single stranded initiation toehold sequence domain that is adjacent to a double stranded domain that includes a dissociation toehold sequence portion.

According to some embodiments, the detection probe includes at least one substance that is capable of emitting a detectable signal. The at least one substance may be conjugated to or otherwise attached to the second end of the double-stranded probe nucleic acid molecule, and which is capable of emitting or producing a detectable signal upon reacting or hybridizing with a double stranded target nucleic acid molecule. The detectable signal may be any suitable signal sufficient to visualize or otherwise appreciate relative or definite amounts of the products of a reaction between the detection probe and a double stranded target nucleic acid molecule. Examples of substances that are capable of emitting or producing a detectable signal in accordance with the system and methods described herein may include, but are not limited to, a colorimetric signal, a chemiluminescent signal (e.g., a fluorescent signal, a phosphofluorescent signal, or a luminescent signal), an electrochemiluminescent signal, or an electrochemical signal. In some embodiments, the detectable signal is a fluorescent signal. For example, substances capable of emitting a detectable signal that may be attached or conjugated to the second end of the double-stranded probe nucleic acid molecule may include, but are not limited to a fluorophore (e.g., FITC, Dig, GFP, YFP, RFP, xanthene derivatives such as fluorescein, rhodamine, eosin, Oregon green, Texas red; cyanine derivatives such as cyanine, indocarbocyanine, and thiacarbocyanine; naphthaleve derivatives; coumarin derivaives; oxadiazole derivatives; pyrene derivatives, oxaxine derivatives, arylmethine derivatives; and Tetrapyrrole derivatives) and a toehold strand that includes a quencher (e.g., using FRET or FET, a dexter electron transfer, chloride, iodide, acrylamide, rhodamine; or a dark quencher such as DABSYL, Qxt quenchers, Iowa black FQ or RQ, IRDye QC-1. In an intact detection probe, the fluorophore of the top or first strand is quenched by the second or bottom strand. Upon reacting the detection probe with a double stranded target nucleic acid, the fluorophore and quencher are separated to form the products, resulting in a detectable fluorescent signal by the product molecule which includes the fluorophore (see FIG. 14b). Examples of detection probes having at a single or double initiation toehold sequence, a dissociation toehold sequence, a fluorophore, and a quencher are illustrated below (x indicates nucleotides that are part of the double-stranded portion of the detection probe, i indicates nucleotides that are part of the probe initiation double toeholds, d indicates nucleotides that are part of the probe dissociation toehold, P indicates a fluorophore, and Q indicates a quencher according to some embodiments):

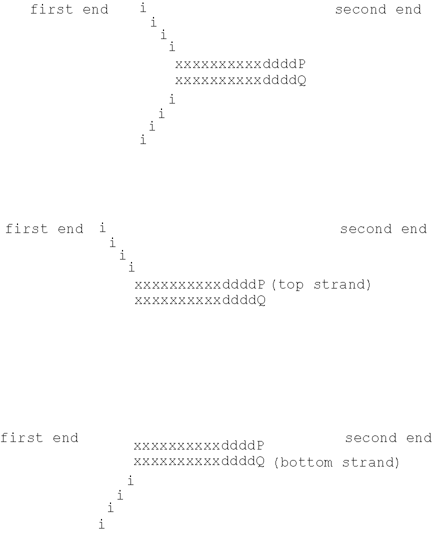

Double-Stranded Target Nucleic Acid Molecules

The systems and methods described herein may be used to determine the presence or absence of one or more single base alteration or mutation (i.e., a point mutation, such as a deletion, insertion, substitution, or single nucleotide polymorphism (SNP)) in a double stranded target nucleic acid molecule. Although the effect of a single base mutation depends on the location of the mutation within a gene, transcript or other nucleic acid molecule, such a mutation can result in the alteration of the expression and/or structure of a protein or peptide. This alteration can have effects that change the function, localization, stability, activation, binding, transcription, translation of a protein, resulting in a pathogenic condition or process including, but not limited to, cancer, cystic fibrosis, sickle cell anemia, and neurofibratosis. A single base alteration may also be of importance in detecting different strains and/or the evolution of new strains of double stranded viruses and bacteria. Therefore, according to certain embodiments, double-stranded target nucleic acid molecules that may be used in such systems and methods include the nucleic acid sequence of a target biomarker of interest that, when a single base mutation is detected, may indicate the presence or absence of a biologically significant or pathogenic process.

Biologically significant or pathogenic processes that may be indicated by a single base alteration or mutation of the target nucleic acid molecule conditions or diseases that are associated with, for example, a gene mutation, one or more single nucleotide polymorphisms (SNPs), a specific strain of microorganism (e.g., viral, bacteria or fungal strains), a specific member of a family of closely related nucleic acid molecules (e.g., miRNA families), or expression of a particular protein or variant protein. In another aspect, an individual's response or predicted response to therapeutic intervention may be dependent on the presence or absence of the target biomarker.

The systems and methods described above may be used to detect any wild type or variant nucleic acid molecule of interest including, but not limited to, DNA, such as genomic DNA, cellular DNA, acellular DNA, microorganismal DNA (e.g., bacterial DNA, viral DNA, fungal DNA, yeast DNA); or RNA such as double stranded RNA molecules found in certain RNA viruses or any RNA molecule hybridized to a DNA or RNA molecule (e.g., an mRNA hybridized to an siRNA, shRNA, InRNA).

According to some embodiments, double stranded target nucleic acid molecules that may be used in accordance with the embodiments described herein have a first end and a second end, wherein at least one target initiation toehold at the first end. The system and methods described herein may be used to detect mutations or alterations of double stranded target nucleic acid molecules wherein each strand is between approximately 5 or more nucleotides and approximately 1000 nucleotides (i.e., between approximately 5 basepairs and approximately 1000 basepairs), between approximately 10 or more nucleotides and approximately 1000 nucleotides, between approximately 20 or more nucleotides and approximately 1000 nucleotides, between approximately 30 or more nucleotides and approximately 1000 nucleotides, between approximately 40 or more nucleotides and approximately 1000 nucleotides, between approximately 50 or more nucleotides and approximately 1000 nucleotides, between approximately 60 or more nucleotides and approximately 1000 nucleotides between approximately 70 or more nucleotides and approximately 1000 nucleotides, between approximately 80 or more nucleotides and approximately 1000 nucleotides, between approximately 90 or more nucleotides and approximately 1000 nucleotides, between approximately 100 or more nucleotides and approximately 1000 nucleotides, between approximately 200 or more nucleotides and approximately 1000 nucleotides, between approximately 300 or more nucleotides and approximately 1000 nucleotides, between approximately 400 or more nucleotides and approximately 1000 nucleotides, between approximately 500 or more nucleotides and approximately 1000 nucleotides, between approximately 600 or more nucleotides and approximately 1000 nucleotides, between approximately 700 or more nucleotides and approximately 1000 nucleotides, between approximately 800 or more nucleotides and approximately 1000 nucleotides, or between approximately 900 or more nucleotides and approximately 1000 nucleotides. In some embodiments, the system and methods described herein may be used to detect mutations or alterations of double stranded target nucleic acid molecules wherein each strand is approximately 5 or more nucleotides, 10 or more nucleotides, 20 or more nucleotides, 30 or more nucleotides, 40 or more nucleotides, 50 or more nucleotides, 60 or more nucleotides, 70 or more nucleotides, 80 or more nucleotides, 90 or more nucleotides, 100 or more nucleotides, 200 or more nucleotides, 300 or more nucleotides, 400 or more nucleotides, 500 or more nucleotides, 600 or more nucleotides, 700 or more nucleotides, 800 or more nucleotides, 900 or more nucleotides, or 1000 or more nucleotides.

In some embodiments, the first end of the double-stranded target nucleic acid molecule has at least one single-stranded nucleic acid sequence attached or ligated to or extending from its top and/or bottom strand, which is referred to herein as a target “initiation toehold” or target “initiation toehold sequence.” In one embodiment, the double-stranded target nucleic acid molecule has two target initiation toehold sequences attached or ligated to or extending from its first end of both its top and bottom strand, forming a fork-like structure, which may be referred to herein as a target “fork toehold” or a target “initiation double toehold” sequence. A target nucleic acid molecule having an initiation double toehold sequence is shown below (n indicates nucleotides that are part of the double-stranded portion of the double stranded target nucleic acid, and t indicates nucleotides that are part of the target initiation double toeholds according to one embodiment):

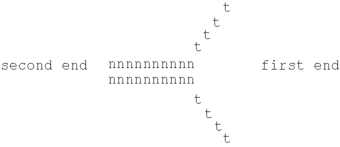

In another embodiment, the double stranded target nucleic acid molecule has one target initiation toehold sequence attached or ligated to or extending from the first end of its top or bottom strand, forming a target “initiation single toehold” or overhang sequence. Target nucleic acids having an initiation single toehold sequence are shown below (n indicates nucleotides that are part of the double-stranded portion of the double stranded target nucleic acid, and t indicates nucleotides that are part of the target initiation double toeholds according to one embodiment):

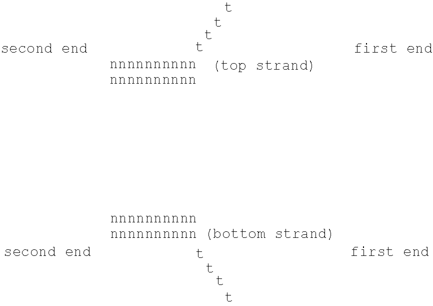

Target initiation toehold sequence or sequences that may be used in accordance with the embodiments described herein may each be of any suitable length with which to establish favorable conditions including, but not limited to approximately 2 nucleotides (nt) to approximately 10 nt in length. In certain embodiments, the target initiation toehold sequence or sequences may be approximately 2 nt, 3 nt, 4 nt, 5 nt, 6 nt, 7 nt, 8 nt, 9 nt, 10 nt, or greater than 10 nt in length. Further, the target initiation toehold sequences may be attached to, ligated to, annealed to, fused in form with, or otherwise conjugated to the double stranded probe nucleic acid as a separate single-stranded portion or domain of the detection probe; or the target initiation toehold sequences may be part of the target nucleic acid molecule, wherein the first end of the target nucleic acid is separated by melting or other suitable process.

In some embodiments, the double stranded target nucleic acid molecule also optionally includes a double stranded nucleic acid sequence at its second end, which is referred to herein as a target “dissociation toehold” or probe “dissociation toehold sequence”. The target dissociation toehold sequence may be attached or ligated to or extending from its second end. According to some embodiments, when a double stranded target nucleic acid molecule includes a target initiation double toehold, it also includes a dissociation toehold sequence. Such a detection probe is illustrated below (n indicates nucleotides that are part of the double-stranded portion of the double stranded target nucleic acid, t indicates nucleotides that are part of the target initiation double toeholds, and a indicates nucleotides that are part of the target dissociation toehold according to one embodiment):

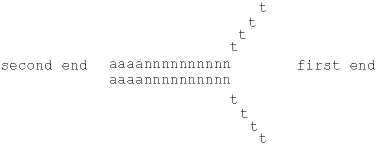

According to other embodiments, when a double stranded target nucleic acid molecule includes a target initiation single toehold, it includes a dissociation toehold sequence only if the detection probe does not include a dissociation toehold sequence. In such a case, examples of such detection probes are illustrated below (n indicates nucleotides that are part of the double-stranded portion of the double stranded target nucleic acid, t indicates nucleotides that are part of the target initiation double toeholds, and a indicates nucleotides that are part of the target dissociation toehold according to one embodiment):

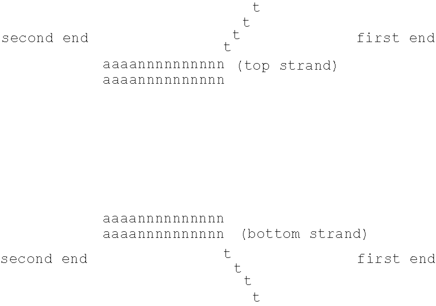

Target dissociation toehold sequences that may be used in accordance with the embodiments described herein may be of any suitable length with which to establish favorable conditions including, but not limited to, approximately 2 nucleotides (nt) to approximately 10 nt in length for each strand. In certain embodiments, each strand of the target dissociation toehold sequence may be approximately 2 nt, 3 nt, 4 nt, 5 nt, 6 nt, 7 nt, 8 nt, 9 nt, 10 nt, or greater than 10 nt in length. Further, the target initiation toehold sequences may be attached to, ligated to, annealed to, fused in form with, or otherwise conjugated to the double stranded probe nucleic acid as a separate single-stranded portion or domain of the detection probe; or the target initiation toehold sequences may be part of the target nucleic acid molecule, wherein the first end of the target nucleic acid is separated by melting or other suitable process.

According to certain embodiments, the target initiation toehold(s) are complementary to the probe initiation toehold(s) such that (i) a top strand target initiation toehold binds and hybridizes to a top strand probe initiation toehold, (ii) a bottom strand target initiation toehold binds and hybridizes to a bottom strand probe initiation toehold, or (iii) both. The binding of the probe and target initiation toehold(s) initiates the double stranded toehold exchange reaction described above. Examples of these interactions are shown below:

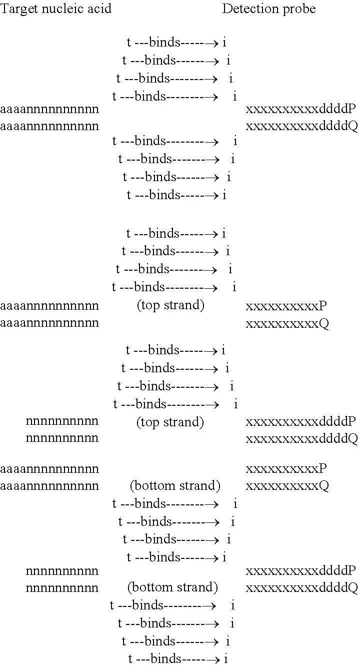

As described in detail below, the detection probe is designed such that when the detection probe hybridizes with the double stranded target nucleic acid as described above, the reaction proceeds at approximately thermodynamic equilibrium, i.e., where ΔG°≈0 when no single base mutations or alterations are present. However, the reaction proceeds at ΔG°<0 when one or more single base mutations or alterations are present. In some embodiments, the ΔG° is proportional to the number of mutations present, and is sensitive to the type of mutation present.

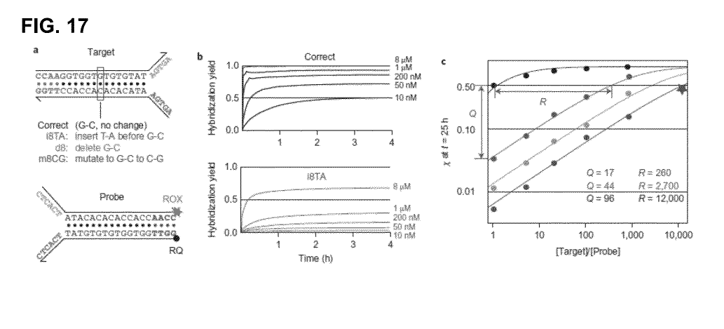

To adapt the double-stranded toehold exchange mechanism into highly specific molecular probes for a dsDNA target nucleic acid molecule (FIG. 14b) in accordance with certain embodiments, the initiation and dissociation toeholds were designed to be similar in length and sequence so that the reaction with the intended target has ΔG°_{intended}≈0. The reaction with SNP targets will have ΔG°_{SNP}≈+8 kcal mol⁻¹ (SantaLucia & Hicks 2004) because of the formation of the two mismatch bubbles (FIG. 17b). The probes discriminate intended targets from SNP targets by taking advantage of the fact that small ΔG° changes near ΔG°=0 have disproportionately large effects on the hybridization yield (x). As ΔG°_{intended} is designed to be approximately 0, a favorable balance is achieved between the binding yield of the intended target (roughly 50%) and the specificity (within a constant factor of optimal specificity (FIG. 17c and Example 3 below)). Thus, the probe is designed to bind specifically to one particular sequence; a spurious or altered molecule that differs by even a single base pair from the intended target, regardless of position within the duplex, exhibits significantly lower binding at equilibrium.

At the opposite end of the initiation toeholds, the probe is functionalized with a fluorophore on one strand and a quencher on the other strand; the close proximity of the fluorophore and quencher ensures that the probe is natively in a dark state. On completion of the double-stranded toehold exchange, the fluorophore and quencher are no longer co-localized on the same molecule, and fluorescence increases. Although fluorescence was used to measure directly the hybridization yield in the studies described in the examples below, probes using any suitable substance that is capable of emitting a detectable signal (such as those described above) may be designed by one skilled in the art in accordance with the embodiments described herein.

To implement the desired ΔG°_{intended}≈0 criterion, the initiation toeholds (orange: a1, a2; and purple: b1, b2; FIG. 14a) were designed to form five base pairs each, and the dissociation toeholds (blue: c1, c2; and green: d1, d2; FIG. 14a) to form four base pairs each. The dissociation toeholds are shorter than the initiation toeholds because the interaction between the fluorophore and quencher stabilizes the reactants (Marras et al. 2002) (see Example 3 below for discussion on toehold lengths). It is noted, however, that the embodiments described herein may include any suitable length of initiation and dissociation toeholds as described above.

Methods of Use

In accordance with the embodiments described above, methods of identifying the presence or absence of one or more single base mutations or alterations are provided herein. Such methods may include a step of reacting a detection probe with a double-stranded nucleic acid target molecule to produce a reaction product which produces a detectable signal. Detection probes and double-stranded nucleic acid target molecules that may be used in accordance with the embodiments described herein may include, but are not limited to, those detection probes and double-stranded nucleic acid target molecules described above and in the Examples below. The double-stranded target nucleic acid molecule may be derived from or obtained from any biological sample from a subject (e.g., a human subject; any other mammalian subject such as dogs, cats, rats, mice, rabbits, guinea pigs, cattle, horses or pigs; or any avian subject), cell line colony, or other research medium, which has a putative double-stranded biomarker of interest. For example, a human biological sample (e.g., blood, plasma, serum, bone marrow, cerebrospinal fluid, urine, tumor tissue, or other relevant tissue sample) may be obtained from a subject to determine whether that subject has (i) a gene mutation that is indicative of cancer or another disease associated with a single base mutation; or (ii) a particular viral or bacterial strain. Further, livestock may be tested to determine if they carry any heritable genetic disease or deformity or any human-transmittable viruses.

The methods for identifying the presence or absence of one or more single base mutations or alterations may also include a step of measuring a level of the detectable signal of the reaction product. The detectable signal may be measured at any suitable time after the reaction is initiated. For example, the detectable signal may be measured anywhere from approximately 1 minute after the reaction is initiated to approximately the time at which the reaction equilibrium is reached. In some embodiments, the detectable signal may be measured approximately 1 minute, approximately 5 minutes, approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 60 minutes, approximately 65 minutes, approximately 70 minutes, approximately 75 minutes, approximately 80 minutes, approximately 85 minutes, approximately 90 minutes, over 90 minutes after the reaction is initiated. In certain embodiments, the signal is measured at the reaction equilibrium.

One skilled in the art may measure the detectable signal by any suitable means based on the type of signal present. For example, if the detectable signal is fluorescence, the signal may be measured by any suitable method of measuring fluorescence including, but not limited to methods using fluorescence spectroscopy, fluorescence microscopy, fluorometer, immunologic methods, fluorescence resonance energy transfer (FRET), biosensors, fluorescence-activated cell sorting (FACS), ethidium bromide, and species altered fluorescence imaging (SAFI).

The methods for identifying the presence or absence of one or more single base mutations or alterations may also include a step of determining a target hybridization yield (x) via the measured level of detectable signal. These determinations are described in detail in Examples 3 and 4 below. According to some embodiments, the hybridization yield may be used to determine whether a single base mutation (e.g., a SNP) is present based on a threshold hybridization yield that distinguishes a target nucleic acid having a mutation or alteration from a target nucleic acid that does not have a mutation or alteration. The threshold may be as low as approximately 5% to 10% or up to approximately 50%, depending on the time at which the detectable signal is measured (i.e., when the signal is measured after the reaction is initiated). When a target hybridization yield is more than approximately 5%, more than approximately 10%, more than approximately 20%, more than approximately 30%, more than approximately 40%, or is approximately 50%, this indicates the absence of mutations or alteration in the double stranded target nucleic acid molecule. However, a target hybridization yield is that is less than approximately 50%, less than approximately 40%, less than approximately 30%, less than approximately 20%, less than approximately 10%, or less than approximately 5%, this indicates the presence of one or more mutations or alterations in the double stranded target nucleic acid molecule.

Methods for Detecting a Biomarker of Interest Using a Clinically Relevant Diagnostic Tool.

In some embodiments, DNA-based biosensing circuits and systems, such as those described above, may be designed that can autonomously analyze and interpret the information encoded in a set of molecular disease markers. The use of molecular algorithms for biosensing is most promising in situations where advanced laboratory instrumentation is not available. Thus, in one embodiment, systems including synthetic molecular circuitry may be integrated with diagnostic tools (e.g., paper-based lateral flow devices) to generate easy-to-use diagnostic tests for low resource settings (LRSs). This approach allows multi-analyte testing in LRSs by providing simplified actionable readout of complex test results without the need for an instrument. As such, the systems and methods described above may be used for detecting a biomarker of interest in a biological sample. According to some embodiments, such methods may include a step of exposing a biological sample which contains or is suspected of containing a first nucleic acid catalyst molecule that includes a biomarker of interest to a diagnostic tool.

According to the embodiments described herein, biological samples that may contain or be suspected of containing the biomarker of interest include in vitro and in vivo biological samples. In vitro biological samples that may be used in accordance with the methods described herein may include, but is not limited to, cultured cells (e.g., cells with membrane-bound biomarkers or cultured cell lysates) or cell culture supernatant. In vivo biological samples that may be used in accordance with the methods described herein may include, but are not limited to, whole blood, serum, plasma, blood cells, urine, sputum, saliva, stool, spinal fluid or CSF, lymph fluid, the external secretions of the skin, respiratory, intestinal, and genitourinary tracts, tears, milk, neuronal tissue, lung tissue, any human organ or tissue, including any tumor or normal tissue, any sample obtained by lavage (for example of the bronchial system or of the breast ductal system), and also samples of ex vivo cell culture constituents. The sample can optionally be diluted with a suitable eluant before performing a diagnostic assay.

Thus, in one embodiment, the systems described above may be embedded in or otherwise part of a paper-based diagnostic tool, such as a lateral flow device. These devices may include a two-stage process of logic circuit reaction followed by target capture and detection. Paper networks were recently developed that include embedded timing mechanisms that allow programming of multi-step fluidic processes in simple paper devices. The concept arose from a history developing instrumented microfluidic cards for point-of-care diagnostics. For example, card-based assays were developed for detection of antigens (malaria) and IgM antibodies (measles, dengue, typhoid, Rickettsia) for differential diagnosis of infections that share the symptom of rapid-onset high fever (Yager et al., 2008; Yager et al., 2006). The assays were carried out on a nitrocellulose membranes using dry gold detection reagents stored on-card. The purpose of the card and instrument was to carry out the processing

In some embodiments, the biological sample may be exposed to the diagnostic device without additional processing. However, in some embodiments, the biological sample may undergo further processing to isolate, enrich, and/or amplify the nucleic acid in the sample. Such processing methods are known in the art and are commercially available (e.g., Qiagen, Sigma Aldrich, Promega, Invitrogen, Norgen Biotek Corp,), and include, but are not limited to, cell lysis, column-based purification, ethanol precipitation, phenol-chloroform extraction, Trizol extraction.

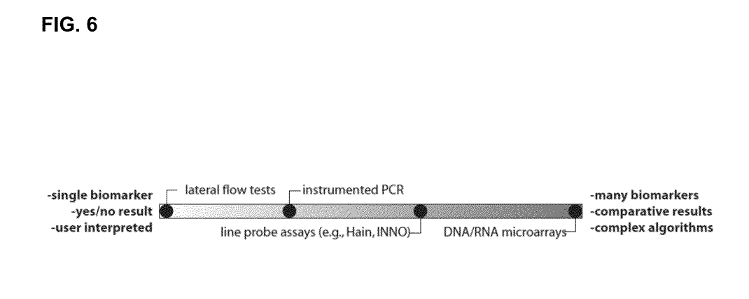

Suitable diagnostic tools that may be used according to the methods described herein include, but are not limited to, microfluidic devices, lateral flow tests (LFTs), instrumented PCR methods, line probe assays (e.g., Hain, INNO), and nucleic acid microarrays. These diagnostic tools vary from very simple tests appropriate for LRSs through very complex tests which are more suited for lab settings (see FIG. 6). Nearly all laboratory assays are based on multiple processing and detection steps carried out in a timed sequence by a technician or a machine. For example, PCR normally requires sample preparation by a trained operator followed by instrumented amplification and detection by a fluorescence reader. The enzyme-linked immunosorbent assay (ELISA) is widely used for detection of proteins, antibodies, or some small molecules; it requires a dozen or more manual steps with timed incubations followed by absorbance measurement by a dedicated instrument. Tests like these are useful when time and facilities are abundant, but not in field or clinical settings where easy-to-use rapid turnaround analytical tests are desired (e.g., infectious disease diagnostics, detection of biowarfare agents, water quality testing) (Peeling et al., 2010; Urdea et al., 2006; Yager et al., 2008; Yager et al., 2006).

According to some embodiments, the diagnostic tool uses a paper-based diagnostic tool that includes a reaction with a visual readout, such as paper-based a microfluidic device or a lateral flow test (LFT) (also referred to as a “lateral flow device”). An LFT diagnostic tool is based on the wicking of a sample through a matrix or other interface, treated with biochemical reagents (e.g., pregnancy tests are LFTs) (Shih et al., 2010). LFTs and other paper-based diagnostic tools provide rapid test results (5-30 minutes), can be run by an untrained user, and allow visual detection of the test result (e.g., pregnancy test lines are gold nanoparticles or latex beads). An LFT diagnostic tool may comprise any suitable material able to wick fluids by capillary action including, but not limited to, paper (e.g., filter paper, chromatography paper), cellulose, cellulose acetate, nitrocellulose, cloth, or a porous polymer film. Nitrocellulose is a widely used paper in LFTs; it is a notable material as a reaction substrate (O'Farrell et al., 2009).

Although LFTs and other paper-based diagnostic tools are typically created from a straight paper strip and used to detect one catalyst molecule, it is appreciated that the LFTs and other paper-based diagnostic tools described herein may include different shaped (e.g., tree-shaped) paper devices that split a sample into discrete zones with different test chemistries (Martinez et al., 2007; Martinez et al., 2008; Dungchai et al., 2010; Li et al., 2010; Wang et al., 2010; Zhao et al., 2008.) or may include more than one reaction and/or detection on a single strip of paper such that each reaction proceeds in series. Such paper devices provide an advantage over conventional LFTs by allowing multiple tests to be performed on a single sample, however they do not aid the user in interpreting the results of multiple tests. Therefore, a test that aids the user in interpreting the results of multiple tests would be useful.

Therefore, in some embodiments, an LFT or other paper-based diagnostic tool includes at least one reaction zone and at least one detection zone, and may include a plurality of reaction and detection zones to visually detect the one or more results of a single or multiple test. The reaction and detection zones contain one or more components of a strand displacement system, such as those described in detail above. In certain embodiments, a reaction zone includes a set of reaction components, which include at least one gate molecule and at least one sink molecule (described in detail above), both of which are attached to the LFT substrate directly (e.g., by drying a solution of reaction components onto the paper matrix) or indirectly (e.g., via beads within the fiber matrix of a paper-based LFT). In certain embodiments, the set of reaction components also includes at least one fuel molecule. When a biological sample that contains or is suspected of containing a biomarker of interest (i.e., a first catalyst molecule) and/or a second (or “mismatched”) catalyst molecule is exposed to a corresponding reaction zone, the first catalyst molecule binds the gate molecule, initiating a strand displacement reaction and releasing an output molecule, as described above. Any suspected mismatched catalyst molecules that are present in the biological sample bind preferentially to the sink molecules, thereby quenching mismatched molecules and preventing a signal output attributable to mismatched molecules.

In some embodiments, the LFT also includes a time delay zone. The time delay may include one or more sugar solutions dried on the LFT that create fluidic time delays that can be controlled by the sugar concentration.

The methods described herein may also include a step of detecting the biomarker of interest by visualizing a change in signal when the output molecule binds a nucleic capture molecule that is attached to a detection zone. In certain embodiments, the one or more detection zone includes output capture probes, which are attached to the LFT directly or indirectly. The output capture probes are complementary to the output strands released in the reaction zone, such that the output strands hybridize to the output capture probes, thereby immobilizing or capturing the output strands within the detection zone.

As described above, the output strand emits an output signal which is visually detectable on an LFT at the detection zone. In some embodiments, the output strand, when released from the gate molecule, emits a fluorescent signal that represents presence of the first catalyst molecule (i.e., the biomarker of interest). Any suitable detection moiety may be conjugated to the output strand, as described in detail above. Alternatively a detection moiety (e.g., gold nanoparticles) may be conjugated to the output capture probe or directly attached to the detection zone of the LFT.

LFTs (or nucleic acid lateral flow, NALF) have been used for visual detection of DNA from culture or DNA that has been amplified in a benchtop instrument (Ngom et al., 2010). Thus, in some embodiments, the output strand may be captured and the output signal visually detected by any method known in the art including, but not limited to, amplicon hybridization to immobilized probes (Edwards et al., 2006; Kalogianni et al., 2011; Corstjens et al., 2003; Carter et al., 2007; Mao et al., 2009; Rule et al., 1996, the subject matter of all of which is hereby incorporated by reference herein, as if fully set forth herein), antibody binding to hapten-tagged amplicons (antibody-dependent) (Kalogianni et al., 2011; Corstjens et al., 2001; Noguera et al., 2011, the subject matter of all of which is hereby incorporated by reference herein, as if fully set forth herein), and streptavidin binding to biotin-tagged amplicons (Corstjens et al., 2003; Corstjens et al., 2001; Noguera et al., 2011, the subject matter of all of which is hereby incorporated by reference herein, as if fully set forth herein). In some embodiments, visible labels, including gold nanoparticles, may be used to detect amplified products. Line probe assays (LiPAs) represent one example of extending this format to parallel detection of multiple markers; e.g., the Hain Genotype MTBDR+ test includes 24 readout lines to identify TB strains and common resistance genes (Abebe et al., 2010; Bang et al., 2006; Brossier et al., 2006; Cavusoglu et al., 2006; Friedrich et al., 2011; Hillemann et al., 2005; Hillemann et al., 2005; Miotto et al., 2006; Parsons et al., 2011). Although LiPAs make it possible to ask complex diagnostic questions, they require careful interpretation by an expert. In one embodiment, a novel diagnostic approach embeds interpretation algorithms in a molecular circuit to provide a simple actionable readout from complex test results. This approach may be applicable to a wide array of analytes and multi-analyte testing algorithms.

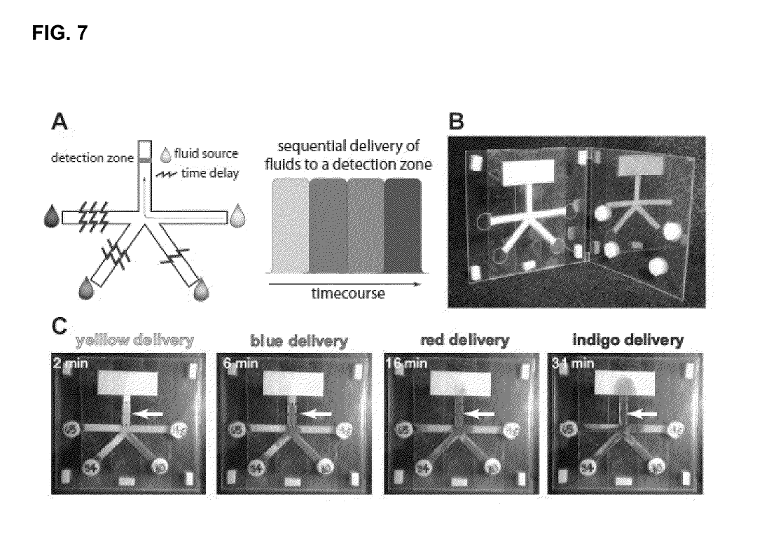

As described above, LFTs are an excellent candidate for point of care diagnostics—they are fast, easy to use, and do not require an instrument. Paper-based LFTs materials have also incorporated automatic volume metering and timing mechanisms based on shaped paper networks (Fu and Lutz et al., 2010; Fu and Ramsey et al., 2010; Kauffman et al., 2010; Osborn et al., 2010), such that assays that require multiple timed steps can be programmed into inexpensive paper devices (see FIG. 7 for one example).

It is widely recognized that eliminating the need for an instrument is an important step to reach settings outside the laboratory, since instruments are not only expensive, but they are more likely to break and less likely to be serviceable (Peeling et al., 2010; Urdea et al., 2006; Yager et al., 2006; Yager et al., 2008). In conventional devices, removing the instrument shifts more burden onto the user because they must perform more steps and must interpret the results, which directly leads to increased potential for user error. The combination of DNA logic circuits and systems with low-cost paper-based diagnostic devices provides a solution to these outstanding challenges in point of care diagnostics.

The following examples are intended to illustrate various embodiments of the invention. As such, the specific embodiments discussed are not to be construed as limitations on the scope of the invention. For example, one of skill in the art will appreciate that the system described herein represents a platform technology and, as such, specific sequences of the system components described herein may be tailored to any chosen biomarker. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1

### DNA Amplifiers with Single Nucleotide Specificity

An amplification system has been developed that can discriminate between single strands of nucleic acid that differ at one or more single nucleotide position. This system may contain a strand without a nucleic acid base mismatch (called a “catalyst”), which can trigger an amplification reaction leading to a strong output signal, while a strand with a single nucleotide difference (called a “mm-catalyst”) does not lead to any discernible signal at the end of the experiment. Distinguishing single-stranded nucleic acids that differ by only a single position is a challenging problem because any hybridization-based probe will have a high affinity to both the perfectly complementary strand and the mismatched strand. The experiments performed in this Example may be performed in solution phase and may be used reliably at room temperature.

Using this system, one DNA or RNA strand containing a specific known mutation may be distinguished from a sequence that does not contain this mutation. Amplification of the target over the mutated sequence using this system may be at least approximately a 100-fold to at least approximately a 1000-fold amplification. This approach may be extended to the more difficult problem of selectively amplifying one or multiple sequences from a family of closely related sequences. For example, the let-7 family of microRNAs may be used as a biologically relevant sample (Roush et al., 2008) as described in more detail below. There are on the order of 10 let-7 family members in humans that differ from each other by only one or two mutations. The let-7 family thus provides an ideal test case for this technology. The assays used are fluorescence-based kinetics experiments (see FIG. 2 for typical reaction conditions).

Results.

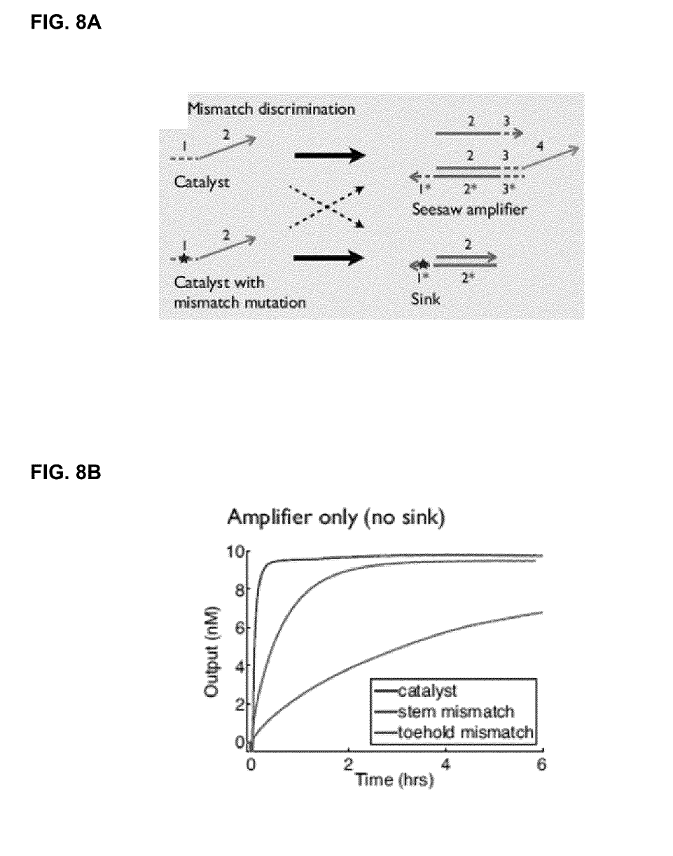

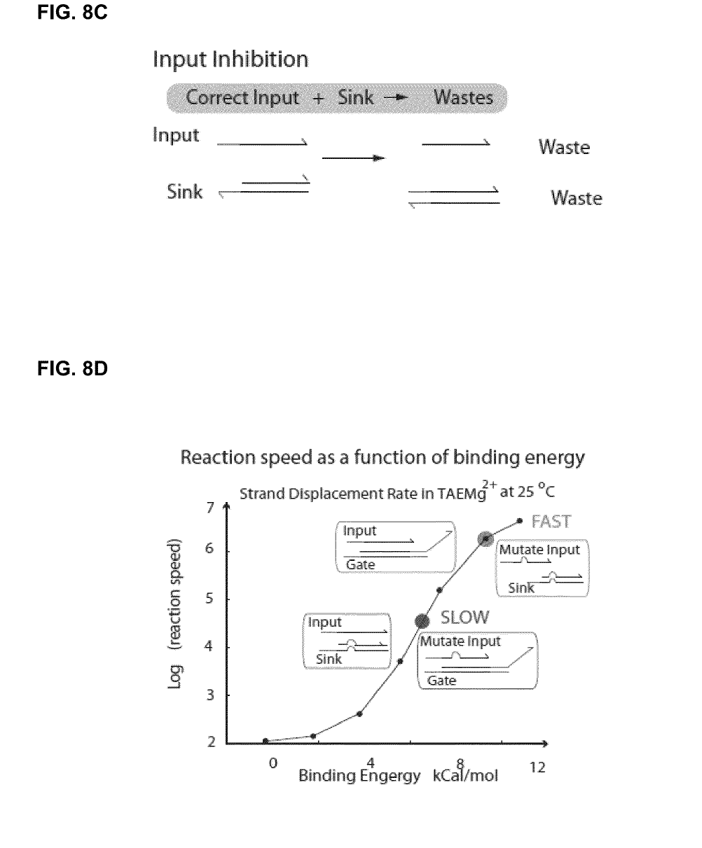

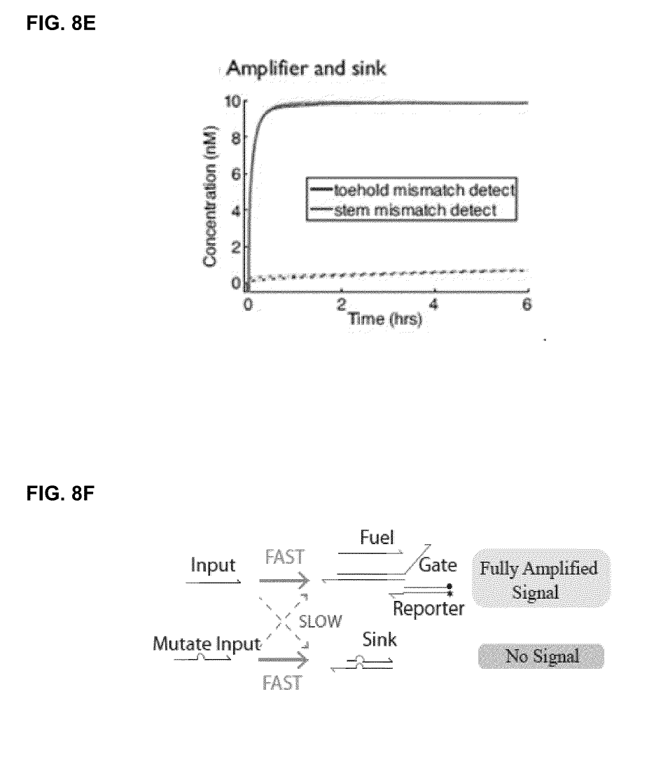

A first mismatch discrimination module is based on the “seesaw” amplifier system (Qian et al, 2011; FIG. 4A; FIG. 8A catalyst and seesaw amplifier only). The kinetics of strand displacement reactions depend strongly on the strength (length and sequence composition) of the toehold, which is an important aspect for this system. Shortening the toehold domain of the input strand (domain 1 in FIG. 2A) by a single base can decrease the reaction rate constant up to about 10-fold; introducing a mismatch in the same toehold domain produces a similar effect (FIG. 2D). (Introducing mismatches in the branch migration domain 2 also leads to observable differences (FIG. 2D); here, the focus is on toehold mismatches). Mismatch discrimination using a single strand displacement reaction was reported previously (Li et al, 2002); however, in this experiment, mismatch discrimination is integrated with amplification.

The experimental data in FIG. 8B shows that a simple seesaw catalytic system is highly sensitive to a single mismatch in the toehold domain of the catalyst strand. However, although reaction kinetics are slowed down considerably relative to a system that contains no mismatch in the input, the difference is only observable in the early stages of amplification and not at the endpoint: both the catalyst and the mm-catalyst eventually turn over all available substrates (fuel+gate) and thus produce the same endpoint signal. Therefore, although the speed of the reaction (strand displacement kinetics) is very sensitive to the change in binding energy, endpoint discrimination is not achieved in this catalytic reaction because the reaction only proceeds in one direction. All correct and mismatched catalysts trigger the reaction, although at different rates, and eventually all go to completion. Thus, it is difficult to distinguish differences between multiple inputs by looking only to the endpoint signal.

Discrimination based on reaction kinetics is unsatisfactory for two reasons. First, knowledge of catalyst concentrations is required. For example, both catalyst and mm-catalyst strands are used at the same concentration in the experiment presented in FIG. 8B. However, increasing the concentration of the mm-catalyst in the reaction could make the corresponding trace look similar to the trace for the catalyst without a mismatch. Consequently, the reaction rate constant will not be accurate unless the concentration of all reactants is known. Second, measuring reaction kinetics requires a sophisticated readout and an expensive instrument, while endpoint detection can be cheaper and accomplished much more simply.

To achieve endpoint discrimination, different destinations were provided for different inputs in an amplification system by adding competitive binding substrates. The system shown in FIG. 8A combines a seesaw catalyst with a competitive threshold gate or “sink.” The sink is a strand displacement gate where the longer strand is fully complementary to the mm-catalyst (FIG. 8C). The mm-catalyst binds with the sink approximately 100 times faster or more than with the seesaw gate; thus, the mm-catalyst is more likely to bind to the sink than it is to interact with the seesaw gate (FIG. 8D). Furthermore, binding to the sink is essentially irreversible while binding to the seesaw gate is reversible by design. Conversely, unlike the mm-catalyst, the catalyst is about 100 times more likely to bind to the seesaw gate. The overall effect is that the mm-catalyst is rapidly bound by the sink and does not produce a signal, while the catalyst binds the seesaw gate, which triggers a catalytic amplification cycle leading to a strong signal (FIG. 8E). By using different gates and sinks, different inputs can “choose” their own path and achieve different endpoint states (FIG. 8F). This reaction mechanism may be further characterized for a wide range of concentrations of all components. Additionally, RNA may also serve as an input in this system.

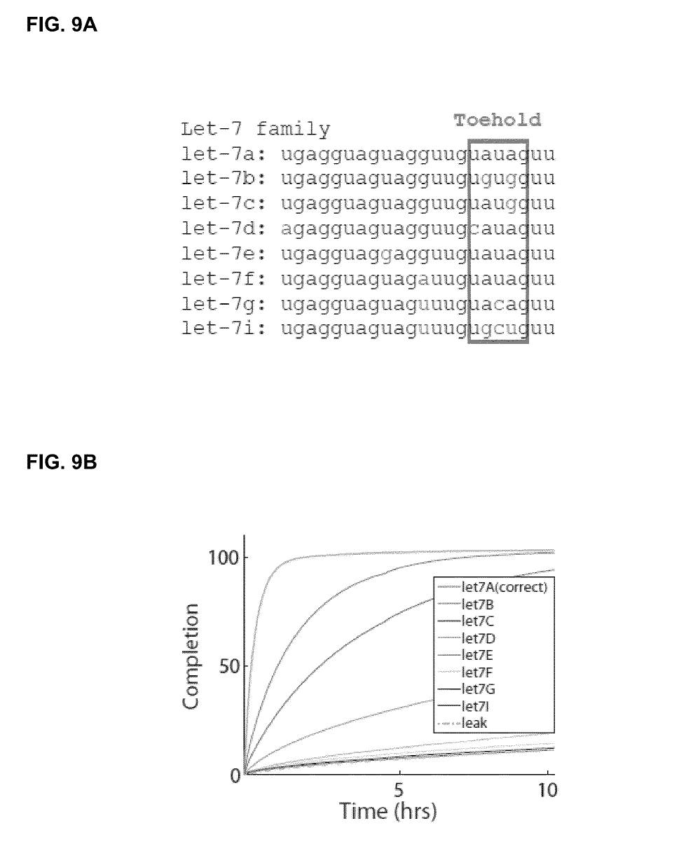

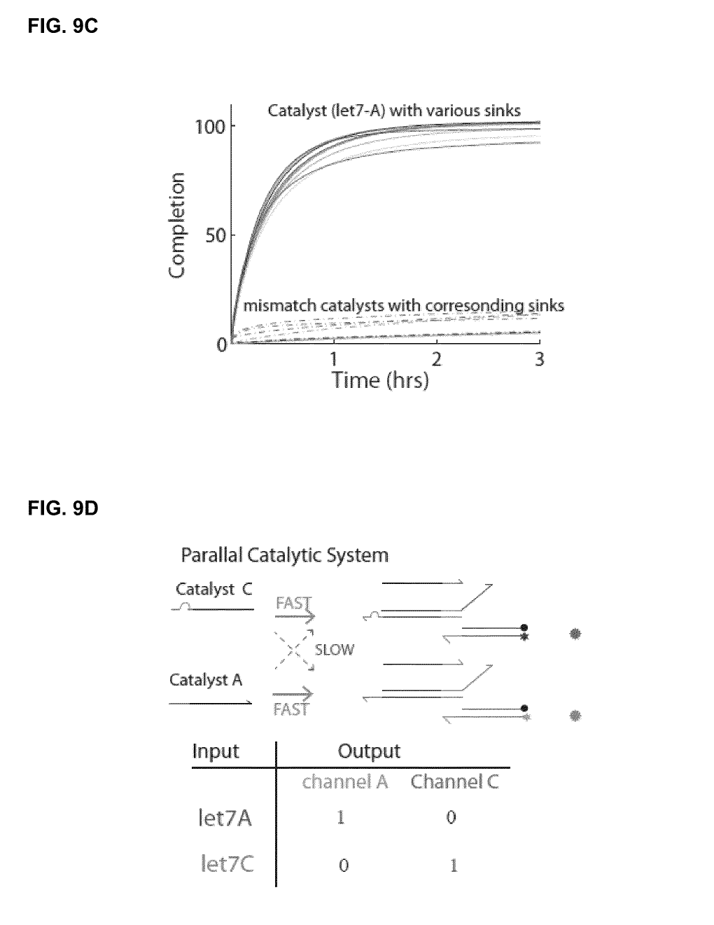

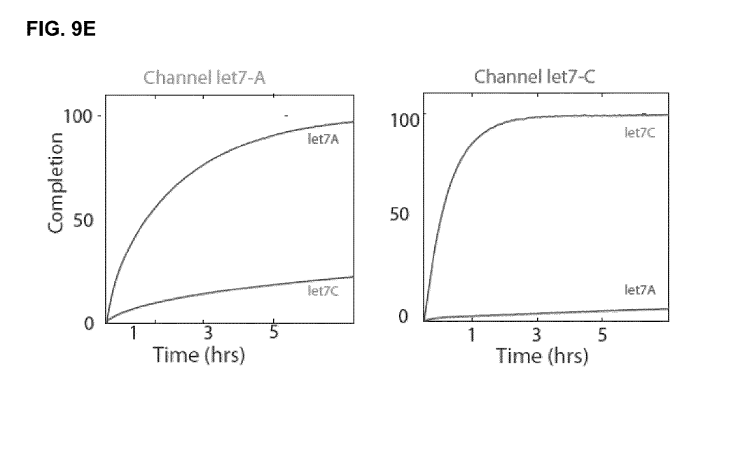

As a further application of this mechanism, an amplification system for the detection of the Lethal-7 (let-7) microRNA family was designed (FIG. 9). The let-7 family is a group of miRNAs with very similar sequences that play an important role in human cancer and can be used to diagnose cancer cells. Eight similar sequences from the let-7 family were tested as shown in FIG. 9A. These sequences are shown below (bolded nucleotides show differences between family members and underlined nucleotides represent toeholds):

let-7a:
(SEQ ID NO: 1)
UGAGGUAGUAGGUUGUAUAGUU
let-7b:
(SEQ ID NO: 2)
UGAGGUAGUAGGUUGUGUGGUU
let-7c:
(SEQ ID NO: 3)
UGAGGUAGUAGGUUGUAUGGUU
let-7d:
(SEQ ID NO: 4)
AGAGGUAGUAGGUUGCAUAGUU
let-7e:
(SEQ ID NO: 5)
UGAGGUAGGAGGUUGUAUAGUU
let-7f:
(SEQ ID NO: 6)
UGAGGUAGUAGAUUGUAUAGUU
let-7g:
(SEQ ID NO: 7)
UGAGGUAGUAGUUUGUACAGUU
let-7i:
(SEQ ID NO: 8)
UGAGGUAGUAGUUUGUGCUGUU

Because DNA is cheaper and more stable than RNA, the system was tested using DNA inputs of the same sequences of the let-7 miRNAs. A system was first built for the explicit detection of let-7A; however, the system can also be triggered by other members of the let-7 family (FIG. 9B). Additionally, when corresponding sinks were added to the system reaction, the reactions containing mismatched inputs produced little signal (FIG. 9C, mismatch catalysts with corresponding sinks) while reactions containing the correct input continued to completion as if there were no sink present (FIG. 9C, catalyst (let7-A) with various sinks). Due to the similarities of DNA with RNA, this system may also be directly tested utilizing an RNA input.

Different paths for different sequences can also be set using a gate instead of a sink. Gates for mismatched inputs can serve as sinks for the correct catalyst. Two catalytic systems were designed for let-7A and let-7C, which contain only one base difference (FIG. 7D). When let-7A was added to the reaction, a fully triggered signal was produced in channel A (FIG. 7E, left panel, blue line) while no signal was produced in channel C (FIG. 7E, right panel, blue line). Whereas, when let-C was added to the reaction, no signal was produced in channel A (FIG. 7E, left panel, green line) while a fully triggered signal was produced in channel A (FIG. 7E, right panel, green line).

Furthermore, there is the potential to generate higher discrimination factors by taking advantage of this catalytic system in which input can be reused. This system may also be built on paper based lateral flow devices (Fu et al., 2010) to make high specificity diagnostic devices that can be used easily everywhere.

It is desirable to increase the sensitivity of strand-displacement based amplifiers to the level that is sufficient for direct detection of biomarkers from biological samples without additional amplification. For example, a two-stage catalytic amplifier cascade that was sensitive to a concentration of 1 pM (or 600,000 molecules/μl) of a synthetic input was previously reported (Zhang et al., 2007). For comparison, another report demonstrated that the concentrations of diagnostic miRNA biomarkers in blood are in the range of 10,000/μl to 100,000/μl (Mitchell et al., 2008). This implies that strand displacement-based amplification circuits are sensitive to concentrations in the range relevant for biosensing applications. The sensitivity also depends strongly on the type of measurement and conditions of the readout. However, it would be desirable to increase sensitivity by at least an order of magnitude.

Gel-based purification may be used for assembly of the amplifier complex in addition to other methods of purification of nucleic acids to enhance the sensitivity. Further, restriction enzymes may be used to process gate components from biological DNA (thus starting from an essentially perfect template) and ligation may be used to assemble long DNA strands from multiple short pieces that can be synthesized with high fidelity.

In addition, modified bases such as 2′O-methyl RNA bases and Locked Nucleic Acid bases may be used as components of nucleic acid amplifiers. Introduction of modified bases may be used to control the thermodynamics and kinetics of reactions involving multi-stranded complexes. Such modifications may help to decrease leak reactions by improving the stability of base pairing at nicks or at helix ends. To increase gain even further, multi-step cascades of fixed gain amplification reactions may be designed. Such cascading enables large gain, even if the gain in each stage is limited.

### Example 2

### Double-Stranded Mutation Detection

Detection of double-stranded DNA is difficult because the DNA is already in a minimum energy state and does not have any reactive single-stranded bases available for binding. In typical experimental conditions (room temperature, non-denature), the time for a double-stranded molecule to spontaneously dissociate can be months to years. Thus, to detect mutations in double-stranded DNA, researchers normally separate the double-stranded DNA into two single-strand molecules by melting the DNA. Various methods have been used to get pure single-stranded DNA from genome. However, long single-stranded nucleic acids often adopt complex secondary structures, in which regions are inhibited from hybridization to the detection probe. To avoid secondary structure, it is desirable to keep the target double-stranded. To do so, a double stranded probe may be generated to take advantage of four-way branch migration. By avoiding secondary structure of dissociated strands, there is no limitation on target sequence. Thus, longer and more efficient probes may be more easily designed.

As a further advantage, an improved discrimination may be obtained using double-stranded probes. Because mutations in double-stranded molecule happen on both sides, there are two mismatched nucleotides in the target double stranded molecule. As such, the resulting energy penalty is doubled compare to a single-stranded detection system.

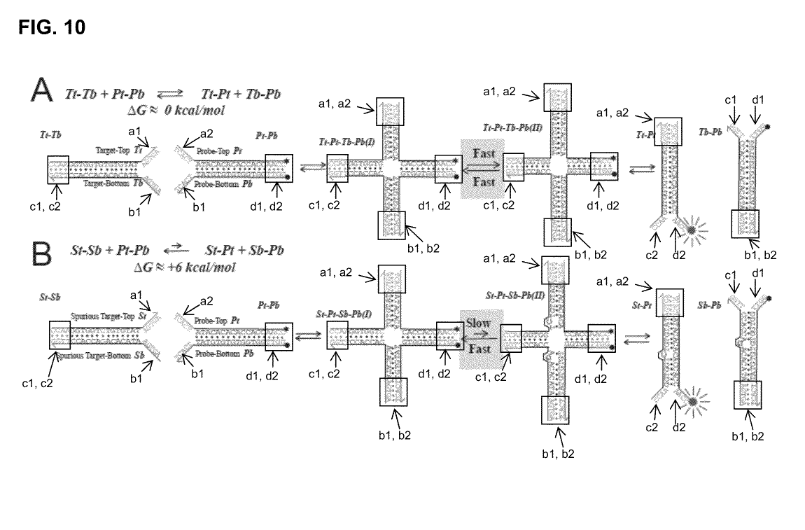

The reaction mechanism is explained in FIG. 10. With the complementary ‘fork’ toehold on Probe and Target (orange: a1, a2; and purple: b1, b2), a Target-Top(Tt) strand hybridizes with a Probe-Top (Pt) strand; and a Target-Bottom (Tb) strand hybridizes with a Probe-Bottom (Pb) strand. This toehold binding event initiates a four-way branch migration. Because the four-way branch migration region has homologous sequences at the junction point (called Holliday junction), each nucleotide on Tt has the same probability of binding its complement on Tb or Pt.

Because each branch migration step has the same probability of proceeding forward and backward, the junction will ultimately reach the toehold (blue: c1, c2; and green: d1, d2) on the opposite end. Spontaneous dissociation of the blue (c1, c2) and green (d1, d2) toehold results in the product TtPt and TbPb, each with exposed (or “open”) blue (c1, c2) and green (d1, d2) toeholds. In addition, the fluorophore and quencher interaction are broken, allowing a fluorophore to be detected. The exposed blue (c1, c2) and green (d1, d2) toeholds on the products may initiate reverse reaction and drive the product to the original state. The strength of all toeholds may be adjusted, such that the forward and backward reactions have similar speed. Fast reaction speed on both directions ensures rapid approach to equilibrium. Using this design, the hybridization yield (X) is calculated as follows:

$$χ = \frac{[ TtPt ]}{{[ Probe ]}_{time = 0}} \approx 0.5$$

In the case of a spurious target (FIG. 10B), once the branch migration reaches the mismatched point, the mutated nucleotide on the Spurious-Top (St) strand is much more likely to bind to the Spurious-Bottom (Sb) strand than to the Probe-Top (Pt) strand, because Sb contains the same mutation that complimentary to the mutation on St. If St binds to Pt, the mutated base will not hybridize with the base on Pt. The formation of each mismatch typically carries an energy penalty approximately 4 kcal/mol. Also, Sb will bind to Pb and has similar energy penalty. Therefore, the binding of the spurious target results in a total free energy that is approximately 8 kcal/mol less favorable than the perfect match. This large energy penalty makes the forward reaction much less favorable than the reverse reaction. As such, few products (StPt and SbPb) are made, such that X<<0.5.

The discrimination factor, Q, is defined as the hybridization yield ratio of the correct target over the mutated target when they have same concentration as the probe:

$$Q = \frac{{χ}_{correct}}{{χ}_{mutated}}$$

Additionally, the concentration tolerance, R, is defined as the ratio of concentrations needed to achieve 50% yield at equilibrium:

$$R = \frac{{[ StSb ]}_{χ = 0.5}}{{[ TtTb ]}_{χ = 0.5}}$$

A large R value ensures specific discrimination over a large range of concentrations.

According to some embodiments, a model can be built to predict sensitivity. The reaction equation of the model may be written as:

PtPb+TtTb [symbol] PtTt+PbTb

and has initial condition:

PtTt=PbTb=0,PtPb=c,TtTb=n·c

At equilibrium, the initial condition is represented as follows:

PtTt=PbTb=x,PtPb=c−x,TtTb=n·c−x.

Thus the equilibrium constant is reflected in the following equation:

$${K}_{eq} = \frac{{x}^{2}}{( c - x ) ( nc - x )}$$

If the hybridization yield is defined as X=x/c, the above equation can be written as:

$${K}_{eq} = \frac{{χ}^{2}}{( 1 - χ ) ( n - χ )} \\ χ = \frac{{K}_{eq} + {K}_{eq} \cdot n - \sqrt{{K}_{eq}} \sqrt{{{K}_{eq} ( n - 1 )}^{2} + 4 n}}{2 ( {K}_{eq} - 1 )} \qquad ( Equation A )$$

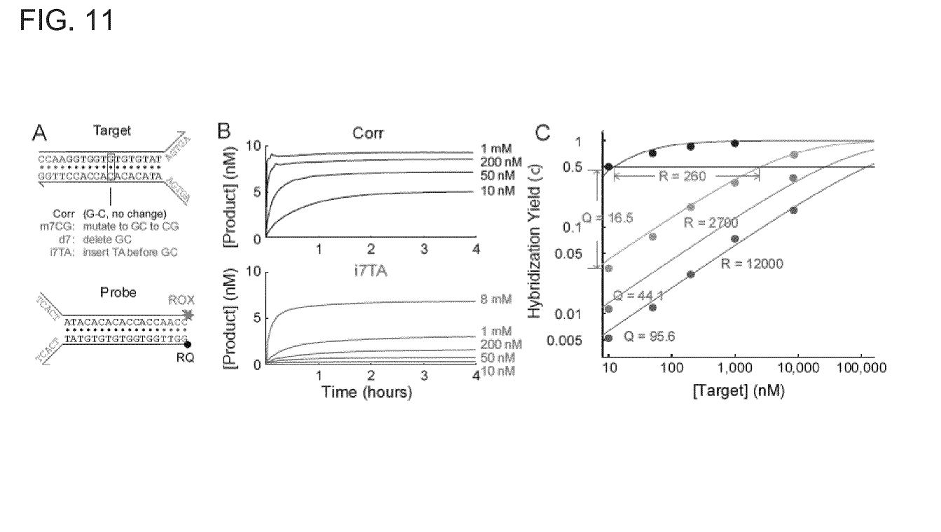

Because K_{eq}=Exp(−ΔG/RT), where ΔG is the free energy of the reaction, R=1.986 cal/mol/K is the gas constant and T=298K is the room temperature. Thus, hybridization yield X can be expressed as a function of the ΔG and n(input concentration), as shown in FIG. 11C (curve). From Equation A, when X is small, then:

x≈\sqrt{K_{eq}·n},

which means that the hybridization yield grows as a square root of input concentration, or, in other words, R≈Q² (Equation B).

Thus, this system should have quadratic tolerance on concentration. To test our prediction, experiment to test a high concentration of mutated target was performed. Using Equation B (R≈Q²), the experimental results agreed with the relationship, as shown in FIG. 11.

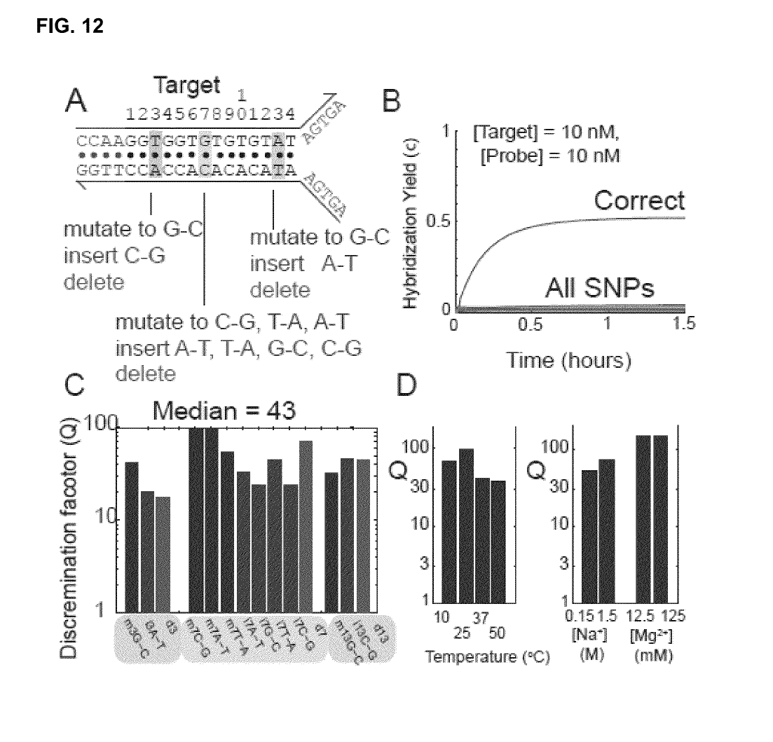

This reaction TtTb+PtPb [symbol] TtPt+TbPb is a bimolecular reaction with two products and has no net gain in base pair binding (ΔH=0), no enthalpy change, a very small ΔG change, thereby ensuring that the system is robust under varied temperatures, concentrations, and salinity. In an experimental study, this system is also robust under conditions of 10° C., 37° C., 50° C., and in Na+ or Mg2+ solution of various concentration. Various mutations with different positions and identities were tested, and produced a median Q of 43 as shown in FIG. 12.

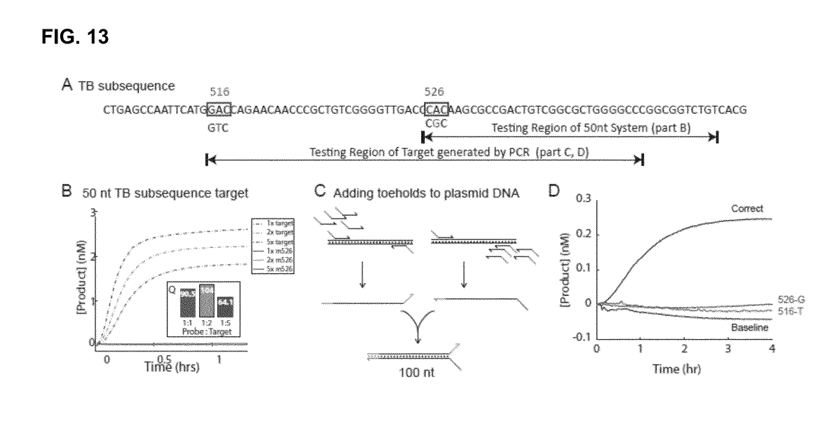

To test robustness over sequence length, experiments were performed on a TB subsequence that is 50 nt in length (CTGAGCCAATTCATGGACCAGAACACCCGCTGTCGGGGTTGACCCACAAGCGCC GACTGTCGGCGCTGGGGCCCGGCGGTCTGTCACG (SEQ ID NO:9)) (FIG. 13A), resulting in a Q≈100 (FIG. 13B). To illustrate a typical detection experiment, detection of a drug resistant marker on tuberculosis (TB) was used. A TB subsequence (100 base pairs in length) was cloned into a plasmid. Besides the wild type, a plasmid with a TB sequence having a mutation at amino acid positions 516 and 526 (FIG. 13A). Experiments were performed to detect the single base differences between three plasmids. The ‘fork’ toeholds may be generated using unbalanced PCR followed by annealing as shown in FIG. 13C. The annealed product was tested and purified using grouse gel and tested with a probe (ordered from IDT, pre-annealed). The kinetics results are shown in FIG. 13D, illustrating that the plasmid with wild type TB sequence was easily distinguished from the mutated TB sequence. This system shows robustness with an unknown concentration and salinity under room temperature and can be used to detect mutation for a random sequence around 100 base pairs.

### Example 3

### Analytical Framework and Probe Design in Double-Stranded Toehold Exchange

### Design Principles for Double-Stranded Toehold Exchange

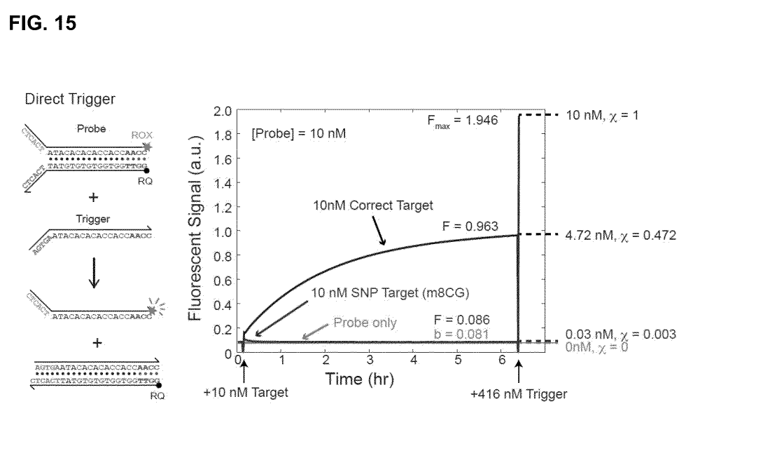

The double-stranded toehold exchange process can be modeled as a series of states connected by transition rates (FIGS. 14A and 15). Intermediate states in which the four strands are co-localized are omitted, but in which the toeholds are incompletely hybridized (FIGS. 14B and 15), because these states suffer both entropic and enthalpic penalties, and consequently will not be prevalent in solution. Intermediate states in which internal base pairs are “breathing” (FIGS. 14C and 15) are likewise omitted. The reduced states of the reaction are thus:

A+B⇄C₀⇄ . . . ⇄Cᵢ⇄ . . . Cₙ⇄D+E (Eq. 1)

In the consideration of the energies of each of these states with respect to determination of occupancy (concentration) at equilibrium, a standard DNA thermodynamic model is assumed in which only Watson-Crick base stacking energies are considered. Furthermore, for simplicity, it is assumed that the energies of all 4-helix multiloops in the various branch migration states Cᵢ are identical in strength. Under these assumptions, the (n+1) different branch migration states Cᵢ are identical in energy, and can thus be grouped into a conglomerate state C with energy:

$$Δ {G}_{C}^{∘} = Δ {G}_{{C}_{1}}^{∘} - RT ln ( n + 1 ) \qquad ( Eq . 2 )$$

Doing so preserves the partition function of the original system in Eq.1:

$$\begin{matrix}Z = {e}^{- Δ {G}_{A + B}^{∘} / RT} + ( {\sum }_{i = 0}^{n} {e}^{- Δ {G}_{{C}_{i}}^{∘} / RT} ) + {e}^{- Δ {G}_{D + E}^{∘} / RT} \\ = {e}^{- Δ {G}_{A + B}^{∘} / RT} + {e}^{l n ( n + 1 )} {e}^{- Δ {G}_{{C}_{i}}^{∘} / RT} + {e}^{- Δ {G}_{D + E}^{∘} / RT} \\ = {e}^{- Δ {G}_{A + B}^{∘} / RT} + {e}^{- ( Δ {G}_{{C}_{i}}^{∘} / RTl n ( n + 1 ) ) / RT} + {e}^{- Δ {G}_{D + E}^{∘} / RT}\end{matrix} \qquad$$

In this reduced reaction system A+B⇄C⇄D+E, the equilibrium concentration of C is low

$$( {e}^{- Δ {G}_{C}^{°} / RT} << Z )$$

when either ΔG°_{C}>ΔG°_{A+B} or ΔG°_{C}>ΔG°_{D+E}. The difference between the values of ΔG°_{A+B} and ΔG°_{C}_{₀} is due to the combined ΔG° values of the orange (a1, a2) and purple (b1, b2) initiating toeholds. Furthermore, to correctly account for the bimolecular A+B state in the Markov model, its ΔG° should be adjusted by the a concentration term RTln(c), where c is the current concentration of the excess species of A and B (scaled to 30M for hybridization initiation entropy). Although c will change with time and progression of the reaction, it is bounded; assuming that [A]₀<[B]₀, [B]₀≧c≧[B]₀−[A]₀.

$$\begin{matrix}Δ {G}_{A + B}^{∘} = Δ {G}_{{C}_{0}}^{∘} - Δ {G}_{toeholds}^{∘} + RT ln ( c ) \\ = Δ {G}_{C}^{∘} + RT ln ( n + 1 ) - Δ {G}_{toeholds}^{∘} + RT ln ( c )\end{matrix}$$

To satisfy ΔG°_{C}>ΔG°_{A+B},

ΔG°_{toeholds}>RT ln(c(n+1))

The conditions for ΔG°_{C}>ΔG°_{D+E} is similar, except using the blue (c1, c2) and green (d1, d2) toehold energies instead.

The equilibrium concentrations of C must be low for the dsDNA probe to function properly. When the equilibrium concentration of the C states are not low, much of the probe could be trapped in intermediates, which not only results in low fluorescence for correct target binding (due to failure to dissociate), but also slow the kinetics of the overall reaction when the probe is trapped in intermediates with the SNP targets.

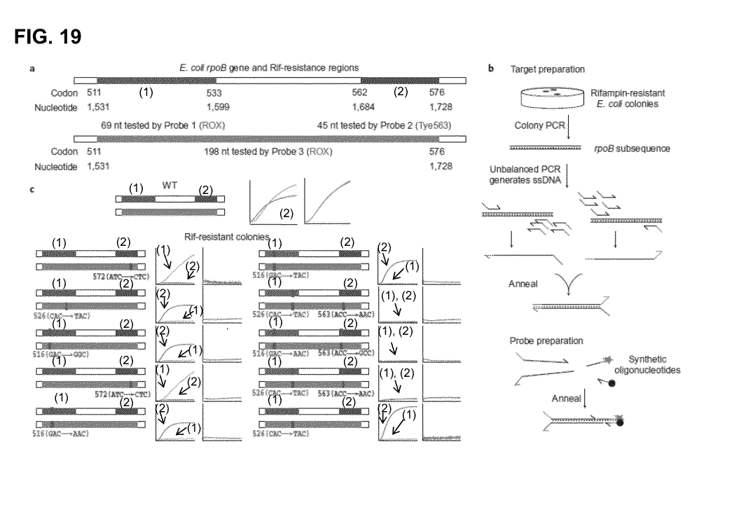

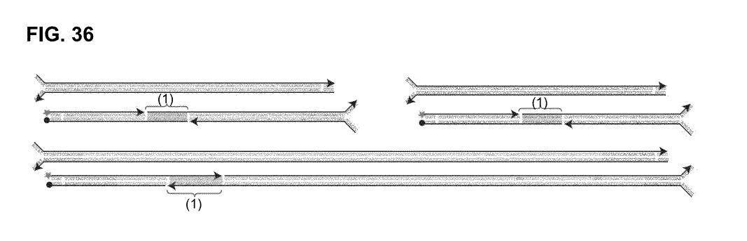

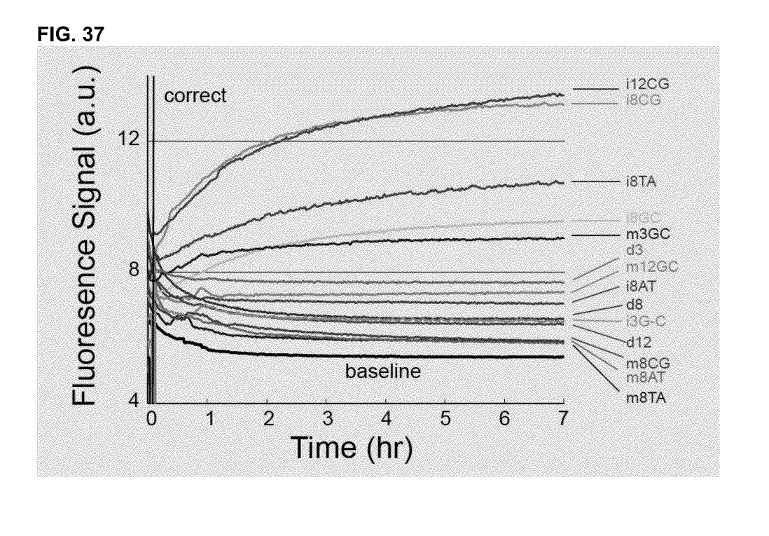

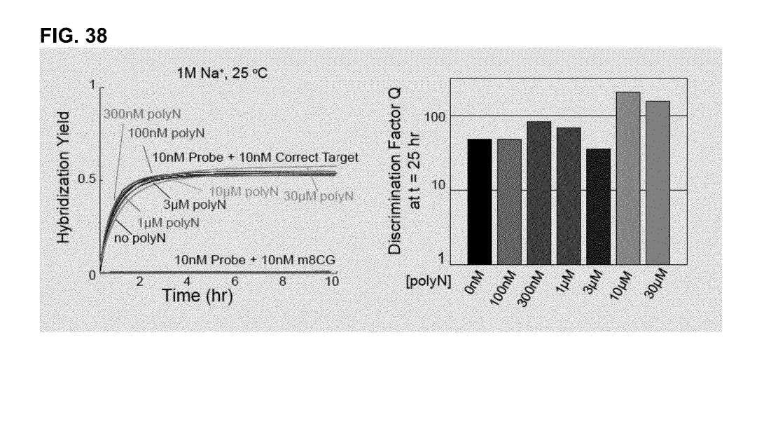

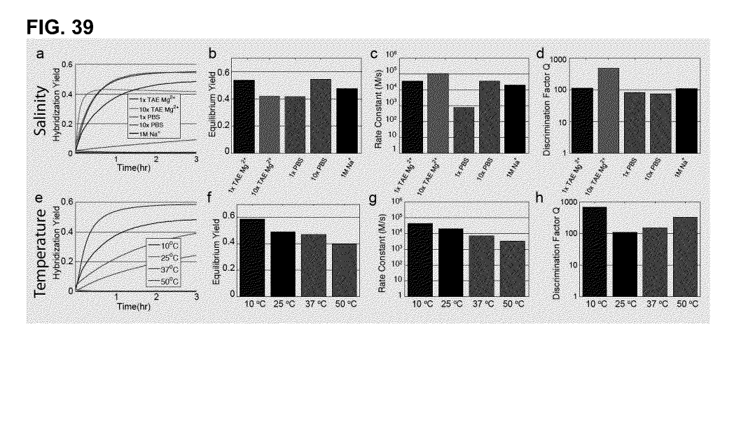

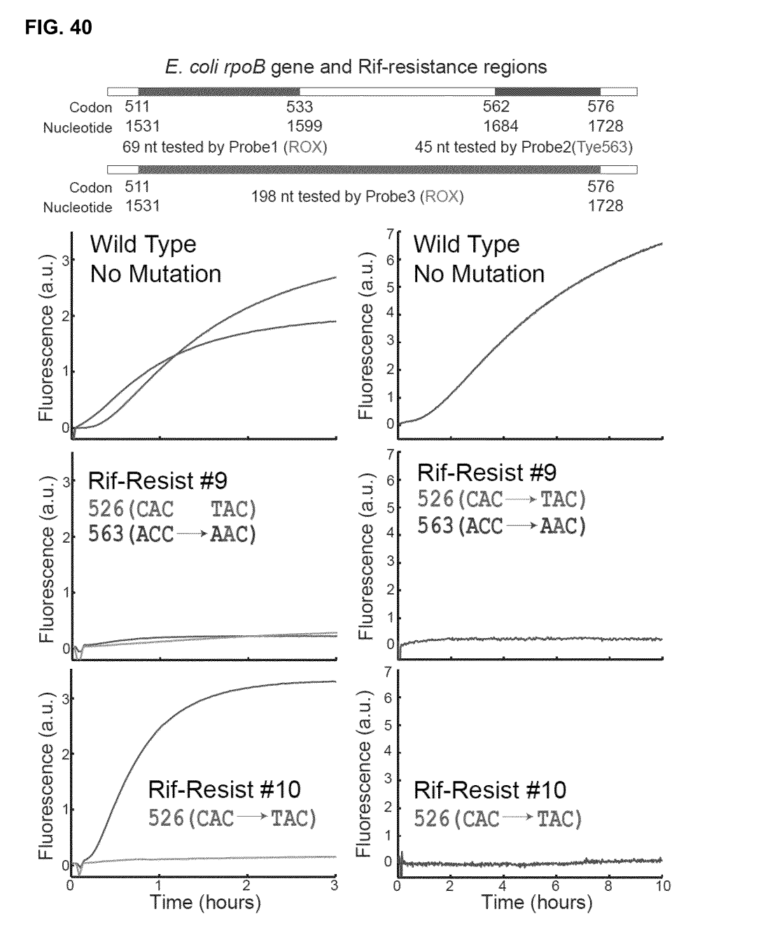

For the experiments described in the Examples below, n varied between 14 and 233, c was typically 10 nM, so RTln(c(n+1)) varied between −9.8 kcal/mol and −11.4 kcal/mol. The blue (c1, c2) and green (d1, d2) toeholds were typically weaker (4 base pairs each), with total ΔG°_{toeholds}=−8.86 kcal/mol, according to established thermodynamic parameters. The interaction between the ROX fluorophore and the Iowa Black RQ quencher likely also contributes to the thermodynamic stability of the toeholds. Consequently, the studies illustrated in FIG. 19 and FIG. 36 that use a 200 nt branch migration region may result in some intermediate state trapping. However, because higher temperatures and lower salinity concentrations tends to weaken the thermodynamic contributions of base paring (making ΔG°_{toeholds} less negative), the probes for very long regions will likely show superior performance at elevated temperatures and reduced salinities.

Conditionally Fluorescent Probes for dsDNA

The high specificity conditionally fluorescent dsDNA probe involves a special case of double-stranded toehold exchange, in which the standard free energy ΔG° of the probe binding to the intended target is approximately 0. This is achieved by designing the initiation and dissociation toeholds to be approximately the same length and strength. The reason that a ΔG°≈0 is chosen is because this gives a good tradeoff between hybridization yield of the intended target and the discrimination factor between intended and SNP targets:

1. Equilibrium Hybridization Yield X_{∞} at ΔG°_{intended}=0.

The equilibrium constant for the A+B⇄D+E reaction is

$$Keq = \frac{[ D ] [ E ]}{[ A ] [ B ]} = \frac{{[ D ]}^{2}}{[ A ] [ B ]} ,$$

where A is the target, B is the probe, D is the fluorescent product, and E is the dark product. In a detection reaction, there are no product [D] and [E] initially; therefore any production of D must necessarily be accompanied by production of E, and the concentrations of the two products are always equal.

n is defined as the stoichiometric ratio between the initial concentrations of A and B

$$( n = \frac{{[ A ]}_{0}}{{[ B ]}_{0}} ) .$$

The hybridization yield x is calculated based off the limiting species of target and probe

$$( x = \frac{[ D ]}{min \{ {[ A ]}_{0} , {[ B ]}_{0} \}} ) ,$$

and varies with time. Here, derivation is shown for the case where n≧1, but similar results follow for n<1. With some simplification, the equilibrium hybridization yield X_{∞} can be expressed as a function of stoichiometry n and equilibrium constant K_{eq}:

$$\begin{matrix}{K}_{eq} = \frac{{x}_{∞}^{2}}{( 1 - {x}_{∞} ) ( n - {x}_{∞} )} & ( Eq . 3 ) \\ {x}_{∞} = \frac{{K}_{eq} + {K}_{eq} \cdot n - \sqrt{{K}_{eq}} \sqrt{{{K}_{eq} ( n - 1 )}^{2} + 4 n}}{2 ( {K}_{eq} - 1 )} & ( Eq . 4 )\end{matrix}$$

Except in the case of K_{eq}=1, in which case

$${x}_{∞} = \frac{n}{n + 1} .$$

2. Discrimination Factor Q at ΔG°_{intended}=0.

The discrimination factor Q is defined as:

$$Q = \frac{{x}_{∞} ( intended )}{{x}_{∞} ( SNP )}$$

With ΔG°_{intended}=0, the numerator becomes

$$\frac{n}{n + 1} .$$

The denominator can be calculated based on substituting ΔG°_{SNP}=ΔΔG°_{ds} into equation (4). The value of ΔΔG°_{ds} depends on the identity and local neighborhood of the SNP, and generally varies between +6 and +10 kcal/mol at room temperature. In this example, the exact value of x_{∞} (SNP) does not matter; it is merely desired to establish a lower bound on Q.

At very high values of n, the system may lose specificity because the overwhelming amount of SNP target can produce equilibrium yield X_{∞}>0.5. This is discussed in detail below. Here n is limited to lower values in which X_{∞}<0.5 (e.g. n<1000). In this case, the value of Q can be bound as follows:

$${K}_{eq} = \frac{{x}_{∞}^{2}}{( 1 - {x}_{∞} ) ( n - {x}_{∞} )} < \frac{{x}_{∞}^{2}}{0.5 ( n - 0.5 )}$$

$$\frac{2 n - 1}{4} {e}^{- Δ Δ {G}_{ds}^{∘} / RT} < {x}_{∞}^{2}$$

$$\sqrt{\frac{2 n - 1}{4}} {e}^{- Δ Δ {G}_{ds}^{∘} / RT} < {x}_{∞} ( SNP )$$

$$Q = \frac{{x}_{∞} ( intended )}{{x}_{∞} ( SNP )} > \frac{\frac{n}{n + 1}}{\sqrt{\frac{2 n - 1}{4}} {e}^{- Δ Δ {G}_{ds}^{∘} / 2 RT}} = \frac{2 n}{( n + 1 ) \sqrt{2 n - 1}} {e}^{ΔΔ {G}_{ds}^{∘} / 2 RT} \geq \frac{1}{\sqrt{2 n - 1}} {e}^{ΔΔ {G}_{ds}^{∘} / 2 RT}$$

TABLE 1
Effects of stoichiometry n on hybridization yield of intended and
SNP targets, and corresponding discrimination factor Q:

| Stoichiometry n | x_{∞} (intended, ΔG^{o} = 0.00 kcal/mol) | x_{∞} (SNP, ΔG^{o} = +6.00 kcal/mol) | $Q = \frac{{x}_{∞} ( intended )}{{x}_{∞} ( SNP )}$ |
|---|---|---|---|
| 1 | 0.500 | 0.0063 | 79.4 |
| 10 | 0.909 | 0.0199 | 45.7 |
| 100 | 0.990 | 0.0615 | 16.1 |
| 1000 | 0.999 | 0.182 | 5.50 |

The value of Q increases monotonically as ΔG°_{intended} increases. At the limit of ΔG°_{intended}→+∞,

$${K}_{eq} = \frac{{x}_{∞}^{2}}{( 1 - {x}_{∞} ) ( n - {x}_{∞} )}$$

$${e}^{- Δ G° / RT} = {x}_{∞}^{2}$$

$${x}_{∞} ( intended ) = {e}^{- Δ {G}_{intended}^{∘} / 2 RT}$$

$${x}_{∞} ( SNP ) = {e}^{- Δ {G}_{SNP}^{∘} / 2 RT} = {e}^{- ΔΔ {G}_{ds}^{∘} / 2 RT} \cdot {e}^{- Δ {G}_{intended}^{∘} / 2 RT}$$

$${Q}_{ma x} = \frac{{x}_{∞} ( intended )}{{x}_{∞} ( SNP )} = {e}^{ΔΔ {G}_{ds}^{∘} / 2 RT}$$

Thus, at ΔG°_{intended}=0 and reasonably small n<1000, the discrimination factor is within a small constant factor

$$\frac{1}{\sqrt{2 n - 1}}$$

of optimal.

3. Representative Equilibrium Hybridization Yields and Discrimination Factors

The solid lines shown in FIG. 17 plots the analytic dependence of x_{∞} on stoichiometry n shown in Equation (4), assuming the following best-fit ΔG° values:

| Target | ΔG ° (kca/mol) |
|---|---|
| Intended (black) | −0.2 |
| i8TA (red) | +3.9 |
| d8 (green) | +5.10 |
| m8CG | +6.0 |

Table 1 (above) shows analytic equilibrium hybridization yields for representative values of n and ΔG°. Dividing the equilibrium hybridization yield for ΔG°=0.00 by that for ΔG°=+6.00 kcal/mol, the analytic discrimination factor Q is obtained. Although discrimination factor is highest at n=1, the SNP can still be effectively distinguished at 1000× excess of target over probe. Because analysis is symmetrical with respect to A and B, stoichiometries n<1 will yield the same discrimination factor as stoichiometry

$${n}^{′} = \frac{1}{n} .$$

### Mathematical Relation Between R and Q.

The concentration equivalence R is an alternative measure of specificity, and denotes the ratio of the quantity of SNP versus intended target needed to yield the same level of fluorescence (50% of maximum here): Conceptually, an R-fold excess of the SNP target yields a false positive, so R determines the specificity of a diagnostic assay based on this technology.

Hybridization Probes

For typical (A+B→D) hybridization probes, R≈Q. First, the derivation for Q is shown in the case of a known n:

$${K}_{eq} = \frac{[ D ]}{[ A ] [ B ]} = \frac{{x}_{∞}}{( n - {x}_{∞} ) {( 1 - {x}_{∞} ) [ B ]}_{0}}$$

$${x}_{∞} = \frac{n + 1}{2} + \frac{1}{2 {{K}_{eq} [ B ]}_{0}} - \frac{\sqrt{\begin{matrix}{{K}_{eq}^{2} [ B ]}_{0}^{2} {n}^{2} + {{K}_{eq}^{2} [ B ]}_{0}^{2} + 1 + \\ 2 {{K}_{eq}^{2} [ B ]}_{0}^{2} n + 2 {{K}_{eq} [ B ]}_{0} - {{K}_{eq} [ B ]}_{0} n\end{matrix}}}{2 {{K}_{eq} [ B ]}_{0}}$$

The last expression of x_{∞} is rather complicated, but if it is assumed that x_{∞}<<0.5 (and thus in the specific regime of the reaction), the solution for simplifies to:

x_{∞}≈K_{eq}[B]₀n=e^{-ΔG°/RT}[B]₀n

The discrimination factor Q is calculated as:

$$\begin{matrix}Q = \frac{{x}_{∞} ( intended )}{{x}_{∞} ( SNP )} \\ \approx \frac{{{e}^{- Δ {G}_{intended}^{°} / RT} [ B ]}_{0} n}{{{e}^{- Δ {G}_{SNP}^{°} / RT} [ B ]}_{0} n} \\ = {e}^{- Δ {G}_{intended}^{°} / RT + Δ {G}_{SNP}^{°} / RT}\end{matrix}$$

Wherein ΔΔG°_{SS}=ΔG°_{SNP}−ΔG°_{intended}, and the above simplifies to:

Q=e^{-ΔG°}^{ₛₛ}^{/RT}

Next, R is derived based on x_{∞}=0.5:

$${e}^{- Δ G ° / RT } = {K}_{eq} = \frac{[ D ]}{[ A ] [ B ]} = {\frac{{x}_{∞}}{( n - {x}_{∞} ) {( 1 - {x}_{∞} ) [ B ]}_{0}} \\ [ B ]}_{0} ( n - 0.5 ) = {e}^{Δ G ° / RT }$$

$$n = \frac{{[ B ]}_{0} + 2 {e}^{Δ G ° / RT}}{{2 [ B ]}_{0}}$$

From this expression, R is solved:

$$R = \frac{{n}_{SNP}}{{n}_{intended}}$$

$$R = \frac{{[ B ]}_{0} + 2 {e}^{Δ {G}_{SNP}^{°} / RT}}{{[ B ]}_{0} + 2 {e}^{Δ {G}_{intended}^{°} / RT}}$$

Further, ΔΔG°ₛₛ=ΔG°_{SNP}−ΔG°_{intended}, is used and the above simplifies to:

$$R = \frac{{[ B ]}_{0} + 2 {e}^{Δ {G}_{ss}^{°} / RT} {e}^{Δ {G}_{intended}^{°} / RT}}{{[ B ]}_{0} + 2 {e}^{Δ {G}_{intended}^{°} / RT}}$$

When the intended hybridization is too favorable

$$( {e}^{Δ {G}_{intended}^{°} / RT} << {[ B ]}_{0} ) ,$$

the reaction is not specific, and the concentration equivalence approaches 1. In contrast, when

$$( {e}^{Δ {G}_{intended}^{°} / RT} > {[ B ]}_{0} ) ,$$

concentration equivalence can be approximated as:

$$R \approx {e}^{Δ {G}_{ss}^{°} / RT} ) .$$

Thus, R≈Q for the A+B→D hybridization probe reaction in the specific regime of the probe.

Double-Stranded Toehold Exchange Probes

For the A+B⇄D+E reaction with ΔΔG°_{ds}=ΔG°_{SNP}−ΔG°_{intended}, similar logic as the above leads to:

$${x}_{∞} \approx \sqrt{{ne}^{{ }^{- Δ {G}^{°} / RT}}}$$

$$Q = {e}^{Δ {G}_{ds}^{°} / 2 RT}$$

Similarly for R:

n=1+e^{ΔG°/RT}

$$R \approx {e}^{Δ {G}_{ds}^{°} / RT}$$

Thus, for the double-stranded probe, R≈Q² in the specific regime where hybridization yields are below 0.5. Experimentally, the median discrimination factor Q that is observed is 43; consequently, up to an R=Q²≈1800-fold excess of an SNP target will be tolerated before yielded the same fluorescence signal as 1× of the intended target.

Note that ΔG°_{ds}≈2ΔG°ₛₛ, since the double-stranded probe results in 2 mismatch bubbles per base pair change in the target, whereas the conventional hybridization probes results in 1 mismatch bubble per base change. Thus, the observed discrimination factor Q will be roughly the same for the double-stranded probe as under the conventional hybridization probe under optimal conditions, but the concentration equivalence R will be roughly quadratically larger.

### Example 4

### Conditionally Fluorescent Molecular Probes for Detecting Single Base Changes in Double-Stranded DNA

As shown in the Examples below, a class of conditionally fluorescent molecular probes are presented that effectively discriminate single base changes in primarily double-stranded DNA (dsDNA), work robustly for a wide range of conditions and identify single base changes within long stretches of DNA. This approach relies on a mechanism described herein as ‘double-stranded toehold exchange’ and uses a rationally designed double-stranded probe with forked single-stranded overhangs (FIG. 14a). The probes were first demonstrated on a set of synthetic dsDNA targets. The reaction of the probe with any SNP target (a molecule that differs from the intended target by a single base pair) exhibited negligibly low yield. Experimentally, up to a 12,000-fold excess of a target was needed to achieve the same signal as a stoichiometric amount of the intended target, comparable to the best enzyme-based methods. Subsequently, this approach was extended to the detection of point mutations in the E. Coli rpoB gene. The probes described herein correctly identified point mutations responsible for resistance to the antibiotic rifampicin (Severinov et al. 1993; Telenti et al. 1993).

These studies focus on the problem of constructing high-specificity probes for SNP detection, but the double-stranded toehold exchange mechanism may also be used for a variety of other applications, such as the modular construction of complex biomolecular circuits for the logical and temporal control of DNA (Zhang & Seelig 2011a; Seelig et al. 2006; Zhang et al. 2007; Soloveichik et al. 2010; Zhang & Winfree 2010; Qian & Winfree 2011) and other molecules (Nandagopal & Elowitz 2011; Purnick & Weiss 2009; Bunka et al. 2010).

Methods

Probe Design.

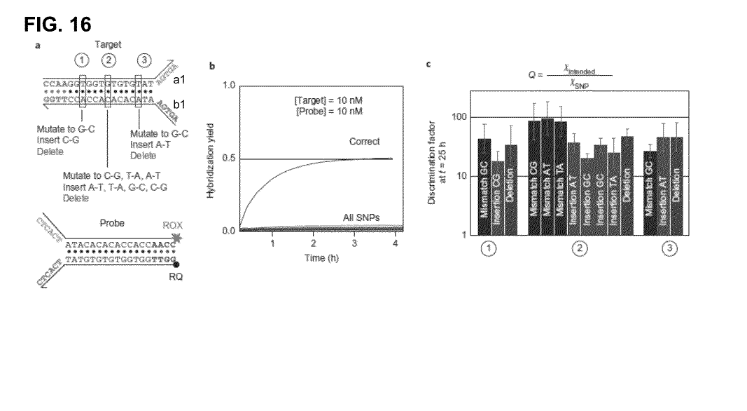

Pairs of forked toeholds were designed with self-similar sequences to avoid secondary structure (for example, for targets shown in FIG. 16 the orange (a1)) toehold is 5′-AGTGA-3′ and the purple (b1) toehold is 3′-AGTGA-5′); this is true for both initiation toeholds and dissociation toeholds. This allows the accurate prediction of probe-binding thermodynamics and kinetics. DNA oligonucleotides. All DNA oligonucleotides were purchased from Integrated DNA Technology (IDT). Fluorophore- and quencher-labelled oligonucleotides were HPLC purified, non-labelled oligonucleotides (shown in FIG. 19) were purchased as Ultramers, primers for PCR were unpurified and all remaining oligonucleotides were purified by polyacrylamide gel electrophoresis. Individual DNA oligonucleotides were resuspended to 100 mM and stored in elution buffer (10 mM TrisHCl, pH 8.5; Qiagen).

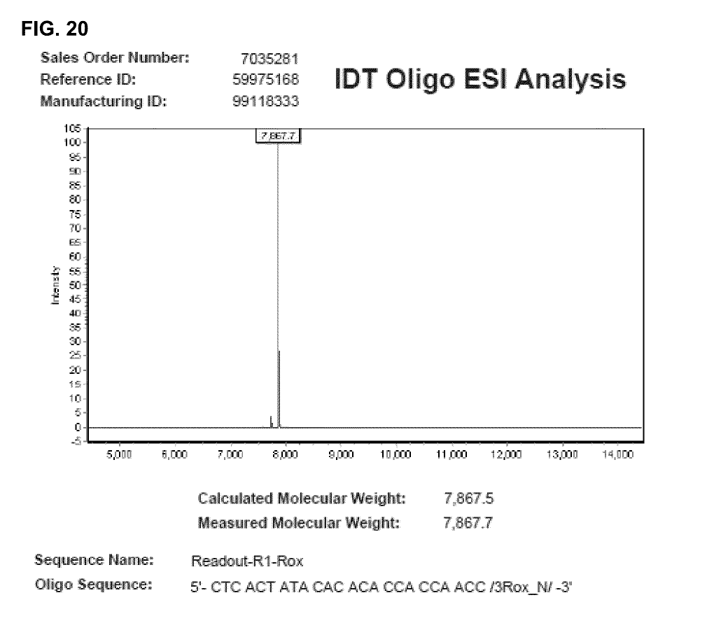

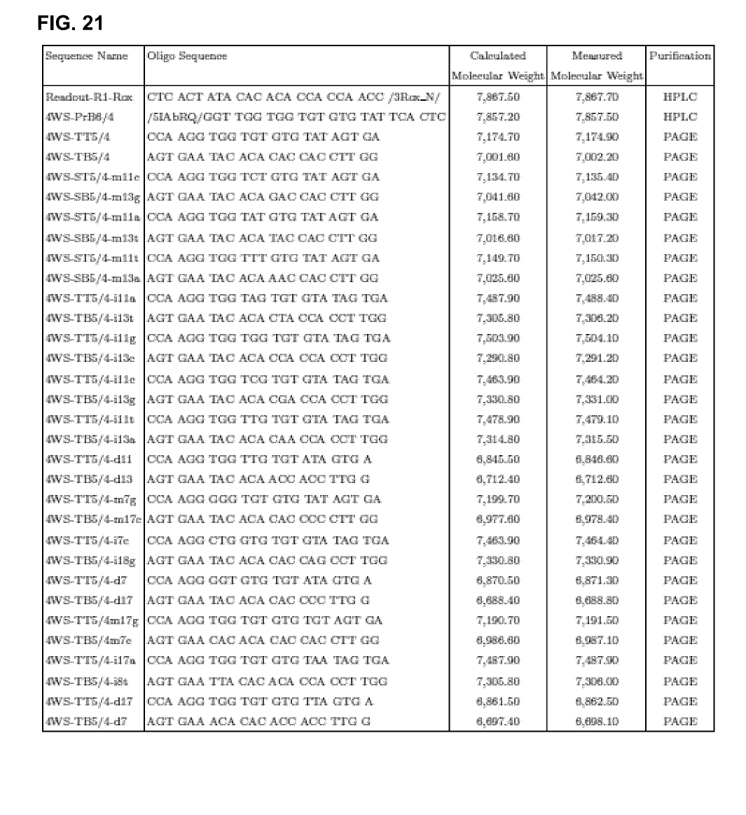

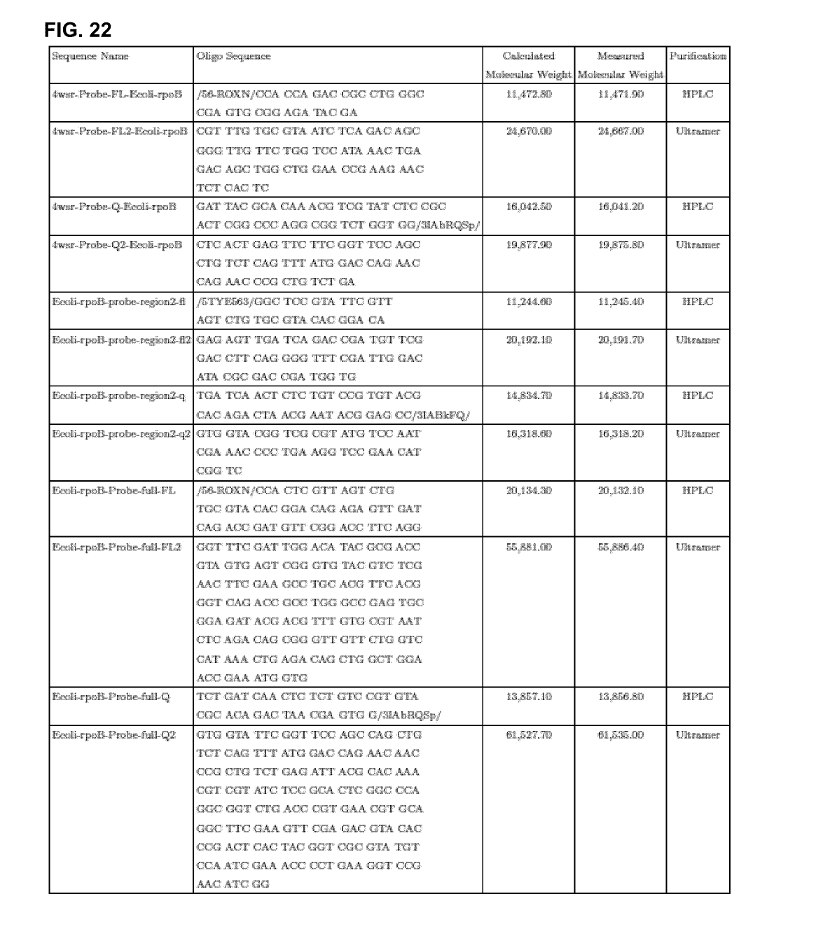

IDT provided electrospray ionization (ESI) mass spectrometry data sheets as the quality control; FIG. 20 shows one representative ESI spectrum and FIGS. 21 and 22 shows a summary of all ESI results (all 47 oligonucleotides showed over 75% purity and 45 oligonucleotides were over 90% pure).

Probe Preparation.

Probe molecules consisted of either two distinct strands (FIGS. 16-18) or four distinct strands (FIG. 19). The strands were mixed stoichiometrically and then thermally annealed (Biorad T100), and cooled uniformly from 98° C. to 25° C. over the course of 73 minutes. To ensure stoichiometry, two-stranded probe molecules were then gel purified using a 10% polyacrylamide gel; four-stranded probe molecules were not purified.

Gel solutions were prepared from 40% 19:1 acrylamide:bisacrylamide stock (J. T. Baker Analytical) in 1× Tris-acetate-EDTA buffer/Mg2+ solution, and cast between 20 cm by 20 cm glass plates with 1.5 mm spacers. Samples were loaded with 80% glycerol to achieve 10% glycerol concentration by volume. The gel was run at room temperature using a Hoefer SE600 chamber at 140 V for four hours. Gel bands were visualized using Entela UL3101 ultraviolet light with a fluorescent backplate (Whatman UV254 Polyester 4410222), and then cut out and eluted into 1 ml buffer.

Target Preparation.

The target molecules shown in FIGS. 16-18 were prepared similarly to the two-stranded probes, via annealing and purification as described above. The targets derived from E. Coli shown FIG. 19 were constructed in a multistep process. Competent cells (10 ul) were cultured in 5 ml Luria Broth (LB) at 37° C. for 48 hours, and then split into 1 ml aliquots. Each aliquot was centrifuged for ten minutes, after which supernatant was decanted. Precipitate was resuspended in 200 μl LB and plated on a LB plate with either 0 μg ml⁻¹ WT or 300 μg ml⁻¹ rifampicin (Sigma R3501). Colonies were allowed to form overnight at 37° C.

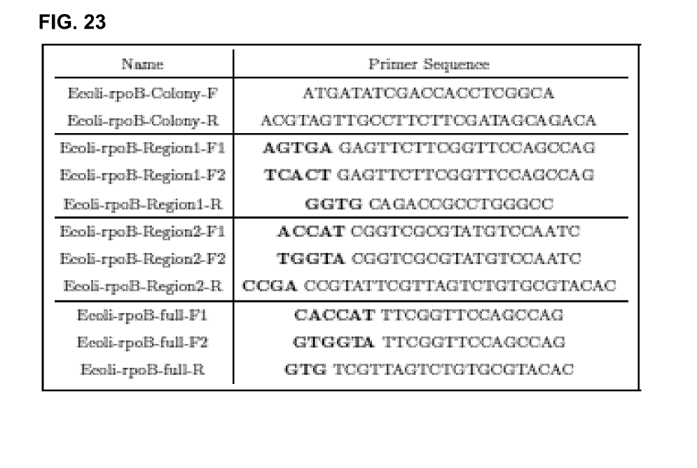

Ten rifampicin-resistant colonies were picked from the plates, and each was amplified with colony PCR (13 minutes at 98° C., followed by 40 cycles of 98° C. (15 s), 50° C. (˜20 s) and 72° C. (˜40 s), followed by five minutes at 72° C. (Biorad T100)) with 500 nM of each primer (primers shown in FIG. 23). Subsequently, colony PCR-amplification products were used as templates for unbalanced PCR (with 500 nM of one primer and 5 nM of the other 40 cycles). Both primers had a designed toehold at the 5′ end that would serve as initiation or dissociation toehold experiments. The two strands of the target were constructed using unbalanced PCR separately, and annealed afterwards.

Time-Course Fluorescence Studies.

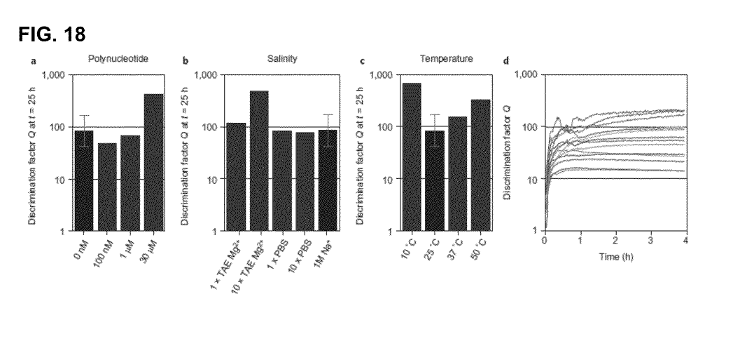

Kinetic fluorescence measurements were performed using a Horiba FluoroMax 3 spectrofluorometer and Hellma Semi-Micro 114F spectrofluorometer cuvettes. Probes that targeted the 562-576 codon region of the rpoB gene (FIG. 19) used the TYE563 fluorophore (excitation 549 nm, emission 563 nm). For all other experiments, probes used the ROX fluorophore (excitation 584 nm, emission 603 nm). Slit sizes were set at 5 nm for all monochromators. An external temperature bath maintained a designated reaction temperature (25±1° C., unless stated explicitly in FIG. 18).

A four-sample changer was used so that time-based fluorescence experiments were performed in groups of four. For the simultaneous detection experiments shown in FIG. 19, each data point represents the integrated fluorescence over ten seconds per two minutes of reaction. For all other experiments, each data point represents the integrated fluorescence over ten seconds per minute of reaction.

Fluorescence Normalization and Hybridization Yield Inference.

All fluorescence values were normalized and converted into hybridization yields x via the formula x=(F−F_{b})/(Fₛ−F_{b}), where F is the observed fluorescence, F_{b} is the background fluorescence observed with the addition of buffer only and Fₛ is the saturated fluorescence observed after the addition of a 40-fold excess of correctly matched target (see FIG. 15 for details).

Results

To test whether the fluorescent probe based on double-stranded toehold exchange functions as intended, an arbitrary dsDNA sequence of 14 base pairs was designed. The initiation and dissociation toeholds (see Methods for sequence design) were then appended to the ends, which resulted in an intended target molecule with 18 base pairs. To facilitate experimentation on the effects of the reaction ΔG°, the probe was designed to each possess six nucleotides of initiation toehold, but the effective toehold is determined by the shorter of the toeholds for the probe and the target, and the latter was designed to be five nucleotides for the experiments shown here in the Example below. Fourteen SNP target molecules were designed that each differed from the intended target by one base pair (FIG. 16a). These base changes were distributed at three different positions along the testing region, and included insertions, deletions and replacements.

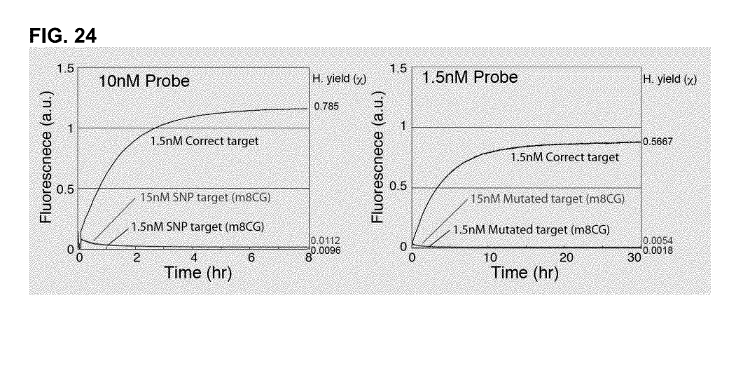

FIG. 16b shows the inferred hybridization yield

$$x = \frac{[ D ]}{{[ B ]}_{0}} ,$$

where D is the fluorescent product and [B]₀ is the initial concentration of probe (for all experiments, [A]≧[B]). (See Methods and FIG. 15 for details on the derivation of x from raw fluorescence values.) The discrimination factor, Q=X_{intended}/x_{SNP}, quantifies the single-base specificity of the probe as the ratio of the hybridization yields (fluorescence) generated by equal concentrations of the intended and SNP targets; Q at t=25 h is plotted in FIG. 16c, and ranges from 17 to 99, with a median of 43. Operation of the probe is robust to the concentration of the probe; at lower probe concentrations, kinetics are slower, but discrimination at equilibrium is preserved (FIG. 24).

For spurious targets that differ from the intended target by more than one base pair, analysis predicts that the discrimination factor will be roughly exponential in the number of base-pair changes (for example, a spurious target with two base-pair changes would yield a discrimination factor of roughly Q≈43²≈1,800). However, the sensitivity of the equipment precludes the accurate measurement of hybridization yields lower than about 0.002; consequently, spurious targets that differ by two or more base pairs were not tested.

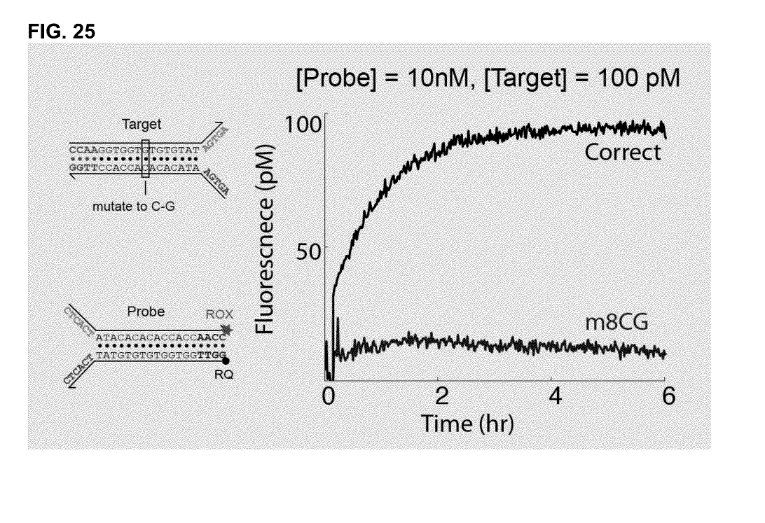

It is important that, as for molecular beacons and other nucleic acid hybridization probes, the double-stranded probe does not in itself employ molecular or fluorescence amplification. For the E. Coli experiments discussed below, the colony polymerase-chain reaction (PCR) step provided the amplification to generate enough target for fluorescence analysis. Without amplification, the sensitivity of the probes described herein is limited by the sensitivity of the fluorescence readout. For a typical fluorometer, the sensitivity limit is around 100 μM of unquenched fluorophores (FIG. 25).

Concentration Equivalence.

The concentration equivalence (R) denotes the excess of SNP target needed to yield the same level of fluorescence (50% of maximum) as that of the intended target at an equal concentration to that of the probe. An R-fold excess of the SNP target yields a false positive, so R determines the specificity of a diagnostic assay based on this technology. For typical hybridization probes, R≈Q; however, for the double-stranded probes described herein, the value of R is approximately Q2 (see below for mathematical details). The quadratic relation between R and Q occurs because there are two products with concentrations that, to maintain equilibrium, will increase to compensate for an increase in target concentration. For the same value of

$${K}_{eq} = \frac{[ D ] [ E ]}{[ A ] [ B ]} ,$$

both [D] and [E] increase to balance out an increase in [A]. Thus, an increase in the amount of SNP targets only has a square-root effect on the observed fluorescence (rather than a linear effect). Consequently, high stoichiometric ratios of SNP targets have a much smaller effect on the double-stranded probes than on standard hybridization probes.

FIG. 17b shows the response of the probe to various concentrations of the intended target and one particular SNP target, 18TA′. Whereas 10 nM of the intended target results in approximately 50% hybridization yield at equilibrium (c_{∞}=0.5), between 1 and 8 μM of the SNP target is needed to generate the same yield (fluorescence), which indicates that R for ‘i8TA’ is between 100 and 800. Similar experiments were performed for two additional SNP targets, ‘d8’ and ‘m8CG’, and the hybridization yield x of these reactions at t=25 h is plotted against the concentrations of the SNP targets in FIG. 17c. The solid lines in FIG. 17c show the analytic dependence of equilibrium x on the concentrations of the intended and SNP targets using best fit for reaction ΔG° (see below). Listed R values show the horizontal distance between the black curve for the intended target and the colored curves for the SNP targets. The listed Q values are determined from the hybridization yields at 1:1 stoichiometry of target to probe after 25 hours of reaction. Thus, experiments verify that R varies roughly as the square of Q.

Robustness.

Diagnostic assays of DNA samples benefit from solution robustness because biological samples or PCR products can be analyzed directly without separate purification and/or buffer-exchange procedures. The primarily double-stranded nature of the targets and probes confers a high degree of robustness to non-cognate single-stranded nucleic acids in solution. FIG. 18a shows that discrimination is robust in up to 30 μM of a 50 nucleotide polynucleotide-sequence mixture (in which every position has roughly equal probability of being G, C, A or T). In contrast, probes and reactions based on single-stranded oligonucleotides are affected significantly by a 1 μM polynucleotide sequence mixture (Zhang & Winfree 2010).

Similarly, temperature-robust diagnostic assays would be desirable in point-of-care and/or resource-limited settings in which precise temperature-control equipment may not be available. At different salinities or temperatures, the ΔG°≈0 property required for high specificity is preserved because changes to the thermodynamic favorability of base pairing affects both the reactants and products equally; consequently, probes should be highly specific at equilibrium across a wide range of temperatures and salinities. Experimentally, the probes showed high discrimination at equilibrium between the intended target and the SNP target in 1×PBS, 10×PBS, 12.5 mM Mg²⁺ and 125 mM Mg²⁺ (at 25° C., FIG. 18b) and at 10° C., 25° C., 37° C. and 50° C. (in 1 M Na⁺, FIG. 18c).

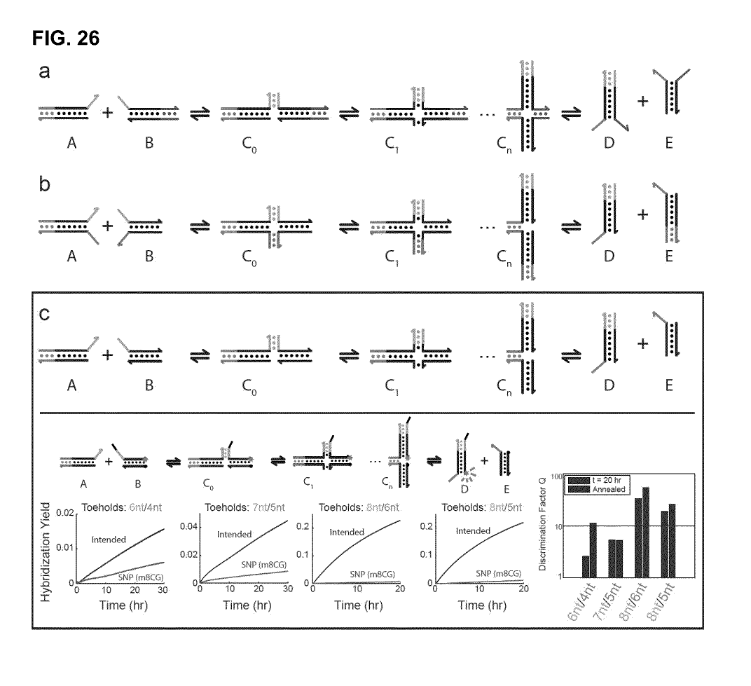

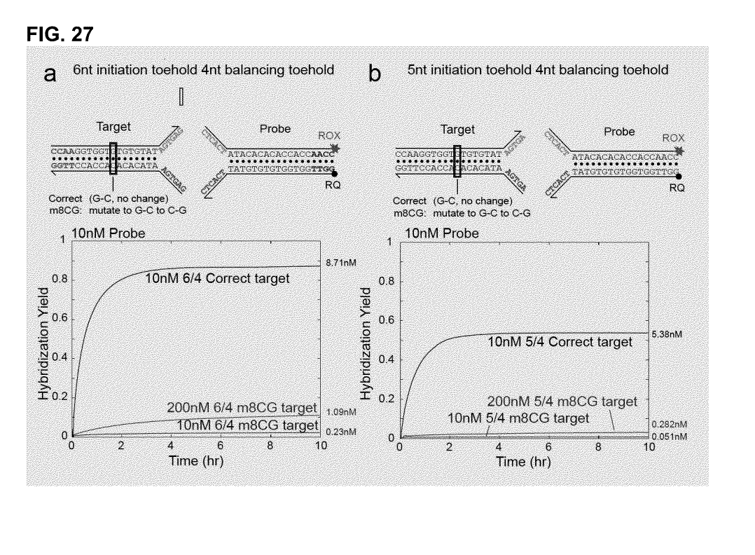

Next, it was determined how quickly the probes could distinguish an SNP target from an intended target. For this, the hybridization yields for the data in FIG. 16b was calculated as a function of time; that is, the fluorescence values for an SNP target was divided by the fluorescence value for the intended target at each time point (FIG. 18d). It was found that, for all SNP targets, Q>10 in less than 20 minutes after the initiation of the reaction; thus a reliable result is obtained long before the detection reaction reaches equilibrium, and this high Q is maintained indefinitely (FIG. 18d). In contrast, other dsDNA probes that utilize four-way branch migration (Panyutin & Hsieh 1993; Biswas et al. 1998; Lishanski 2000; Yang et al. 2003; Liu et al. 2000) do not use the dissociation toeholds and discriminate SNPs using kinetics. Consequently, at equilibrium, both intended and SNP targets are nearly 100% bound to the probe and discrimination is only possible at early time points in the reaction, which increases the possibility of false-positive results (FIG. 26 and FIG. 27).

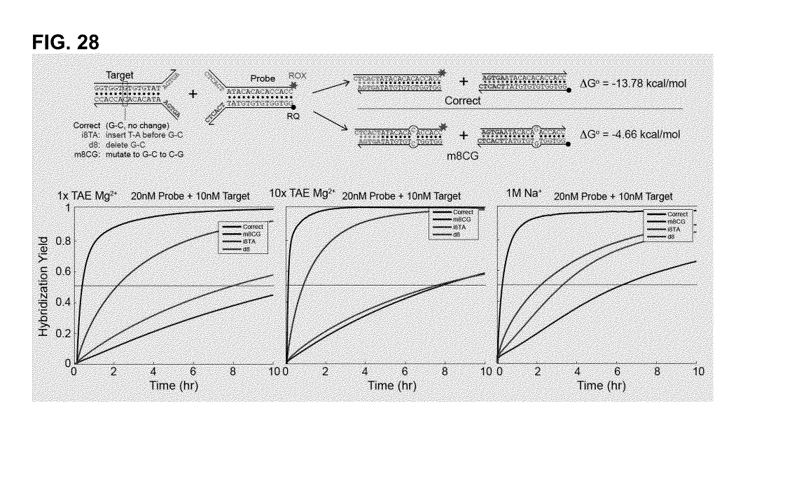

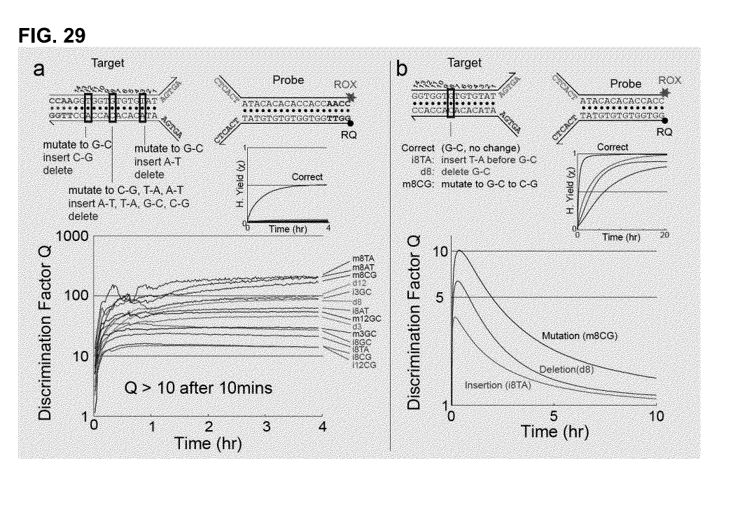

Finally, a number of experiments that compared the SNP discrimination performance of the dsDNA probes were performed to those of molecular beacons (FIG. 28 and FIG. 29). In all cases, dsDNA probes showed significantly higher discrimination factors; this was particularly true for experiments that targeted a subsequence of the E. Coli rpoB gene, as the target had a significant secondary structure. The dsDNA probes were not affected by the secondary structure, and furthermore could reliably distinguish one nucleotide change within 198 nucleotides, whereas molecular beacons were limited to a detection of one nucleotide change within 15 nucleotides.

Biologically Derived Samples.

Single-base mutations in bacterial genomes can confer resistances to antibiotics. For example, the rpoB gene encodes the b subunit of bacterial RNA polymerase; many mutations in rpoB preserve polymerase function but confer rifampicin resistance in E. Coli, Mycobacterium tuberculosis and other bacteria (Severinov et al. 1993; Telenti et al. 1993).

To prevent widespread antibiotic resistance, it is desirable to treat non-rifampicin-resistant infectious bacteria with rifampicin, and resort to less widespread drugs only when necessary. To facilitate such tactical use of antibiotics, fast, accurate and low-cost drug resistance assays are needed.

As a proof-of-concept demonstration that double-stranded toehold-exchange probes can be used for drug-resistance assays, three probes that targeted subsequences of the rpoB gene (FIG. 19a) were designed and tested. The two shorter probes tested nucleotides 1,531-1,599 and 1,684-1,728 (corresponding to codons 511-533 and 562-576). The probes were functionalized with spectrally distinct fluorophores (ROX and Tye563) and operated simultaneously in solution. The longer probe tested the entire 198 base-pair subsequence from 1,531 to 1,728 (codons 511-576). The target DNA was generated from E. Coli colonies via a two-step process: colony PCR was first used to preamplify the rpoB subsequence, and then unbalanced PCR was used to generate each of the two strands that comprise the target (FIG. 19b).

Probes used here were discontinuous and were assembled from four complementary overlapping sequences, rather than just two (FIG. 19b). This design change was necessary because fluorophore- or quencher-functionalized oligonucleotides of more than 50 nucleotides cannot be synthesized efficiently. The experiments revealed that the probe effectively discriminates SNPs despite nicks in the branch-migration region.

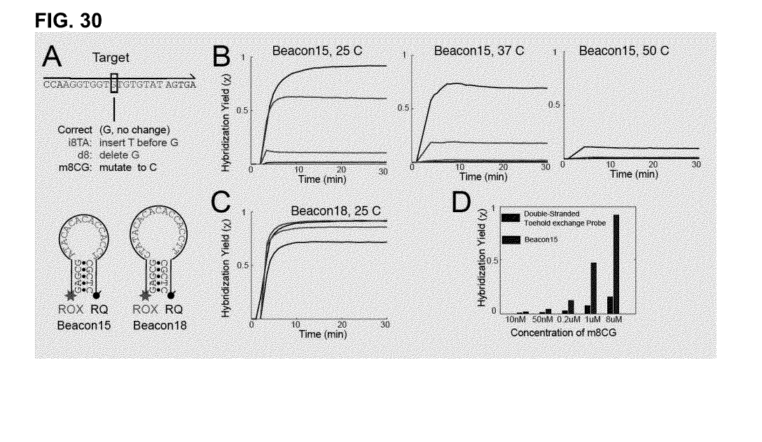

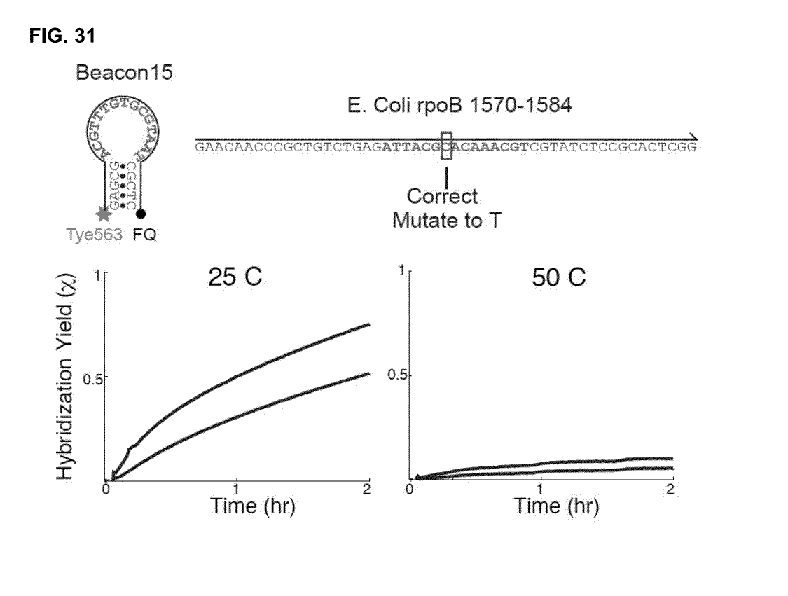

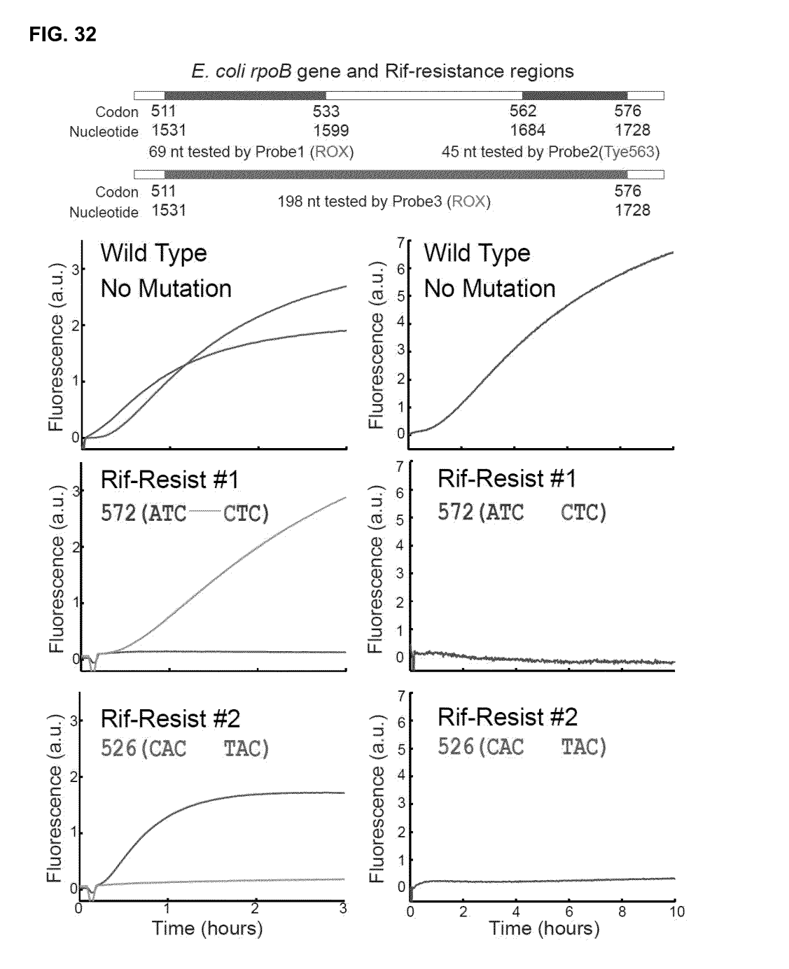

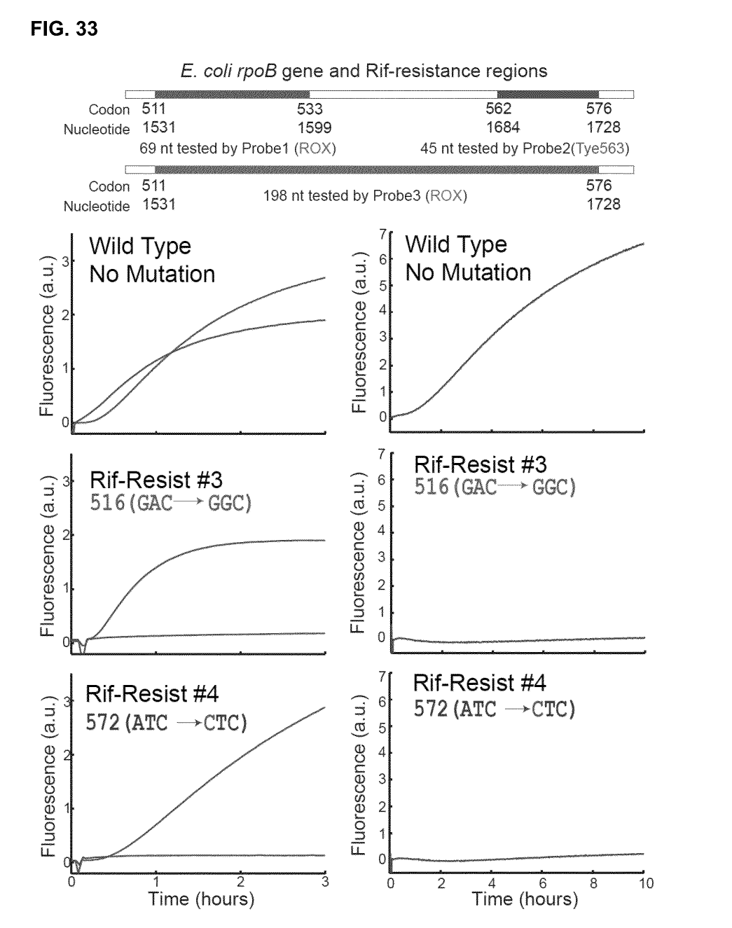

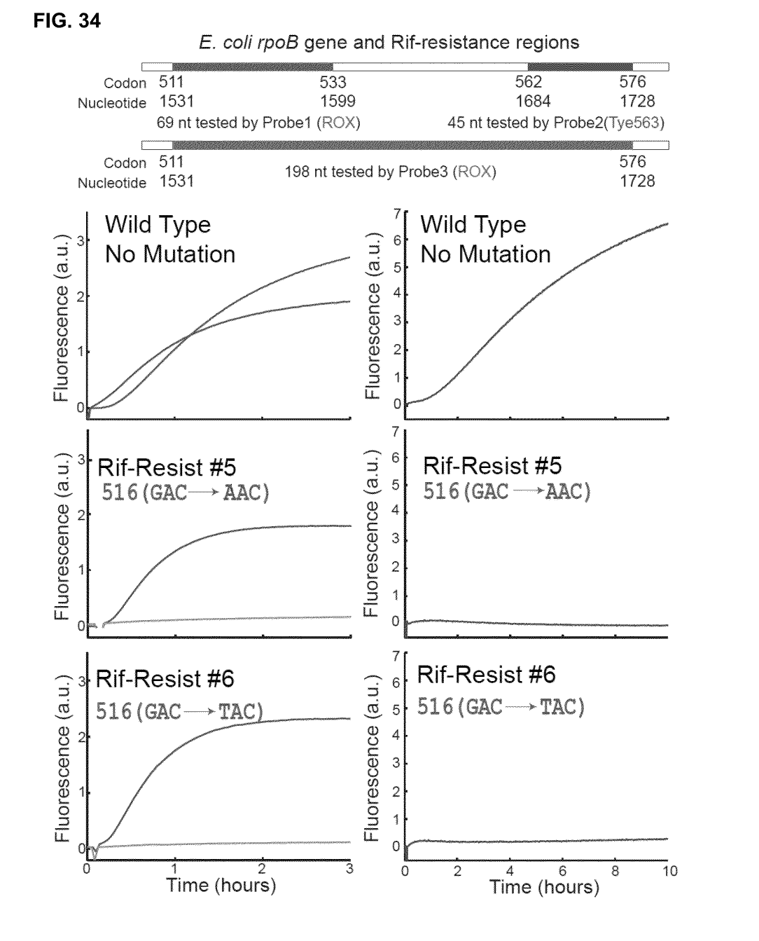

Experimentally, DNA from all ten rifampicin-resistant colonies exhibited mismatch behavior in either the 1,531-1,599 region or the 1,684-1,728 region; in contrast, the wild-type (WT) DNA induced increases in fluorescence for probes that targeted both regions (FIG. 19c; FIGS. 30-34). Sequencing confirmed the presence of one or more mutations in these two regions for all ten colonies.

The probes for the 1,531-1,599 region and those for the 1,684-1,738 region utilized different toehold sequences; the difference in toehold strengths may have contributed to the different kinetics observed for the blue (2) and green (1) traces in FIG. 19c. Kinetics are highly sensitive to toehold thermodynamics (for example, a 0.4 kcal mol⁻¹ change results in a twofold difference in kinetics). However, equilibrium-based probes, such as the double-stranded toehold-exchange probes presented here, are robust to these kinetic differences.

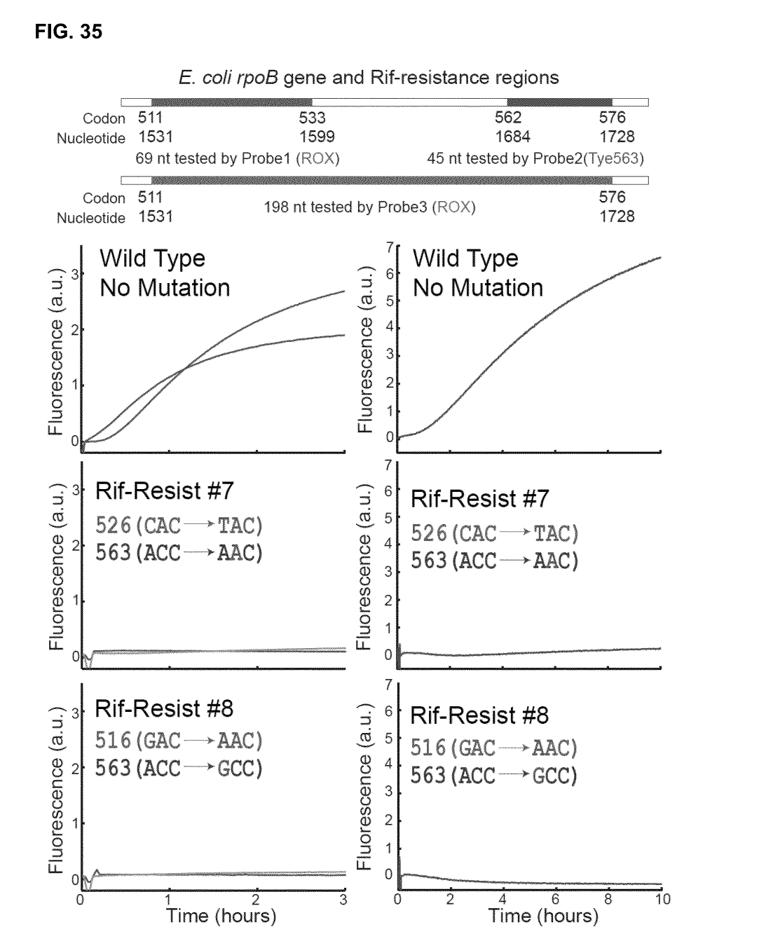

It was also demonstrated that similar probes and targets with only one initiation toehold and one dissociation toehold function to discriminate single base changes reliably, albeit with slower kinetics than probes that use forked toeholds (FIG. 35). Target and probe molecules with only a single overhang are prepared more easily from biological samples, for example using a restriction enzyme or PCR with a modified primer.

Discussion

Using the mechanism of double-stranded toehold exchange, a novel technology for the effective detection of individual base-pair changes in dsDNA was developed. This discrimination method worked reliably for a wide range of mutations at different positions within a duplex, with a median discrimination factor of Q=43. Detection was robust to changes in temperature and salinity, and even to up to 30 mM of a mixture of non-cognate single-stranded DNA (which represents a 3,000-fold excess over probe and target concentrations). Depending on the type of base change, a 260- to 12,000-fold excess of the SNP target was tolerated before the signal caused by the mutated target became comparable to that of a correct target.

The high specificity of the probes derives from a combination of two factors:

(1) The probes were rationally designed to react with the intended target with ΔG°≈0, at which slight thermodynamic changes caused by a single-base mismatch have a disproportionately large effect on the hybridization yield. Specificity is reduced when ΔG°<0, for example when initiation toeholds are significantly longer than dissociation toeholds (FIG. 36).

(2) The assay described herein has the advantage that a base-pair change in the target leads to the formation of two mismatch bubbles in the reaction products. Assays that probe single-stranded targets yield only one mismatch bubble per base change, and the smaller ΔG° change between correct and SNP targets cannot be distinguished as easily on the basis of thermodynamics.

It was further shown that correct and mutated targets can be clearly distinguished early during the approach to equilibrium. Under conditions in which reaction equilibration occurs on the time scale of hours, all mutated targets were identified within the first 20 minutes of the reaction, and fluorescence discrimination was maintained for the remainder of the 25 hours in which the reactions were observed.

The ability to identify single point mutations is critical for diagnosing antibiotic resistance in tuberculosis and other diseases (McNerney & Daley 2011; Niemz et al. 2011; Piatek et al. 1998; Boehme et al. 2010), because most drug resistances can be traced to individual point mutations in narrowly defined regions within a few genes (see Bang et al. 2011). The probes described herein can be used to screen extended genetic regions (that potentially contain multiple SNPs in different positions) and can be multiplexed to screen mutations that occur in different genes, which makes this a promising technology for developing rapid and reliable infectious-disease diagnostics. To test this, a method for the detection of mutations that confer rifampicin resistance to E. Coli was used. Probes were constructed to test two highly variable regions of the rpoB gene and also a 198 base-pair domain that encompasses both regions. DNA from WT E. Coli and ten different resistant colonies were reacted with probes, and sequencing confirmed that probes correctly detected the presence of single base-pair changes in all cases. Furthermore, multiplexing was demonstrated—two probes labelled with different fluorophores functioned simultaneously in the same detection reaction. This could allow the use of an internal control that probes a conserved sequence to compensate for sample-to-sample variability.

These experiments on biologically derived target DNA utilized discontinuous probes that contain non-overlapping nicks. With this approach, the ‘read length’ of molecular probes was increased significantly. Traditionally, read length is limited to approximately 50 nucleotides or fewer, because of synthesis limitations and also reduced specificity of long oligonucleotide hybridization. Here, the ability to detect a single base change in a continuous region of DNA that is 198 base-pairs long was demonstrated, and it is envisioned that this technology can be extended to enable SNP detection in significantly longer DNA, potentially even probing lengths necessary to solve the haplotype phasing problem (Browning & Browning 2011). Furthermore, by using the discontinuous dsDNA probes, a highly accurate ‘sequence comparison’ mechanism was established that may function as a complement to next-generation sequencing technologies (current sequencing reads are limited to less than 500 nucleotides).

The probes may also be useful for the detection of single-stranded targets that are first converted into dsDNA with forked toeholds using PCR-based methods. Single-stranded nucleic acids often have considerable secondary structure at or near room temperature. Such self-interactions within a target or a single-stranded probe can interfere with the detection reaction, both at a kinetic level (Seelig et al. 2006; Gao et al. 2006) and at an equilibrium thermodynamics level (Zhang & Winfree 2010; Zhang et al. 2005). The double-stranded nature of the probes and target discourages undesirable pathways that lead to either kinetic traps or spurious interactions. Such a procedure would not necessarily increase the complexity of the detection process because most existing nucleic acid detection technologies require a PCR-based preamplification step.

### Example 5

### Multi-Input DNA Logic Circuits for Diagnostic Applications

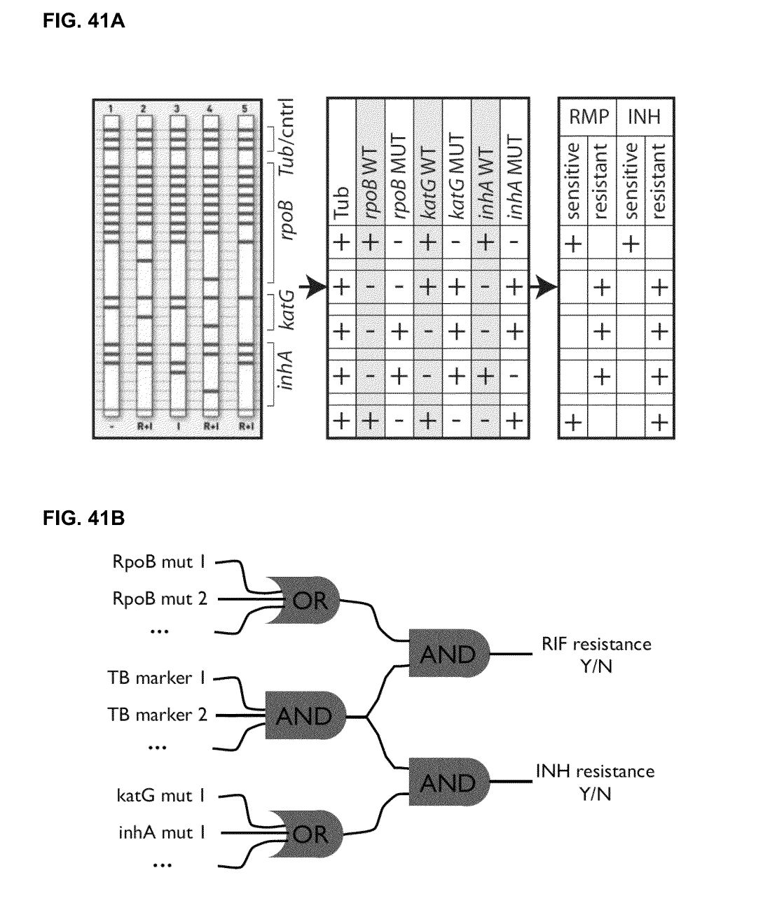

Several amplifier modules may be integrated into a molecular circuit for multi-input analysis. For example, molecular markers associated with the M. tuberculosis complex and with resistance of tuberculosis (TB) to common antibiotics may be detected and analyzed using multi-input DNA logic circuits as described herein. The design may be based on established molecular markers and sequences used in line probe assays (e.g., Hain Genotype MTBDR+, INNO-LiPA TB). However, the complexity of the readout and thus of the diagnostic challenge can be drastically reduced using molecular logic. Instead of reading out each marker independently as is currently done in line probe assays (FIG. 41A, left and center panel), a DNA circuit may be used to analyze the information encoded in these markers and provide a signal corresponding to a diagnostic outcome of interest (FIG. 41B). In these experiments, synthetic single stranded DNA and RNA sequences may be used as inputs. After the experiment is optimized, actual TB samples may be tested using this method. The experiment performed in this Example may be performed in solution phase.

The ability to identify single point mutations is important for diagnosing antibiotic resistance in TB. TB is classified as multi-drug resistant (MDR) if it is resistant to the first-line antibiotics rifampicin (RIF) and isonizid (INH). Extensively drug resistant (XDR) TB is further resistant to second-line antibiotics such as kanamycin. Most of these drug resistances can be traced to individual point mutations in narrowly defined regions within a few genes. For example, mutations in an 81 base pair region in the rpoB gene are responsible for 95% of the observed cases of resistance to RIF. A majority (50-95%) of INH resistant strands carry a mutation in codon 315 of the katG gene and 20-35% carry mutations in the inhA promoter region. Less common mutations responsible for the remaining percentages are also mostly well characterized. This system provides an ideal test case for nucleic acid circuitry because diagnosis of resistance to either antibiotic requires analysis of multiple point mutations in parallel.

A logic circuit diagram for an example diagnostic circuit is shown in FIG. 41B. An amplifier module, as described in Example 1, may be used for detection of each specific mutation. Multiple different mutations that can independently lead to resistance to the same antibiotic are connected with each other in a logical OR clause. Amplifier modules are also used to detect markers associated with the M. tuberculosis complex. Detection of multiple markers can reduce false positive detection rates if the markers are connected in a logical AND. To further simplify the readout, the output signal from the OR operation that tests for resistance may be combined with the result of the TB detection module. Implementation of logical AND and OR gates, using recently demonstrated seesaw gates and thresholds, were used for an experimental implementation of this circuit (Qian et al., 2011).

An important feature of the systems described above is the modularity and scalability of circuits. Additional analytes may be added to an existing circuit and the logic may be easily changed to reflect a different diagnostic need. For example, in the circuit of FIG. 41B it may be desirable to have an independent readout for the TB markers and not use the second layer of logic. In the longer term, the circuit may be extended to include additional markers associated with extensively drug resistant TB. For biosensing applications, DNA circuits should ideally be able to operate in blood serum and similarly complex environments and thus require sample pre-preparation. Very recently, a first example of a DNA strand displacement reaction in serum was demonstrated (Graugnard et al., 2011). Furthermore, there are several examples of DNA circuitry operating in total RNA (Seelig et al., 2006), cell extract (Zhang et al., 2007) and similar complex biochemical environments. The diagnostic circuit described above may be developed using relatively simple reaction environments and may be later tested directly in serum.

### Example 6

### Development of DNA Logic Circuits for Paper-Based Reactions and Visual Readout

In Examples 1 and 2, DNA circuit reactions were carried out in tube-based reactions by manual addition of reagents and were analyzed using a fluorescence detection instrument. In this Example, DNA logic circuits may be adapted to be compatible with paper devices appropriate for point-of-use applications. For detection, DNA circuit outputs may be labeled with gold nanoparticles for visual detection and tested using outputs from tube-based reactions; hybridization kinetics may be measured to determine times for nearly complete capture of the output DNA. For the reaction component, DNA logic circuits as fluid-phase reactions in a paper matrix may be tested. Logic components may also be immobilized onto paper for solid-phase logic reactions. Reaction kinetics may be measured by comparison to tube-based reactions. The DNA logic circuits may be designed with visible detection starting with simple logic circuits and progressing to more sophisticated circuits. For example, the TB assay from Example 2 may be used.

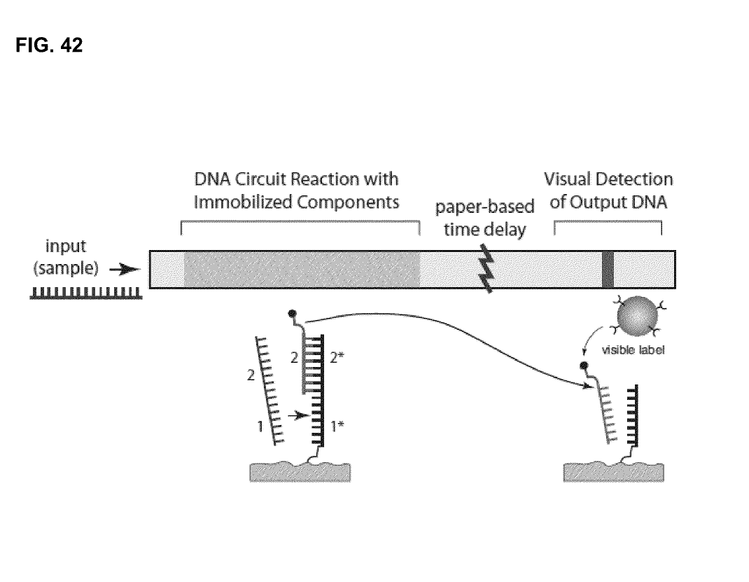

FIG. 42 shows a schematic of a device that motivates the fundamental work in this Example. The basic parts of the strip are a reaction zone and a detection zone for capture and visual detection of labeled DNA circuit outputs. In the reaction zone, the DNA circuit chemistry may be reacted in the fluid-phase or by immobilizing DNA logic components with pre-bound output DNA (this version is shown for the simplest one-input-one-output circuit, FIG. 42). The released output DNA can be captured at the detection line by hybridization to immobilized complementary probes. The reaction and detection zones can be separated by a paper-based time delay (see dissolvable delays discussed below) that can be programmed to match the reaction kinetics for automation of the two-step reaction. The two stages (detection and reaction) may be tested separately at first, and then demonstrated as an integrated system.

First, DNA may be captured and detected using visible labels. In the tube-based reactions of Examples 1 and 2, fluorophores or quenchers were conjugated to the output DNA; here, output DNA may include tags (e.g., FITC, Dig) that can be bound by gold nanoparticles (e.g., anti-FITC antibody, anti-Dig antibody). Capture probes may be purchased with poly-T tails which preferentially adsorb onto native nitrocellulose to provide preferential oriented immobilization (simply requires spotting and drying prior to use). The output DNA may be labeled in different ways: 1) mixing the DNA logic components with the label during the reaction, or 2) carrying out the reaction followed by mixing with the label. Detection may be tested for in both cases using reactions carried out in tubes.

The kinetics of hybridization capture may be tested by running strips and measuring the intensity of gold at the capture line (a flatbed scanner is routinely used to quantify signals). Exposure times for the hybridization reaction may be varied by simply changing the length of the detection strip (flow rate is inversely proportional to the strip length). A kinetic binding curve for the hybridization may be constructed. A time that allows nearly complete capture of the output DNA may be used for subsequent experiments. The high density of surface binding sites can lead to rapid capture, and surface capture sites are normally in large excess compared to analyte, so near 100% capture is typically achievable if given sufficient time. Capture times of less than 5 minutes should be achievable based on immunoassays and the similar binding kinetics for common hybridization reactions. The kinetics of this assay may be further optimized.

DNA Logic Reactions in Paper.

The DNA logic reaction may be carried out as a fluid-phase reaction (similar to the tube-based reaction but with fluid held in paper) or as a solid-phase reaction with immobilized components (an elegant but untested approach). The reaction kinetics for these two approaches may be measured by comparison to the tube-based reactions. Of note, it is expected that immobilization could have significant effects on the reaction kinetics due to constraints on reactant orientation. Real-time measurements of the fluorescence signal may be made directly on the nitrocellulose by using long-wavelength probes to avoid the modest auto fluorescence of nitrocellulose. Alternatively, an end-point measurement at each time point may be made using the visual readout developed above; after a given reaction time, the reaction strip may be manually connected to a detection strip (this approach has been used for development of immunoassays and nucleic acid assays).

Reaction kinetics may be evaluated using the same general framework used in Examples 1 and 2. For the case of immobilized logic circuits, the effective volumetric concentration may be estimated from the known binding capacity for nitrocellulose. Quantitative evaluation of the simplest strand displacement reaction for a single input and single output (FIG. 42) may be performed first, and then may progress to more sophisticated DNA circuit reactions.

The ability to immobilize functional DNA circuits allows for the manipulation of reaction processes that is not possible in homogeneous tube-based reactions; reactions can be spatially-separated (with potential for multiple stage reactions) and exposure of the sample to reaction components can be controlled by flow. DNA circuit components may be biotinylated to allow for immobilization on nitrocellulose with adsorbed streptavidin. It is expected that immobilization and labeling will decrease the rates of strand displacement reactions due to constraints in reactant orientation. A reaction curve may be generated for direct comparison to benchtop reactions via R and k (where R is the reaction rate and k is the reaction rate coefficient with units of concentration/time). Reaction kinetics may be evaluated for different lengths of the poly-T tail for immobilization. A comparison of both R and k should allow for discernment of the fundamental effects due to immobilization (effect on k) versus combined effects of immobilization and high reaction site density (effect on R).

Demonstration of a Paper Strip for Reaction and Visual Detection.

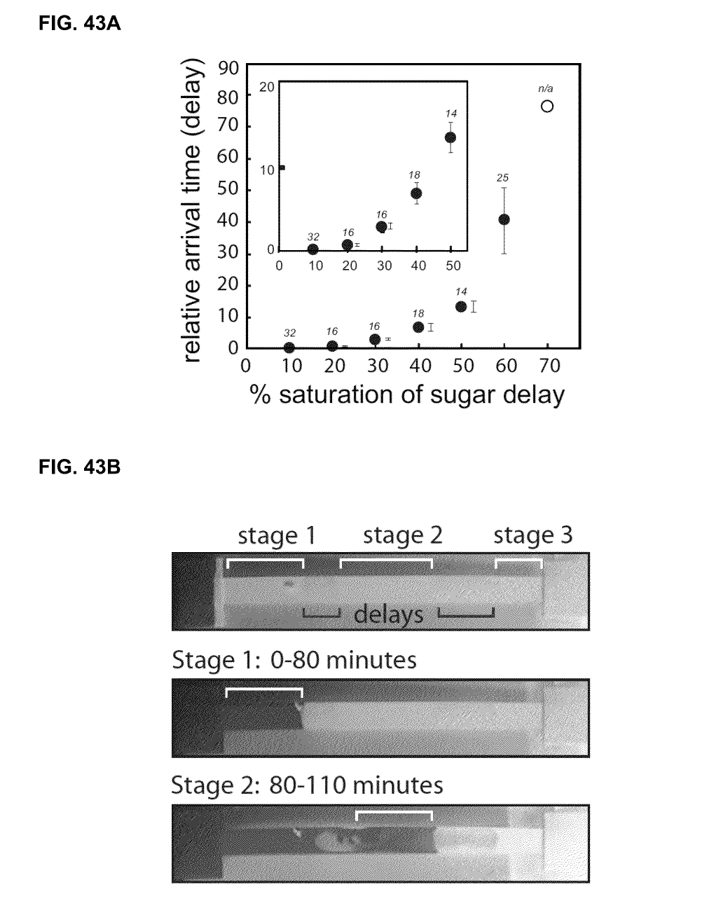

The reagents developed above may be used to demonstrate a simple strip visual readout of DNA circuit reactions like that shown in FIG. 42. The timing for each stage may be set based on the reaction and capture kinetics measured above. Paper-based delay timers using dried sugar solutions were already developed. FIG. 43A shows the delay time (relative to an untreated paper strip); delays can be selected from minutes to over an hour by using solutions of different saturation levels. FIG. 43B demonstrates the use of delay timers to stage fluids in different sections of a simple paper strip, much like the vision image in FIG. 42. Liquid reagents may be used to demonstrate an integrated system. The ability to dry down and rehydrate functional DNA circuit reagents may also be tested; sugar solutions are routinely used as a matrix to store functional reagents in dry form. The simplest DNA logic circuit may be tested in addition to the TB diagnostic analysis from Example 2 using this paper strip based methodology.

In conclusion, DNA-based molecular amplifiers may be designed with single mismatch specificity, to integrate multiple amplifiers into multi-input circuits for diagnostic applications, and to demonstrate circuit operation in a paper-based lateral flow device. This work lays the groundwork for building inexpensive and easy-to-use point-of-care diagnostic devices.

### REFERENCES

The references, patents and published patent applications listed below, and all references cited in the specification above are hereby incorporated by reference in their entirety, as if fully set forth herein.
Abebe, G., F. Paasch, L. Apers, L. Rigouts, and R. Colebunders, Tuberculosis drug resistance testing by molecular methods: Opportunities and challenges in resource limited settings, J. Microbiol. Methods. 84, 155 (2010).
Aldaye, F. A., Palmer, A. L. & Sleiman, H. F. Assembling materials with DNA as the guide. Science 321, 1795-1799 (2008).
Alvarez-Garcia, I. and Miska, E. A., MicroRNA functions in animal development and human disease, Development 132, 4653 (2005).
Arnold, C. et al. Single-nucleotide polymorphism-based differentiation and drug resistance detection in Mycobacterium tuberculosis from isolates or directly from sputum. Clin. Microbiol. Infect. 11, 122-130 (2005).
Ashkenasy, G. & Ghadiri, M. R. Boolean Logic Functions of a Synthetic Peptide Network J. Am. Chem. Soc., 126, 11140-11141 (2004).
Bang, D., A. B. Andersen, and V. O. Thomsen, Rapid genotypic detection of rifampin- and isoniazidresistantn Mycobacterium tuberculosis directly in clinical specimens, J. Clin. Microbiol. 44, 2605 (2006).
Bang, H., Park, S., Hwang, J. & Leel, H. Improved rapid molecular diagnosis of multidrug-resistant tuberculosis using a new reverse hybridization assay, REBA MTB-MDR. Journal of Medical Microbiology 60, 14471454 (2011).
Bartel, D. P. MicroRNAs: target recognition and regulatory functions. Cell 136, 215-233 (2009).
Benenson, Y., Gil, B., Ben-Dor, U., Adar, R. & Shapiro, E. An autonomous molecular computer for logical control of gene expression. Nature 429, 423-429 (2004).
Benenson, Y., Paz-Elizur, T., Adar, R., Keinan, E., Livneh, Z. & Shapiro, E. Programmable and autonomous computing machine made of biomolecules. Nature 414, 430-434 (2001).
Biswas, I., Yamamoto, A. & Hsieh, P. Branch migration through DNA sequence heterology. J. Mol. Biol. 279, 795-806 (1998).
Boehme, C. C. et al. Rapid molecular detection of tuberculosis and rifampin resistance. N. Engl. J. Med. 363, 1005-1015 (2010).
Bois, J. S., Venkataraman, S., Choi, H. M. T., Spakowitz, A. J., Wang, Z. G. & Pierce, N. A. Topological Constraints in Nucleic Acid Hybridization Kinetics. Nucleic Acids Res. 33, 4090-4095 (2005).
Bonnet, G., Tyagi, S., Libchaber, A. & Kramer, F. R. Thermodynamic basis of the enhanced speci_city of structured DNA probes. Proc. Nat. Acad. Sci. 96, 6171-6176 (1999).
Botstein, D., White, R. L., Skolnick, M. & Davis, R. W. Construction of a genetic linkage map in man using restriction fragment length polymorphisms. Am. J. Hum. Genet. 32, 314 (1980).
Brossier, F., N. Veziris, C. Truffot-Pernot, V. Jarlier, and W. Sougakoff, Performance of the genotype MTBDR line probe assay for detection of resistance to rifampin and isoniazid in strains of Mycobacterium tuberculosis with low- and high-level resistance, J. Clin. Microbiol. 44 3659 (2006).
Browning, S. R. & Browning, B. L. Haplotype phasing: existing methods and new developments. Nature Rev. Genetics 12, 703-714 (2011).
Bunka, D. H. J., Platonova, O. & Stockley, P. G. Development of aptamer therapeutics. Curr. Opin. Pharmacol. 10, 557-562 (2010).
Calin, G. A. and Croce, C. M., MicroRNA-cancer connection: the beginning of a new tale, Cancer research 66, 7390 (2006).
Calin, G. A. Human microRNA genes are frequently located at fragile sites and genomic regions involved in cancers Proc. Nat. Acad. Sci. 101, 29993004 (2004).
Cardelli, L. Strand algebras for DNA computing. Lect. Notes. Comput. Sci. 5877, 12-24 (2008).
Carter, D. J. and R. B. Cary, Lateral flow microarrays: a novel platform for rapid nucleic acid detection based on miniaturized lateral flow chromatography, Nucleic Acids Res. 35 (2007).
Cavusoglu, C., A. Turhan, P. Akinci, and I. Soyler, Evaluation of the genotype MTBDR assay for rapid detection of rifampin and isoniazid resistance in Mycobacterium tuberculosis isolates, J. Clin. Microbiol. 44, 2338 (2006).
Chen, X., Ba, Y., Ma, L., Cai, X., et al. Characterization of microRNAs in serum: a novel class of biomarkers for diagnosis of cancer and other diseases, Cell Research 18, 997 (2008).
Corstjens, P., M. Zuiderwijk, A. Brink, S. Li, H. Feindt, R. S, Neidbala, and H. Tanke, Use of upconverting phosphor reporters in lateral-flow assays to detect specific nucleic acid sequences: A rapid, sensitive DNA test to identify human papillomavirus type 16 infection, Clin. Chem. 47, 1885 (2001).
Corstjens, P., M. Zuiderwijk, M. Nilsson, H. Feindt, R. S, Niedbala, and H. J. Tanke, Lateral-flow and up-converting phosphor reporters to detect single-stranded nucleic acids in a sandwich-hybridization assay, Anal. Biochem. 312, 191 (2003).
de Silva, A. P. & Uchiyama, S. Molecular logic and computing Nat. Nanotechnol. 2, 399-410 (2007).
Dirks, R. M., Lin, M., Winfree, E., Pierce, N. A. Paradigms for computational nucleic acid design. Nucl. Acids Res. 32, 1392-1403 (2004).
Dirks, R. M., Bois, J. S., Schae_er, J. M., Winfree, E. & Pierce, N. A. Thermodynamic Analysis of Interacting Nucleic Acid Strands. SIAM Review 49, 65-88 (2007).
Dungchai, W., O. Chailapakul, and C. S. Henry, Use of multiple colorimetric indicators for paper-based microfluidic devices, Analytica Chimica Acta 674 227 (2010).
Edwards, K. A. and A. J. Baeumner, Optimization of DNA-tagged dye-encapsulating liposomes for lateral-flow assays based on sandwich hybridization, Anal. Bioanal. Chem. 386, 1335 (2006).
Egholm, M., Buchardt, O., Nielsen, P. E. & Berg, R. H. Peptide nucleic acids (PNA). Oligonucleotide analogs with an achiral peptide backbone. J. Am. Chem. Soc. 114, 1895-1897 (1992).
Epstein, I. R. & Pojman, J. A. An Introduction to Nonlinear Chemical Dynamics. Oxford University Press, New York, N.Y. (1998).
Esquela-Kerscher, A. and Slack, F. J., Oncomirs: microRNAs with a role in cancer, Nature Reviews Cancer 6, 259 (2006).
Fenton, E., M. Mascarenas, G. Lopez, and S. Sibbett, Multiplex lateral-flow test strips fabricated by two-dimensional shaping, Applied Materials and Interfaces 1 124 (2009).
Frezza, B. M., Cockroft, S. L. & Ghadiri, M. R. Modular multi-level circuits from immobilized DNA-based logic gates. J. Am. Chem. Soc. 129, 14875-14879 (2007).
Friedrich, S. O., A. Venter, X. A. Kayigire, R. Dawson, P. R. Donald, and A. H. Diacon, Suitability of Xpert MTB/RIF and Genotype MTBDRplus for Patient Selection for a Tuberculosis Clinical Trial, J. Clin. Microbiol. 49, 2827 (2011).
Fu, E., B. Lutz, P. Kauffman, and P. Yager, Controlled reagent transport in disposable 2D paper networks, Lab on a Chip 10, 918 (2010a).
Fu, E., P. Kauffman, B. Lutz, and P. Yager, Chemical signal amplification in two-dimensional paper networks, Sensors and Actuators B-Chemical 149 325 (2010b).
Fu, E., S. A. Ramsey, P. Kauffman, B. Lutz, and P. Yager, Transport in two-dimensional paper networks, Microfluidics and Nanofluidics DOI 10.1007/s10404-010-0643-y (2010c).
Gao, Y., Wolf, L. K. & Georgiadis, R. M. Secondary structure effects on DNA hybridization kinetics: a solution versus surface comparison. Nucleic Acids Res. 34, 3370-3377 (2006).
Graugnard, E., Cox, A., Lee, J., Jorcyk, C., Yurke, B., and Hughes, W. Operation of a DNA-based autocatalytic network in serum, Lecture Notes in Computer Science 6518, 83 (2011).
Green, S. J., Lubrich, D. & Turberfield, A. J. DNA hairpins: fuel for autonomous DNA devices. Biophysical Journal 91, 2966-2975 (2006).
Grossmann, T. N. & Seitz, O. Nucleic acid templated reactions: consequences of probe reactivity and readout strategy for amplified signaling and sequence selectivity. Chem. Eur. J. 15, 6723-6730 (2009).
Gunderson, K. L., Steemers, F. J., Lee, G., Mendoza, L. G. & Chee, M. S. A genome-wide scalable SNP genotyping assay using microarray technology. Nature Biotechnology 37, 549-554 (2005).
Guo, Z., Liu, Q. & Smith, L. M. Enhanced discrimination of single nucleotide polymorphisms by artificial mismatch hybridization. Nature Biotechnol. 15, 331-335 (1997).
Hall, J. G. et al. Sensitive detection of DNA polymorphisms by the serial invasive signal amplification reaction. Proc. Natl. Acad. Sci. USA 97, 8272-8277 (2000).
Hillemann, D., M. Weizenegger, T. Kubica, E. Richter, and S, Niemann, Use of the genotype MTBDR assay for rapid detection of rifampin and isoniazid resistance in Mycobacterium tuberculosis complex isolates, J. Clin. Microbiol. 43, 3699 (2005).
Hillemann, D., S. Rusch-Gerdes, and E. Richter, Evaluation of the GenoType MTBDRplus assay for rifampin and isoniazid susceptibility testing of Mycobacterium tuberculosis strains and clinical specimens, J. Clin. Microbiol. 45, 2635 (2007).
Ioannou, Carbon nano-strings as reporters in lateral flow devices for DNA sensing by hybridization, Anal. Bioanal. Chem. 400, 1145 (2011).
Isaacs, F. J., Dwyer, D. J. & Collins, J. J. RNA synthetic biology. Nat. Biotechnol. 24, 545-554 (2006).
Kalogianni, D. P., L. M. Boutsika, P. G. Kouremenou, T. K. Christopoulos, and P. C. Kauffman, P., E. Fu, B. Lutz, and P. Yager, Visualization and measurement of flow in two-dimensional paper networks, Lab on a Chip 10, 2614 (2010).
Kim, J., White, K. S. & Winfree, E. Construction of an in vitro bistable circuit from synthetic transcriptional switches. Molecular Systems Biology 2, 68-79 (2006).
Kim, S. & Misra A. SNP genotyping: technologies and biomedical applications. Annu. Rev. Biomed. Eng. 9, 289-320 (2007).
Koltai, H. & Weingarten-Baror, C. Speci_city of DNA microbarray hybridization: characterization, e_ectors, and approaches for data correction. Nucleic Acids Research 36, 2395-2405 (2008).
Komiyama, M. et al. PNA for one-base differentiating protection of DNA from nuclease and its use for SNPs detection. J. Am. Chem. Soc. 125, 3758-3762 (2003).
Krishnan, Y. and Simmel, F. C., Nucleic Acid Based Molecular Devices 50, 3124 (2011).
Landegren, U., Kaiser, R., Sanders, J. & Hood, L. A ligase-mediated gene detection technique. Science 241, 1077-1080 (1988).
Lawrie, C. H., Gal, S., Dunlop, H. M., Pushkaran, B., et al., Detection of elevated levels of tumour associated microRNAs in serum of patients with diffuse large B-cell lymphoma, British journal of haematology 141 672 (2008).
Lederman, H., Macdonald, J., Stephanovic, D. & Stojanovic, M. N. Deoxyribozyme-based three-input logic gates and construction of a molecular full adder. Biochemistry 45, 1194-1199 (2006).
Li, B., Ellington, A. D. & Chen, X. Rational, modular adaptation of enzyme-free DNA circuits to multiple detection methods. Nucleic Acids Research 39, e110 (2011).
Li, Q., Luan G., Guo, Q. & Liang J. A new class of homogeneous nucleic acid probe based on specific displacement hybridization, Nucleic Acids Res. 30, e5 (2002).
Li, X., J. F. Tian, and W. Shen, Quantitative biomarker assay with microfluidic paper-based analytical devices, Analytical and Bioanalytical Chemistry 396, 495 (2010).
Lishanski, A. Screening for single-nucleotide polymorphisms using branch migration inhibition in PCR-amplified DNA. Clin. Chem. 46, 1464-1470 (2000).
Liu, J. and Cao, Z. and Lu, Y., Functional Nucleic Acid Sensors, Chem. Rev 109, 1948 (2009).
Liu, Y. P., Behr, M. A., Small, P. M. & Kurn, N. Genotypic determination of Mycobacterium tuberculosis antibiotic resistance using a novel mutation detection method, the branch migration inhibition M. tuberculosis antibiotic resistance test. J. Clin. Microbiol. 38, 3656-3662 (2000).
Lu, Y. & Liu, J. Functional DNA nanotechnology: emerging applications of DNAzymes and aptamers. Curr. Opin. Biotechnol. 17, 580-588 (2006).
Lu J. et al. MicroRNA expression profiles classify human cancers. Nature 435, 834-838 (2005).
Makinen, J., H. J. Marttila, M. Marjamaki, M. K. Viljanen, and H. Soini, Comparison of two commercially available DNA line probe assays for detection of multidrug resistant Mycobacterium tuberculosis J. Clin. Microbiol. 44, 350 (2006).
Manganelli, R., Tyagi, S. & Smith, I. Real-time PCR using molecular beacons. Methods Mol. Med. 54, 295-310 (2001).
Mao, X., Y. Q. Ma, A. G. Zhang, L. R. Zhang, L. W. Zeng, and G. D. Liu, Disposable Nucleic Acid Biosensors Based on Gold Nanoparticle Probes and Lateral Flow Strip, Anal. Chem. 81 1660 (2009).
Marras S. A., F. R. Kramer & S. Tyagi. E_ciencies of fluorescence resonance energy transfer and contact-mediated quenching in oligonucleotide probes. Nucleic Acids Research 30, e122 (2002).
Martinez, A. W., S. T. Phillips, E. Carrilho, S. W. Thomas, H. Sindi, and G. M. Whitesides, Simple telemedicine for developing regions: Camera phones and paper-based microfluidic devices for real-time, off-site diagnosis, Analytical Chemistry 80, 3699 (2008).
Martinez, A. W., S. T. Phillips, M. J. Butte, and G. M. Whitesides, Patterned paper as a platform for inexpensive, low-volume, portable bioassays, Angewandte Chemie-International Edition 46 1318 (2007).
McNerney, R. and Daley, P., Towards a point-of-care test for active tuberculosis: obstacles and opportunities, Nature Reviews Microbiology 9, 204 (2011).
Miotto, P., F. Piana, V. Penati, F. Canducci, G. B. Migliori, and D. M. Cirillo, Use of genotype MTBDR assay for molecular detection of rifampin and isoniazid resistance in Mycobacterium tuberculosis clinical strains isolated in Italy, J. Clin. Microbiol. 44 2485 (2006).
Mitchell, P. S., Parkin, R. K., Kroh, E. M., Fritz, B. R. et al., Circulating microRNAs as stable blood-based markers for cancer detection, Proceedings of the National Academy of Sciences 105, 10513 (2008).
Nandagopal, N. & Elowitz M. B. Synthetic biology: integrated gene circuits. Science 333, 1244-1248 (2011).
Navani, N. K. and Li, Y., Nucleic acid aptamers and enzymes as sensors, Current opinion in chemical biology 10, 272 (2006).
Ngom, B., Y. C. Guo, X. L. Wang, and D. R. Bi, Development and application of lateral flow test strip technology for detection of infectious agents and chemical contaminants: a review Anal. Bioanal. Chem. 397 1113 (2010).
Niemz, A., Ferguson, T. M., and Boyle, D. S., Point-of-care nucleic acid testing for infectious diseases, Trends in biotechnology 29, 240 (2011).
Noguera, P., G. A. Posthuma-Trumpie, M. van Tuil, F. J. van der Wal, A. de Boer, A. Moers, and A. Van Amerongen, Carbon nanoparticles in lateral flow methods to detect genes encoding virulence factors of Shiga toxin-producing Escherichia coli, Anal. Bioanal. Chem. 399, 831 (2011).
O'Farrell, B., R. Wong, and H. Tse, Evolution in Lateral Flow-Based Immunoassay Systems Humana Press. p. 1-33. (2009).
Osborn, J., B. Lutz, E. Fu, P. Kauffman, D. Stevens, and P. Yager, Microfluidics without pumps: reinventing the T-sensor and H-filter in paper networks, Lab on a Chip DOI: 10.1039/c004821f (2010).
Panyutin, I. G. & Hsieh P. The kinetics of spontaneous DNA branch migration. Proc. Nat. Acad. Sci. 91, 2021-2025 (1994).
Panyutin, I. G. & Hsieh, P. Formation of a single base mismatch impedes spontaneous DNA branch migration. J. Mol. Biol. 230, 413-424 (1993).
Parsons, L. M. P. L. M., A. Somoskovi, C. Gutierrez, E. Lee, C. N. Paramasivan, A. Abimiku, S. Spector, G. Roscigno, and J. Nkengasong, Laboratory Diagnosis of Tuberculosis in Resource-Poor Countries: Challenges and Opportunities, Clin. Microbiol. Rev. 24 314 (2011).
Peeling, R. W. and D. Mabey, Point-of-care tests for diagnosing infections in the developing world. Clinical Microbiology and Infection 16, 1062 (2010).
Penchovsky R. & Breaker R. R. Computational design and experimental validation of oligonucleotide sensing allosteric ribozymes. Nature Biotech. 23, 1424-1433 (2005).
Peyret, N. Prediction of Nucleic Acid Hybridization: Parameters and Algorithms. Doctoral thesis, Wayne State University. (2000)
Piatek, A. S. et al. Molecular beacon sequence analysis for detecting drug resistance in Mycobacterium tuberculosis. Nature Biotechnol. 16, 359-363 (1998).
Purnick, P. E. M. & Weiss, R. The second wave of synthetic biology: from modules to systems. Nature Rev. Mol. Cell. Biol. 10, 410-422 (2009).
Qian, L. & Winfree, E. Scaling up digital circuit computation with DNA strand displacement cascades. Science 332, 1196-1201 (2011).
Rothemund, P. W. K. Folding DNA to create nanoscale shapes and patterns. Nature 440, 297-302 (2005).
Roush, S, and Slack, F. J., The let-7 family of microRNAs, Trends in cell biology 18, 505 (2008).
Rule, G. S., R. A. Montagna, and R. A. Durst, Rapid method for visual identification of specific DNA sequences based on DNA-tagged liposomes, Clin. Chem., 42 1206 (1996).
Saiki, R. K., Gelfand, D. H., Sto_el, S., Scharf, S. J., Higuchi, R., Horn, G. T., Mullis, K. B. & Erlich, H. A. Primer-directed enzymatic amplification of DNA with a thermostable DNA polymerase. Science 239, 487-491 (1988).
SantaLucia, J. & Hicks, D. The Thermodynamics of DNA Structural Motifs. Ann. Rev. Biochem. 33, 415-440 (2004).
Schena, M., Shalon, D., Davis, R. W. & Brown, P. O. Quantitative Monitoring of Gene Expression Patterns with a Complementary DNA Microarray. Science 270, 467-470 (1995).
Schena, M., Shalon, D., Davis, R. W. & Brown, P. O. Quantitative monitoring of gene expression patterns with a complementary DNA microarray. Science 270, 467-470 (1995).
Seelig, G., Soloveichik, D., Zhang, D. Y. & Winfree, E. Enzyme-free nucleic acid logic circuits. Science 314, 1585-1588 (2006a).
Seelig, G., Yurke, B. & Winfree, E. Catalyzed Relaxation of a Metastable DNA fuel. J. Am. Chem. Soc. 128, 12211-12220 (2006b).
Seeman, N. C., Nanomaterials Based on DNA. Annu. Rev. Biochem. 79, 65-87 (2010).
Severinov, K., Soushko, M., Goldfarb, A. & Nikiforov, V. Rifampicin region revisited. J. Biol. Chem. 268, 14820-14825 (1993).
Shendure, J. et al. Accurate multiplex polony sequencing of an evolved bacterial genome. Science 309, 1728-1732 (2005).
Shih, W. M. & Lin, C. Knitting complex weaves with DNA origami. Curr. Opin. Struct. Biol. 20, 276-282 (2010).
Sidransky, D., Emerging molecular markers of cancer, Nature Reviews Cancer 2, 210 (2002).
Simeonov, A. & Nikiforov, T. T. Single nucleotide polymorphism genotyping using short, fluorescently labeled locked nucleic acid (LNA) probes and fluorescence polarization detection. Nucleic Acids Res. 30, e91 (2002).
Simpson, Z. B., Tsai, T. L., Nguyen, N., Chen, X. & Ellington, A. D. Modelling amorphous computations with transcription networks. J. R. Soc. Interface 6, S523-S533 (2009).
Singh, S. K., Koshkin, A. A., Wengel, J. & Nielsen, P. LNA (locked nucleic acids): synthesis and high-affinity nucleic acid recognition. Chem. Commun. 4, 455-456 (1998).
Soloveichik, D., Seelig, G. & Winfree, E. DNA as a universal substrate for chemical kinetics. Proc. Nat. Acad. Sci. 107, 5393-5398 (2010).
Stojanovic, M. N. & Stefanovic D. A deoxyribozyme-based molecular automaton. Nat. Biotechnol. 21, 1069-1074 (2003).
Stojanovic, M. N., Mitchell, T. E. & Stefanovic D. Deoxyribozyme-based logic gates. J. Am. Chem. Soc. 124, 3555-3561 (2002).
Subramanian, H. K. K., Chakraborty, B., Sha, R. & Seeman, N. C. The label-free unambiguous detection and symbolic display of single nucleotide polymorphisms on DNA origami. Nano Letters 11, 910-913 (2010).
Takahashi, K., Yaegashi, S., Kameda, A. & Hagiya, M. Chain Reaction Systems Based on Loop Dissociation of DNA. Lect. Notes. Comput. Sci. 3892, 347-358 (2006).
Tan, Z. J. & Chen, S. J. Nucleic Acid Helix Stability: Effects of Salt Concentration, Cation Valence and Size, and Chain Length. Biophys. J. 90, 1175-1190 (2006).
Telenti, A. et al. Detection of rifampicin-resistance mutations in Mycobacterium tuberculosis. Lancet 341, 648-650 (1993).
Thompson, B. J., Camien, M. N. & Warner, R. C. Kinetics of branch migration in double-stranded DNA. Proc. Nat. Acad. Sci. 73, 2299-2303 (1976).
Tong, A. K., Li, Z., Jones, G. S., Russo, J. J. & Ju, J. Combinatorial fluorescence energy transfer tags for multiplex biological assays. Nature Biotechnol. 19, 756-759 (2001).
Tsang, J. C. H. and Dennis Lo, Y. M., Circulating nucleic acids in plasma/serum, Pathology 39, 197 (2007).
Tsourkas, A., Behlke, M. A., Rose, S. D. & Bao, G. Hybridization kinetics and thermodynamics of molecular beacons. Nucleic Acids Research 31, 1319-1330 (2003).
Turberfield, A. J. et al. DNA Fuel for Free-running Nanomachines. Phys. Rev. Lett. 90, 118102 (2003).
Tyagi, S. & Kramer, F. R. Molecular beacons: probes that fluoresce upon hybridization. Nature Biotechnology 14, 303-308 (1996).
Tyagi, S. Imaging intracellular RNA distribution and dynamics in living cells. Nature Methods 6, 331-338 (2009).
Urdea, M., L. A. Penny, S. S. Olmsted, M. Y. Giovanni, P. Kaspar, A. Shepherd, P.
Wang, W., W. Y. Wu, W. Wang, and J. J. Zhu, Tree-shaped paper strip for semiquantitative colorimetric detection of protein with self-calibration, Journal of Chromatography A 1217 3896 (2010).
Weinberg, R. A., The biology of cancer, Garland Science New York (2002).
Willner, I., Shlyahovsky, B., Zayats, M. & Willner, B. DNAzymes for sensing, nanobiotechnology, and logic gate applications. Chem. Soc. Rev. 37, 1153-1165 (2008).
Wilson, C. A. Dahl, S. Buchsbaum, G. Moeller, and D. C. Hay Burgess, Requirements for high impact diagnostics in the developing world, Nature 444 Suppl 1, 73 (2006).
Win, M. N. & Smolke, C. D. Higher-order cellular information processing with synthetic RNA devices. Science 322, 456-460 (2008).
Xiao, Y., Plakos, K. J. I., Lou, X., White, R. J., Qian, J., Plaxco, K. W. & Soh, H. T. Fluorescence detection of single-nucleotide polymorphisms with a single, self-complementary, triple-stem DNA probe, Angew. Chemie Int. Ed. 48, 4354-4358 (2009).
Xu, Y., Karalkar, N. B., & Kool, E. T. Nonenzymatic autoligation in direct three color detection of RNA and DNA point mutations. Nature Biotechnol. 19, 148-152 (2001).
Yager, P., G. J. Domingo, and J. Gerdes, Point-of-care diagnostics for global health, Annu Rev Biomed Eng 10 107 (2008).
Yager, P., T. Edwards, E. Fu, K. Helton, K. Nelson, M. Tam, and B. Weigl, Microfluidic diagnostic technologies for global public health, Nature Insight 442 412 (2006)
Yang, Q. et al. Allele-specific Holliday junction formation: a new mechanism of allelic discrimination for SNP scoring. Genome Res. 13, 1754-1764 (2003).
Yashin, R., Rudchenko, S. & Stojanovic, M. N. Networking particles over distance using oligonucleotide based devices. J. Am. Chem. Soc. 129, 15581-15584 (2007).
Yeh, B. J. & Lim, W. A. Synthetic biology: lessons from the history of synthetic organic chemistry. Nat. Chem. Biol. 3, 521-525 (2007).
Yin, P., Choi, H. M. T., Calvert, C. R. & Pierce, N. A. Programming biomolecular self-assembly pathways. Nature 451, 318-322 (2008).
Yurke, B. & Mills, A. P. Using DNA to Power Nanostructures, Genetic Programming and Evolvable Machines 4, 111-122 (2003).
Zhang, D. Y. & Seelig, G., Dynamic DNA Nanotechnology Using Strand Displacement Reactions. Nat. Chem. 3, 103 (2011a).
Zhang, D. Y. and Seelig, G., DNA-based Fixed Gain Amplifiers and Linear Classifier Circuits, DNA 16 Lecture Notes in Computer Science 6518, 176 (2011b).
Zhang, D. Y. & Winfree, E. Control of DNA Strand Displacement Kinetics Using Toehold Exchange. J. Am. Chem. Soc. 131, 17303-17314 (2009).
Zhang, D. Y. & Winfree, E. Robustness and modularity properties of a non-covalent DNA catalytic reaction. Nucleic Acids Res. 38, 4182-4197 (2010).
Zhang, D. Y., Turberfield, A. J., Yurke, B. & Winfree, E. Engineering entropy-driven reactions and networks catalyzed by DNA. Science 318, 1121-1125 (2007).
Zhang, D. Z, Chen. S. X. &Yin, P. Optimizing the specificity of nucleic acid hybridization Nature Chemistry 4, 208-214 (2012).
Zhang, J., Finney, R. P., Clifford, R. J., Derr, L. K. & Buetow, K. H. Genomics 85, 297308 (2005).
Zhao, W. A., M. M. Ali, S. D. Aguirre, M. A. Brook, and Y. F. Li, Paper-Based Bioassays Using Gold Nanoparticle Colorimetric Probes, Analytical Chemistry 80 8431 (2008).

What is claimed is:

1. A detection probe for detecting single base mutations or alterations in a double stranded target nucleic acid molecule comprising:
a double-stranded probe nucleic acid molecule having a first end and a second end;
at least one probe initiation toehold at the first end;
at least one substance capable of emitting a detectable signal at the second end; and
optionally, at least one dissociation toehold at the second end
wherein the detection probe is designed to hybridize with a double stranded target nucleic acid in a reaction which proceeds at approximate thermodynamic equilibrium (ΔG≈0) when no single base mutations or alterations are present in the double stranded target nucleic acid molecule.

2. The detection probe of claim 1, wherein the detection probe hybridizes with the double stranded target nucleic acid in a reaction which proceeds at ΔG<0 when one or more single base mutations or alterations are present in the double stranded target nucleic acid molecule.

3. The detection probe of claim 1, wherein the at least one substance capable of emitting a detectable signal comprises a fluorophore in contact with a quencher.

4. The detection probe of claim 1, wherein the at least one probe initiation toehold, the at least one dissociation toehold, or both, is between approximately 2 nucleotides (nt) and approximately 10 nt long.

5. The detection probe of claim 1, wherein each strand of the double-stranded probe nucleic acid molecule is between approximately 12 nt and approximately 1000 nt long.

6. The detection probe of claim 1, wherein the double-stranded nucleic acid molecule comprises a DNA molecule, an RNA molecule, a peptide nucleic acid (PNA) molecule, or a locked nucleic acid (LNA) molecule.

7. A single base mutation or alteration detection system comprising:
a detection probe which comprises (i) a double stranded probe nucleic acid molecule having a first end and a second end; (ii) at least one probe initiation toehold at the first end; and (iii) at least one substance capable of emitting a detectable signal attached to its second end;
a double stranded target nucleic acid molecule comprising (i) a first end and a second end; and (ii) at least one target initiation toehold at its first end that is complimentary to the probe initiation toehold; and
at least one dissociation toehold at the second end of the detection probe, the second end of the target, or both;
wherein the detection probe is designed to hybridize with the double stranded target nucleic acid molecule in a reaction which proceeds at approximate thermodynamic equilibrium (ΔG≈0) when no single base mutations or alterations are present in the double stranded nucleic acid molecule, and
wherein the detection probe hybridizes with the double stranded target nucleic acid in a reaction which proceeds at ΔG<0 when one or more single base mutations or alterations are present in the double stranded target nucleic acid molecule.

8. The detection system of claim 7, wherein the at least one substance capable of emitting a detectable signal comprises a fluorophore in contact with a quencher.

9. The detection system of claim 7, wherein the at least one probe initiation toehold, the at least one dissociation toehold, or both, is between approximately 2 nucleotides (nt) and approximately 10 nt long.

10. The detection system of claim 7, wherein each strand of the double-stranded probe nucleic acid molecule is between approximately 12 nt and approximately 1000 nt long.

11. The detection system of claim 7, wherein the double-stranded probe nucleic acid molecule comprises a DNA molecule, an RNA molecule, a peptide nucleic acid (PNA) molecule, or a locked nucleic acid (LNA) molecule.

12. The detection system of claim 7, wherein the double stranded target nucleic acid molecule comprises biological DNA or RNA.

13. A method of identifying the presence or absence of one or more single base mutations or alterations in a double-stranded nucleic acid target molecule comprising:
reacting a detection probe with a double-stranded nucleic acid target molecule to produce a reaction product which produces a detectable signal;
measuring a level of the detectable signal of the reaction product;
determining a target hybridization yield via the level of detectable signal;
wherein a target hybridization yield of greater than approximately 10% indicates the absence of one or more single base mutations or alterations in the double-stranded nucleic acid target molecule, and
wherein a target hybridization yield of less than approximately 10% indicates the presence of one or more single base mutations or alterations in the double-stranded nucleic acid target molecule.

14. The method of claim 13, wherein the detection probe comprises (i) a double stranded probe nucleic acid molecule having a first end and a second end; (ii) at least one probe initiation toehold at the first end; (iii) at least one substance capable of emitting a detectable signal attached to its second end; and optionally (iv) at least one dissociation toehold at the second end.

15. The detection system of claim 14, wherein the at least one substance capable of emitting a detectable signal comprises a fluorophore in contact with a quencher.

16. The detection system of claim 14, wherein the double-stranded probe nucleic acid molecule comprises a DNA molecule, an RNA molecule, a peptide nucleic acid (PNA) molecule, or a locked nucleic acid (LNA) molecule.

17. The method of claim 13, wherein the double stranded target nucleic acid molecule comprises (i) a first end and a second end; (ii) at least one target initiation toehold at its first end that is complimentary to the probe initiation toehold; and optionally (iii) at least one dissociation toehold at the second end of the detection probe, the second end of the target, or both.

18. The detection system of claim 13, wherein the double stranded target nucleic acid molecule comprises biological DNA or RNA.

19. The method of claim 13, wherein the detection probe is designed to hybridize with the double stranded target nucleic acid molecule in a reaction which proceeds at approximate thermodynamic equilibrium (ΔG≈0) when no single base mutations or alterations are present in the double stranded nucleic acid molecule.

20. The method of claim 13, wherein the detection probe hybridizes with the double stranded target nucleic acid in a reaction which proceeds at ΔG<0 when one or more single base mutations or alterations are present in the double stranded target nucleic acid molecule.
